(19) United States
(12) Patent Application Publication
(10) Pub. No.: US 2023/0305318 A1
(43) Pub. Date: Sep. 28, 2023

(54) Ophthalmic Devices, Systems and/or Methods for Management of Ocular Conditions and/or Reducing Night Vision Disturbances

(71) Applicant: Brien Holden Vision Institute Limited, Sydney, NSW (AU)

(72) Inventors: Hassan Esfandiarijahromi, Sydney, NSW (AU); Arthur Back, Sydney, NSW (AU)

(21) Appl. No.: 18/021,711

(22) PCT Filed: Aug. 23, 2021
(86) PCT No.: PCT/IB2021/057720

§ 371 Date: Feb. 16, 2023

Related U.S. Application Data
(60) Provisional application No. 63/092,199, filed on Oct. 15, 2020.

(30) Foreign Application Priority Data
Aug. 21, 2020 (WO) PCT/IB2020/057863
Jun. 25, 2021 (WO) PCT/IB2021/055686

(51) Int. Cl. G02C 7/04

(52) U.S. Cl. CPC G02C 7/044; G02C 2202/24

(57) ABSTRACT

An ophthalmic lens configured to correct and/or treat at least one condition of the eye (e.g., presbyopia, myopia, hyperopia, astigmatism, binocular vision disorders and/or visual fatigue syndrome) comprising: a central optical zone; a peripheral optical zone; a base power profile; and at least one feature selected to modify the base power profile and to form one or more off-axis focal points in front of, on, and/or behind a retinal image plane and reduce a focal point energy level at one or more image planes; wherein the at least one feature may be located on a front surface and/or a back surface of at least one of the central optical zone and the peripheral optical zone.

### CROSS REFERENCE TO RELATED APPLICATIONS

This application claims priority to International Application No. PCT/IB2021/055686, filed Jun. 25, 2021; International Application No. PCT/IB2020/057863, filed Aug. 21, 2020; and U.S. Provisional Application No. 63/092,199, filed Oct. 15, 2020. Each of these priority applications are herein incorporated by reference in their entirety.

This application is related to International Application No. PCT/AU2017/051173, filed Oct. 25, 2017, which claims priority to U.S. Provisional Application No. 62/412,507, filed Oct. 25, 2016; International Application No. PCT/IB2020/056079, filed Jun. 26, 2020, which claims priority to U.S. Provisional Application No. 62/868,248, filed Jun. 28, 2019 and U.S. Provisional Application No. 62/896,920, filed Sep. 6, 2019; and U.S. Provisional Application No. 63/044,460, filed Jun. 26, 2020. Each of these related applications are herein incorporated by reference in their entirety.

### TECHNICAL FIELD

This disclosure relates to ophthalmic devices, systems and/or methods for correcting and/or treating refractive errors and/or conditions of the eye. More particularly, this disclosure is related to ophthalmic devices, systems, and/or methods for correcting and/or treating refractive errors and/or conditions of the eye and, in some embodiments, providing low light energy levels for, e.g., further reducing, mitigating or ameliorating night vision dysphotopsias or disturbances. In some embodiments, the ophthalmic lens designs may correct and treat the refractive errors and conditions of the eye by providing an extended depth of focus along the optical axis at least in part on and/or in front of the retina of the eye. In some embodiments, the ophthalmic devices, systems and/or methods may be directed to alleviating night vision disturbances including e.g., any combination of one or more of haloes, glare and/or starbursts and/or for improving vision deficiencies associated with myopia and/or presbyopia.

### BACKGROUND

The discussion of the background in this disclosure is included to explain the context of the disclosed embodiments. This is not to be taken as an admission that the material referred to was published, known, or part of the common general knowledge at the priority date of the embodiments and claims presented in this disclosure.

Ophthalmic devices incorporating simultaneous vision and/or extended depth of field optics may be used for presbyopia correction, for treating refractive errors including myopia control, for alleviating binocular vision disorders and computer vision syndrome. However, there is a need for improved efficacy with use of such devices. Furthermore, although such ophthalmic devices may split light across multiple focal points, they may cause (or at least not alleviate or improve), visual disturbances such as ghosting as well as poor night vision from dysphotopsias or disturbances such as glare, haloes, and starburst to distant light sources.

Accordingly, there is a need to improve the performance of ophthalmic devices e.g., for applications utilizing simultaneous vision and/or extended depth of field optics. The present disclosure is directed to solving these and other problems disclosed herein. The present disclosure is also directed to pointing out one or more advantages to using exemplary ophthalmic devices, systems, and methods described herein.

### SUMMARY

The present disclosure is directed to overcoming and/or ameliorating one or more of the problems described herein.

The present disclosure is directed, at least in part, to ophthalmic devices and/or methods for correcting, slowing, reducing, and/or controlling the progression of myopia.

The present disclosure is directed, at least in part, to ophthalmic devices and/or methods for correcting or substantially correcting presbyopia.

The present disclosure is directed, at least in part, to ophthalmic devices, systems and/or methods to correct and/or treat refractive errors and conditions of the eye including e.g., presbyopia, myopia, astigmatism, binocular vision disorders and/or visual fatigue syndrome and providing low light energy levels for e.g., to further reduce, mitigate or prevent one or more night vision disturbances.

In some embodiments, the method, device, system or feature to correct and/or treat refractive errors and conditions of the eye may incorporate simultaneous optics or extended depth of focus optics to result in a low (e.g., substantially low or moderately low) level of light intensity at the retinal image plane.

In some embodiments, the method, device, system or feature to slow the progression of myopia may incorporate simultaneous optics or extended depth of focus optics to result in a low level of light energy (e.g., low light ray intensity) at the retinal image plane.

In some embodiments, the ophthalmic lens designs may correct and/or treat refractive errors and conditions of the eye by extending the depth of focus along the optical axis at least in part on and/or in front of the retina of the eye during use, and/or further reduce, mitigate or prevent one or more night vision disturbances.

In some embodiments, the ophthalmic lens designs may correct the refractive error(s) of the eye of a user (including e.g., any combination of one or more of a distance refractive error and/or an astigmatic refractive error and/or intermediate and/or a near refractive errors) by extending the depth of focus along the optical axis at least in part on and/or in front of the retina of the eye and/or further reduce, mitigate and/or prevent one or more night vision disturbances.

In some embodiments, the ophthalmic devices, systems and/or methods to manage and/or control refractive errors and conditions of the eye such as presbyopia, myopia, astigmatism, binocular vision disorders and visual fatigue incorporate one or more features to provide low light energy levels and thereby reduce, or mitigate, and/or prevent one or more night vision disturbances including e.g., any combination of one or more of glare, haloes and/or starburst.

In some embodiments, the ophthalmic devices, systems and/or methods incorporating simultaneous and/or extended depth of field optics incorporate an ophthalmic devices, systems and/or methods incorporating simultaneous and/or extended depth of field optics a method, system, or feature to manage one or more night vision disturbances may accompany ophthalmic devices, systems and/or methods incorporating simultaneous and/or extended depth of field optics such that the ophthalmic device, system and/or method results in a low (e.g., substantially low or moderately low) level of light energy along the optical axis of the ophthalmic lens.

In some embodiments, the ophthalmic devices, systems and/or methods incorporating simultaneous and/or extended depth of field optics incorporate a method or system or a feature to manage one or more night vision disturbances such that the ophthalmic device, system, and/or method results in a through focus retinal image quality (RIQ) with one or more independent peaks (e.g., 1, 2, 3, 4, 5, 6, 7, 8, 9, and/or 10 peaks) over a vergence range of about ±3D (e.g., ±2.75 D, ±2.8 D, ±2.9 D±3D, ±3.1 D±3.2 D, and/or ±3.25 D), and wherein the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48).

In some embodiments, the ophthalmic devices, systems and/or methods incorporating simultaneous and/or extended depth of field optics incorporate a method or system or a feature to manage one or more night vision disturbances such that the ophthalmic device, system, and/or method results in through focus retinal image quality (RIQ) with one or more independent peaks (e.g., 1, 2, 3, 4, 5, 6, 7, 8, 9, and/or 10 peaks) over a vergence range of e.g., about ±3D (e.g., ±2.75 D, ±2.8 D, ±2.9 D, ±3D, ±3.1 D, ±3.2 D, and/or ±3.25 D), and/or wherein the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48), and/or wherein the RIQ area (e.g., the area under the through focus RIQ curve bounded by the peak RIQ value and the minimum RIQ value of e.g., 0.11) of the one or more independent peaks may be about 0.16 Units*Diopters (e.g., 0.13, 0.14, 0.15, 0.16, 0.17, 0.18 or 0.19) or less.

In some embodiments, a method or system or a feature to manage one or more night vision disturbances may accompany ophthalmic devices, systems and/or methods incorporating simultaneous and/or extended depth of field optics such that the total enclosed energy that results at the retinal image plane as may be calculated from a light ray distribution such as the retinal spot diagram, may be at least greater than or about 50% (e.g., 45%, 50%, and/or 55%) of the total enclosed energy may be distributed beyond the 35 μm, 40 μm, 45 μm, 50 μm, 55 μm, 60 μm, 65 μm, 70 μm, 75 μm, 80 μm, and/or 95 μm half chord diameter of the retinal spot diagram, and/or may have an average slope of less than about 0.13 units/10 μm (e.g., about 0.11 units/10 μm, 0.12 units/10 μm, 0.125 units/10 μm, 0.13 units/10 μm, 0.14 units/10 μm, and/or 0.15 units/10 μm or less) over 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

The present disclosure is directed, at least in part, to an ophthalmic device, system and/or method to manage one or more night vision disturbances wherein the ophthalmic lens may comprise an optical zone with a base power profile and wherein the optical zone may further comprise a central and a peripheral optical zone.

In some embodiments, the ophthalmic device, system, and/or method to manage one or more night vision disturbances may further comprise a cyclical power profile in the sagittal and/or tangential directions comprising one or more cycles across one or more of the central and/or peripheral optical zones, wherein a cycle of the cyclical power profile in the sagittal and tangential directions incorporates a “m” component that may be relatively more negative in power than the base power of the ophthalmic lens and a “p” component that may be relatively more positive in power than the base power of the ophthalmic lens.

In some embodiments, the ophthalmic device, system, and/or method to manage one or more night vision disturbances may comprise a cyclical power profile comprising one or more cycles across the central and/or peripheral zone of the ophthalmic lens; wherein the peak-to-valley (P-to-V) power range between the absolute powers of the “m” and “p” components of a cycle of the cyclical power profile in a sagittal direction may be about 200 D, about 150 D, about 100 D, about 75 D, about 50 D, about 40 D, about 30 D, about 20 D, about 10 D, about 5 D or less, about 4 D or less, about 3D or less and/or about 2D or less.

In some embodiments, the ophthalmic device, system, and/or method to manage one or more night vision disturbances may comprise a cyclical power profile comprising one or more cycles across the central and/or peripheral zone of the ophthalmic lens; wherein the peak-to-valley (P-to-V) power range between the absolute powers of the “m” and “p” components of a cycle of the cyclical power profile in the tangential direction may be relatively large in order to distribute light energy across a very wide range of vergences (e.g., about 600 D, about 500 D, about 400 D, about 300 D, about 250 D, about 200 D, about 175 D, about 150 D, about 125 D, about 100 D, about 75 D, about 60 D, about 50 D, about 40 D, about 35 D, and/or about 30 D or less).

In some embodiments, the ophthalmic device, system, and/or method to manage one or more night vision disturbances may be a contact lens or an intraocular lens with a central optical zone of half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, and/or about 0.1 mm or less or an absent central optical zone and the ophthalmic lens incorporates a cyclical power profile across the central and/or peripheral zone of the ophthalmic lens; wherein the peak-to-valley (P-to-V) power range between the absolute powers of the “m” and “p” components of a cycle of the cyclical power profile in the sagittal direction may be about 200 D, about 150 D, about 100 D, about 75 D, about 50 D, about 40 D, about 30 D, about 20 D, about 10 D, 5 D, 4 D, 3D, and/or 2D or less, and wherein the peak-to-valley (P-to-V) power range between the absolute powers of the “m” and “p” components of a cycle of the cyclical power profile in the tangential direction may be about 600 D, about 500 D, about 400 D, about 300 D, about 250 D, about 200 D, about 175 D, about 150 D, about 125 D, about 100 D, about 75 D, about 60 D, about 50 D, about 40 D, about 35 D, and/or about 30 D or less, and the frequency of the cyclical power profile in the sagittal direction in at least a portion of the central and/or peripheral optical zone may be about 0.5, 1, 1.5, 2, 5, 10, 20, 50, 100 cycles/mm.

The present disclosure is directed, at least in part, to an ophthalmic lens, system, or method to manage one or more night vision disturbances wherein the ophthalmic lens with a prescribed focal power may comprise a central optical zone of half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, and/or about 0.1 mm or less or an absent central optical zone; the ophthalmic lens may incorporate a cyclical power profile in the sagittal direction in the central and/or peripheral zone with a cycle incorporating a “m” and “p” component and the peak-to-valley (P-to-V) power range between the absolute powers of the “m” and “p” components being about 200 D, about 150 D, about 100 D, about 75 D, about 50 D, about 40 D, about 30 D, about 20 D, about 10 D, about 5 D, about 4 D, about 3D, and/or about 2D or less in the sagittal direction, and a cyclical power profile in the tangential direction in the central and/or peripheral zone with a cycle incorporating a “m” and “p” component and the peak-to-valley power range between the absolute powers of the “m” and “p” components being about 600 D, about 500 D, about 400 D, about 300 D, about 250 D, about 200 D, about 175 D, about 150 D, about 125 D, about 100 D, about 75 D, about 60 D, about 50 D, about 40 D, about 35 D, and/or about 30 D or less in the tangential direction; the frequency of the cyclical power profile in a sagittal direction in at least a portion of the central and/or peripheral optical zone may be about 0.5, 1, 1.5, 2, 5, 10, 20, 50, 100 cycles/mm; and wherein the ophthalmic lens may form one or more off-axis focal points in front of, on, and/or behind the retinal image plane of the eye.

The present disclosure is directed, at least in part, to an ophthalmic lens or system or method to manage one or more night vision disturbances wherein the ophthalmic lens with a prescribed focal power may comprise a central optical zone of half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, and/or about 0.1 mm or less or an absent central optical zone; the ophthalmic lens may incorporate a cyclical power profile in the sagittal direction in the central and/or peripheral zone; with a cycle incorporating a “m” and “p” component and the peak-to-valley power range between the absolute powers of the “m” and “p” components being about 200 D, about 150 D, about 100 D, about 75 D, about 50 D, about 40 D, about 30 D, about 20 D, about 10 D, about 5 D, about 4 D, about 3D, and/or about 2D or less in the sagittal direction, and a cyclical power profile in the tangential direction in the central and/or peripheral zone; with a cycle incorporating a “m” and “p” component and the peak-to-valley power range between the absolute powers of the “m” and “p” components being about 600 D, about 500 D, about 400 D, about 300 D, about 250 D, about 200 D, about 175 D, about 150 D, about 125 D, about 100 D, about 75 D, about 60 D, about 50 D, about 40 D, about 35 D, about 30 D or less in the tangential direction, the frequency of the cyclical power profile in the sagittal direction may be about 0.5, 1, 1.5, 2, 5, 10, 20, 50, 100 cycles/mm and wherein the ophthalmic lens may form one or more off-axis focal points in front of, on, and/or behind the retinal image plane of the eye and wherein at least greater than about 50% of the total enclosed energy may be distributed beyond the 35 μm, 40 μm, 45 μm, 50 μm, 55 μm, 60 μm, 65 μm, 70 μm, 75 μm, 80 μm, and/or 95 μm half chord diameter of the retinal spot diagram, and may have an average slope of less than about 0.13 units/10 μm (e.g., about 0.11 units/10 μm, 0.12 units/10 μm, 0.125 units/10 μm, 0.13 units/10 μm, 0.14 units/10 μm, and/or 0.15 units/10 μm or less) over 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

The present disclosure is directed, at least in part, to an ophthalmic lens or system or method to manage one or more night vision disturbances wherein the ophthalmic lens with a prescribed focal power may comprise a central optical zone of half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, and/or about 0.1 mm or less or an absent central optical zone; the ophthalmic lens may incorporate a cyclical power profile in the sagittal direction in the central and/or peripheral zone; with a cycle incorporating a “m” and “p” component and the peak-to-valley power range between the absolute powers of the “m” and “p” components being about 200 D, about 150 D, about 100 D, about 75 D, about 50 D, about 40 D, about 30 D, about 20 D, about 10 D, about 5 D, about 4 D, about 3D, and/or about 2D or less in the sagittal direction, and a cyclical power profile in the tangential direction in the central and/or peripheral zone; with a cycle incorporating a “m” and “p” component and the peak-to-valley power range between the absolute powers of the “m” and “p” components being about 600 D, about 500 D, about 400 D, about 300 D, about 250 D, about 200 D, about 175 D, about 150 D, about 125 D, about 100 D, about 75 D, about 60 D, about 50 D, about 40 D, about 35 D, and/or about 30 D or less in the tangential direction, the frequency of the cyclical power profile in a sagittal direction may be about 0.5, 1, 1.5, 2, 5, 10, 20, 50, 100 cycle/mm and wherein the through focus retinal image quality (RIQ) has one or more independent peaks over a vergence range of e.g., about ±3.0 D (e.g., ±2.75 D, ±2.8 D, ±2.9 D, ±3D, ±3.1 D, ±3.2 D, and/or ±3.25 D), and the maximum RIQ value of any one of one or more independent peaks may be between about 0.45 (e.g., 0.42, 0.43, 0.44, 0.45, 0.46, 0.47 or 0.48) and about 0.11 (e.g., 0.09, 0.1, 0.11, 0.12, 0.13, 0.14 or 0.15) and wherein the RIQ area (e.g., the area under the through focus RIQ curve bounded by the peak RIQ value and the minimum RIQ value of e.g., 0.11) of the one or more independent peaks may be about 0.16 Units*Diopters (e.g., 0.13, 0.14, 0.15, 0.16, 0.17, 0.18 or 0.19) or less.

The present disclosure is directed, at least in part, to an ophthalmic lens or system or method to manage one or more night vision disturbances wherein the ophthalmic lens with a prescribed focal power may comprise a central optical zone of half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, and/or about 0.1 mm or less or an absent central optical zone; the ophthalmic lens may incorporate a cyclical power profile in the sagittal direction in the central and/or peripheral zone; with a cycle incorporating a “m” and “p” component and the peak-to-valley power range between the absolute powers of the “m” and “p” components being about 200 D, about 150 D, about 100 D, about 75 D, about 50 D, about 40 D, about 30 D, about 20 D, about 10 D, about 5 D, about 4 D, about 3D, and/or about 2D or less in the sagittal direction, and a cyclical power profile in the tangential direction in the central and/or peripheral zone; with a cycle incorporating a “m” and “p” component and the peak-to-valley power range between the absolute powers of the “m” and “p” components being about 600 D, about 500 D, about 400 D, about 300 D, about 250 D, about 200 D, about 175 D, about 150 D, about 125 D, about 100 D, about 75 D, about 60 D, about 50 D, about 40 D, about 35 D, and/or about 30 D or less in the tangential direction, the frequency of the cyclical power profile in the sagittal direction in at least a portion of the central and/or peripheral optical zone being about 0.5, 1, 1.5, 2, 5, 10, 20, 50, 100 cycles/mm and wherein the light from one or more off-axis focal points may be distributed across a substantially wide range of vergences along the optical axis and in front of and/or on and/or behind the retinal image plane of the eye.

The present disclosure is directed, at least in part, to an ophthalmic lens or system or method to manage one or more night vision disturbances wherein the ophthalmic lens with a prescribed focal power may comprise a central optical zone of half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, and/or about 0.1 mm or less or an absent central optical zone; the ophthalmic lens may incorporate a cyclical power profile in the sagittal direction in the central and/or peripheral zone; with a cycle incorporating a “m” and “p” component and the peak-to-valley power range between the absolute powers of the “m” and “p” components being about 200 D, about 150 D, about 100 D, about 75 D, about 50 D, about 40 D, about 30 D, about 20 D, about 10 D, about 5 D, about 4 D, about 3D, and/or about 2D or less in the sagittal direction, and a cyclical power profile in the tangential direction in the central and/or peripheral zone; with a cycle incorporating a “m” and “p” component and the peak-to-valley power range between the absolute powers of the “m” and “p” components being about 600 D, about 500 D, about 400 D, about 300 D, about 250 D, about 200 D, about 175 D, about 150 D, about 125 D, about 100 D, about 75 D, about 60 D, about 50 D, about 40 D, about 35 D, and/or about 30 D or less in the tangential direction, the frequency of the cyclical power profile in the sagittal direction in at least a portion of the central and/or peripheral optical zone being about 0.5, 1, 1.5, 2, 5, 10, 20, 50, 100 cycle/mm and wherein the light energy from one or more narrow optical zones may be distributed across a substantially wide range of vergences along the optical axis of the eye to about +/−100 D or less (sagittal direction) in order to reduce the image quality to within a desired range and more evenly spread the light energy across the retinal image plane and may result in a through focus retinal image quality (RIQ) with one or more independent peaks over a vergence range of e.g., about ±3.0 D (e.g., ±2.75 D, ±2.8 D, ±2.9 D, ±3D, ±3.1 D, ±3.2 D, and/or ±3.25 D), and wherein the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) and wherein the RIQ area of the one or more independent areas may be about 0.16 Units*Diopters (e.g., 0.13, 0.14, 0.15, 0.16, 0.17, 0.18 or 0.19) or less.

In some embodiments, the light passing through the off-axis focal points formed by the at least one or more narrow optical zones may intersect the optical axis and may form at least one or more (including e.g., an infinite number) on-axis focal points along the optical axis that may be distributed across a very wide range of vergences along the optical axis of the eye, in front of, on, and/or behind the retinal image plane, and may have low light energy level of the images of objects formed on the retina, and/or may have a uniform or relatively uniform light ray intensity distribution across the retinal spot diagram wherein at least greater than about 50% of the total enclosed energy may be distributed beyond the 35 μm, 40 μm, 45 μm, 50 μm, 55 μm, 60 μm, 65 μm, 70 μm, 75 μm, 80 μm, and/or 95 μm half chord diameter of the retinal spot diagram and may have an average slope of less than about 0.13 units/10 μm (e.g., about 0.11 units/10 μm, 0.12 units/10 μm, 0.125 units/10 μm, 0.13 units/10 μm, 0.14 units/10 μm, and/or 0.15 units/10 μm or less) over 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10·m, and/or 0.15 units/10 μm).

In some embodiments, the ophthalmic lenses may include optical designs comprising at least one or more narrow optical zones incorporating cyclical power profiles in both sagittal and tangential directions and forming at least one or more off-axis focal points and at least one or more (including e.g., an infinite number) on-axis focal points along the optical axis that may have low light energy and may provide, at least in part, an extended depth of focus within a useable vergence ranges encountered by the user of the ophthalmic lens.

Other features and advantages of the subject matter described herein will be apparent from the description and drawings, and from the claims

### BRIEF DESCRIPTION OF THE DRAWINGS

Aspects of the embodiments described herein may be understood from the following detailed description when read with the accompanying figures.

FIG. 1 illustrates plan and cross-sectional views of an ophthalmic lens incorporating an exemplary optical design in accordance with some embodiments described herein, wherein the plurality of narrow optical zones in the peripheral zone may be formed by a line curvature.

FIG. 2A, FIG. 2B, and FIG. 2C are schematic diagrams of light rays from a far distance object traced through an exemplary ophthalmic lens of FIG. 1 incorporating an exemplary optical design in accordance with some embodiments described herein, wherein the plurality of narrow optical zones in the peripheral zone may be formed by a line curvature. FIGS. 2A and 2B provide detailed views of the on and off-axis focal points formed by the light rays after passing through the ophthalmic lens and anterior eye optical system and FIG. 2C illustrate a light ray distribution at the retinal image plane.

FIG. 3A and FIG. 3B illustrate Zemax simulations of the cyclical power profile (sagittal and tangential) produced by the exemplary ophthalmic lens described in FIG. 1 incorporating an exemplary optical design in accordance with some embodiments described herein.

FIG. 4 illustrates the retinal image quality (RIQ i.e., Visual Strehl Ratio) for a 5 mm pupil and for a wavelength of light of 589 nm along the optical axis of an ophthalmic lens of FIG. 1 incorporating an exemplary optical design in accordance with some embodiments described herein.

FIG. 5A and FIG. 5B illustrate a Zemax optical simulation of the light energy distribution (spatial distribution (FIG. 5A) and fractional distribution (FIG. 5B)) across the retinal spot diagram at the retinal image plane of an ophthalmic lens o from FIG. 1 incorporating an exemplary optical design in accordance with some embodiments described herein.

FIGS. 6A-U illustrate a tabulated summary of exemplary lens designs (FIG. 6A), optical parameters and simulated optical modeling metrics (FIGS. 6B-6U) for the ophthalmic lenses in FIG. 6A incorporating exemplary optical designs in accordance with some embodiments described herein.

FIGS. 7A-F plot several more exemplary patterns of cyclical on-axis power profiles (sagittal) for ophthalmic lenses that may be configured by incorporating exemplary optical designs in accordance with some embodiments described herein.

FIG. 8 is a schematic diagram of select light rays from a far distance object traced through an exemplary ophthalmic lens and anterior eye optical system incorporating an exemplary optical design in accordance with some embodiments described herein, and illustrating an embodiment having optical zones configured to form off-axis focal points in front of the retinal plane e.g., a real image inside the eye and behind (e.g., more posteriorly than) the cornea.

FIG. 9 is a schematic diagram of select light rays from a far distance object traced through an exemplary ophthalmic lens and anterior eye optical system incorporating an exemplary optical design in accordance with some embodiments described herein, and illustrating an embodiment having optical zones configured that do not form off-axis focal points in front of or behind the retinal plane (e.g., no image inside, in front of or behind the eye).

FIG. 10 is a schematic diagram of select light rays from a far distance object traced through an exemplary ophthalmic lens and anterior eye optical system incorporating an exemplary optical design in accordance with some embodiments described herein, and illustrating an embodiment having optical zones configured to form off-axis focal points in front of the cornea (e.g., virtually outside of the eye more anteriorly in front of the cornea).

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The subject headings used in the detailed description are included for the ease of reference of the reader and should not be used to limit the subject matter found throughout the disclosure or the claims. The subject headings should not be used in construing the scope of the claims or the claim limitations.

The terms “about” as used in this disclosure is to be understood to be interchangeable with the term approximate or approximately.

The term “comprise” and its derivatives (e.g., comprises, comprising) as used in this disclosure is to be taken to be inclusive of features to which it refers, and is not meant to exclude the presence of additional features unless otherwise stated or implied.

The term “myopia” or “myopic” as used in this disclosure is intended to refer to an eye that is already myopic, is pre myopic, or has a refractive condition that is progressing towards myopia.

The term “presbyopia” or “presbyopic” as used in this disclosure is intended to refer to an eye that is has a diminished ability to focus on intermediate and near objects.

The term “ophthalmic lens” or “ophthalmic device” as used in this disclosure is intended to include one or more of a contact lens, or an intraocular lens, or a spectacle lens.

The term “night vision disturbances” or “night vision dysphotopsias” refer to any combination of one or more symptoms of haloes, glare and star bursts for distant objects. Methods for assessing the existence and/or reduction of night vision disturbances are well known in that art. For example, one subjective assessment of “lack of night vision disturbances” may involve measurement of “starbursts” ranked on an analog scale of 1-10 where 1=absent and 10=excessive, or on a Likert scale of good (no starburst), average (some starburst) and poor (excessive). In some embodiments, a reduction in subjective assessment of 1 unit or more may be considered to be reduction and/or minimization of night vision disturbance.

The term “low light energy levels” or “low light level” of an ophthalmic lens as used in this disclosure is intended to refer to a reduction in the amount of light at a given vergence and may be measured by the retinal image quality (RIQ) at that given vergence. Values of RIQ that may qualify as low light energy levels or low light levels may be approximately 50% or less (e.g., 0.5 or less), or about 45% or less (e.g., 0.45 or less) as compared to the RIQ of the diffraction limited lens at that given vergence and the area under the maximum peak RIQ value may be less than about 0.16 unit*Diopter where the range of vergences may be +/−3.00 D. A peak RIQ area may be defined as the area enclosed by the through focus RIQ curve beneath an independent peak (maximum peak RIQ value of between about 0.11 to about 0.45) and wherein the RIQ curve falls below about 0.11 on at least the side of the RIQ peak with the lower vergence value.

The term “focal point energy level” or “focal point energy” as used in this disclosure refers to the RIQ value at the vergence of that focal point at the image plane.

The term “line curvature” as used in this disclosure refers to a geometrically three-dimensional surface, wherein along at least one direction of that surface, a “portion” of a two-dimensional line or of a “substantially” two-dimensional line may be observed. For example, a line curvature may be created by the revolution of a “portion” of a two-dimensional line or of a “substantially” two-dimensional line on an annular zone around the central axis of an ophthalmic lens, and wherein a revolution curvature may be observed along a secondary direction for example, circumferentially.

The term “model eye” as used in this disclosure is used to determine the through focus RIQ curve, retinal spot diagram and the enclosed energy diagram and refers to a Navarro-Escudero eye modified to mimic presbyopic eyes with no accommodation and the ray-tracing routines performed in a ray tracing program (e.g., ZEMAX, FOCUS software) with the aberration terms optimized to zero.

There is a need for ophthalmic lens designs incorporating multifocal and extended depth of focus optics to improve efficacy with vision correction and/or vision treatment. A limitation of ophthalmic lens designs incorporating multifocal and extended depth of focus optics for vision correction and/or vision treatment based on the simultaneous vision optics has been the interference of out-of-focus images with the in-focus images; this may result in visual disturbances such as ghosting and/or night vision disturbances including, e.g., any combination of glare, haloes, and starbursts. For example, with ophthalmic lenses designed to provide extended depth of focus for presbyopia management, attention may be primarily targeted to providing the highest RIQ over an extended range of vergences rather than management of visual compromises, including night vision disturbances. Likewise, in vision treatments directed to slowing myopia, attention is primarily targeted to providing a higher RIQ on and/or in front of the retina than behind the retina. Typically, night vision disturbances may arise when ophthalmic lens designs incorporating multifocal and/or extended depth of focus optics provide a light distribution across the retinal image plane that may not be optimized, for example, because the intensity of defocused on-axis light rays from other image planes arriving at the retinal plane may be too high and/or concentrated and/or intense and may interfere and/or compete with the in focus light rays at the retinal plane. In addition to interfering with efficacy, they may produce visual compromises such as for example, ghosting by interfering with the in focus light energy. Also, the excessively high and/or concentrated and/or intense defocused light energy at the retinal plane may result in night vision disturbances such as glare, haloes, and/or starbursts. Consequently, some embodiments may relate to ophthalmic lens designs incorporating multifocal and extended depth of focus optics for vision correction and/or vision treatment by controlling the image quality of on-axis focal points across the through focus vergences to reduce the interference of out-of-focus images on in-focus images at the retinal image plane, and to provide a relatively even distribution of the light energy intensity with less interference from out-of-focus light rays at the retinal image plane and thereby reducing and/or mitigating night vision disturbances such as glare, haloes and starbursts. Therefore, some embodiments disclosed herein may provide ophthalmic lens designs incorporating extended depth of focus technology for vision correction and/or vision treatment and to provide desirable/optimal levels of image qualities along the optical axis and desirable/optimal light energy distribution across the retinal image plane to provide low light energy levels and reduce, mitigate and or prevent or night vision disturbances such as glare, haloes and/or starbursts.

In some embodiments, the ophthalmic lens may include an optical design formed on a lens surface, for example a front surface and/or a back surface, that may be configured with an optical zone with a base power, the optical zone comprising a small central zone that may form, for example, a focal point along the optical axis, in front of, and/or on, and/or behind the retinal image plane and may be surrounded by an annular peripheral zone comprising at least one or more narrow and/or annular conjoined optical zones that may have a cyclical power profile in a sagittal and a tangential directions that may be configured to form at least one or more off-axis focal points, for example in front of the retinal image plane, and may also result in at least one or more on-axis focal points when light rays from the off-axis focal points intersect along the optical axis, for example, in front of, and/or on, and/or behind the retinal image plane, and/or in front of, and/or, on, and/or behind the on-axis focal point formed by the central optical zone. In some embodiments, narrow and/or annular optical zones located in the central and/or peripheral zone may also be configured to provide a light energy distribution along the optical axis and may be distributed over a wide range of vergences and be of a defined low intensity. In some embodiments, the low intensity light energy distributed along the optical axis may form a light intensity across the retinal image plane that may also be uniform, for example evenly distributed over the retinal spot diagram. In some embodiments, the central zone may also be configured to provide at least one or more focal point(s) along the optical axis that may also be of low intensity, for example by sizing the central zone at a dimension small enough to reduce the light intensity of the focal point within defined value ranges. In some embodiments, the light intensity and distribution along the optical axis formed by the central zone may also form a light intensity on the retina that may also be of low intensity and/or may be uniform, for example evenly distributed over the retinal spot diagram.

In some embodiments, the light energy distribution along the optical axis, for example on-axis focal points, formed by the central zone and/or the narrow and/or annular optical zones of the peripheral zone may combine to provide an extended depth of focus, that may be formed over a range of vergences useful for vision correction including correcting myopia, hyperopia, presbyopia, astigmatism and/or any combinations thereof or for binocular vision orders and visual fatigue syndromes. In some embodiments, the on-axis focal points formed by the central zone and/or the narrow and/or annular optical zones of the peripheral zone may combine to provide an extended depth of focus, that may be formed over a range of vergences along the optical axis useful for controlling the progression of myopia. In some embodiments, the distribution and/or the intensity of the on-axis focal points formed by the central zone and/or the narrow and/or annular optical zones of the peripheral zone may combine to provide a light intensity on the retina that may be of low intensity and/or of relatively uniform intensity over the retinal spot diagram that may slow, reduce or control the progression of myopia. In some embodiments, the distribution and/or the intensity of the on-axis focal points formed by the central zone and/or the narrow and/or annular optical zones of the peripheral zone may combine to provide a light energy on the retina that may be of low energy and/or of relatively uniform intensity over the retinal spot diagram that may reduce, mitigate or prevent night vision dysphotopsias such as glare, haloes, and/or starbursts.

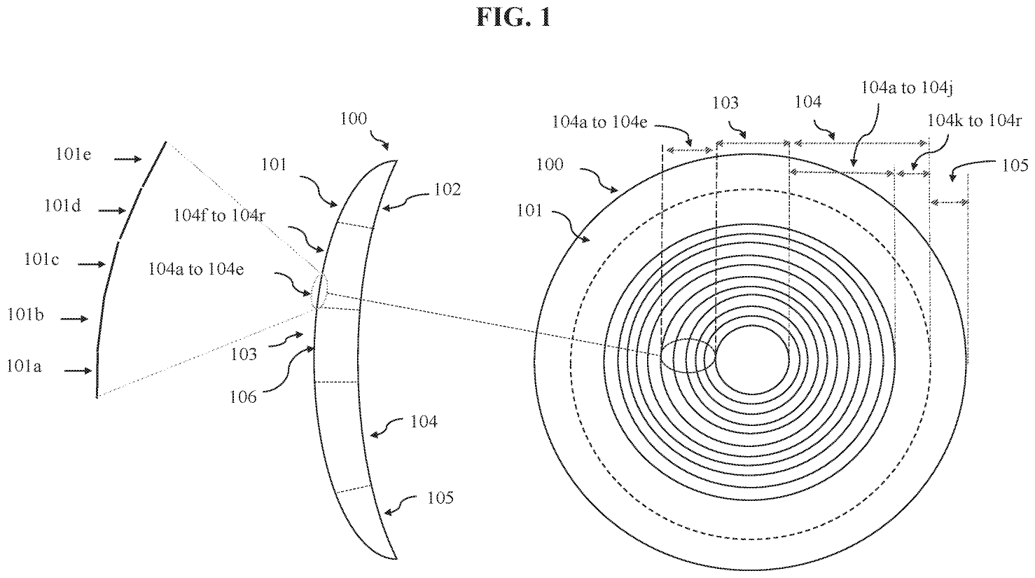

FIG. 1 illustrates a cross-sectional and a plan view of an exemplary embodiment of an ophthalmic lens, for example a contact lens, that may provide an extended depth of focus useful for vision correction and/or vision treatment and that may also reduce, or mitigate, or prevent one or more night vision disturbances.

The ophthalmic lens with a base power profile 100 comprises a front surface 101, a back surface 102, a central zone 103 and peripheral zones 104 and 105. The central zone 103 may have a diameter of about 1.0 mm and may be formed by a surface curvature 106 to form a power profile that when combined with the back surface curvature 102, the lens thickness and refractive index may produce at least one focal point along the optical axis in front of the retina 208. The peripheral zone 104 incorporates a plurality of narrow annular concentric optical zones 104a to 104r that are about 200 μm wide, are located on the front surface 101 and may be formed by corresponding line curvatures 101a-101r and the resulting surface of the peripheral optical zone may be configured as a smooth and/or continuous surface e.g., without surface discontinuities. In some embodiments, the surface of the peripheral optical zone incorporating the plurality of narrow optical zones may not be configured as smooth and/or continuous (e.g. they may include one or more surface discontinuities). To simplify the diagram, only the first 10 narrow optical zones 104a to 104j are shown in the plan view and the remaining narrow optical zones 104k to 104r are not drawn (appearing as a blank space 107) in the outer portion of the peripheral zone 104 while the cross-sectional view includes only the first 5 line curvatures 101a to 101e that may configure the first 5 narrow optical zones 104a to 104e on the front surface of the peripheral zone 104. The net resultant power profile of the narrow annular zones 104a-104r of the peripheral zone 104 may be relatively more positive in power than the central zone 103. The plurality of narrow annular concentric optical zones 104a to 104r may be conjoined with an adjacent narrow annular concentric optical zone and may be formed by at least one line curvature. Additionally, the narrow annular concentric zones may be configured so that the innermost and outermost portions of the at least one narrow optical zones may be geometrically normal to the surface and may provide a lateral separation of the focal points (e.g., the infinite number of focal points) formed by the annular narrow optical zones from the optical axis 207. A conjoined zone may exist when the spacing between the two adjacent optical zones may be about 0 mm and the innermost and the outermost portion of the surface curvature of the narrow optical zones may transition to the base curve (e.g., the curvature of the first or the base optical zone) or base curve of the peripheral zone. In some embodiments, at least one of the plurality of narrow zones may be conjoined with a second narrow zone (e.g. 104a and 104b). In some other embodiments, the at least one of the plurality of narrow optical zones may be spaced apart and, for example, the power profiles may alternate wherein at least one or more of the plurality of narrow zones may have a first power profile and at least one or more of a plurality of narrow zones may have a different power profile.

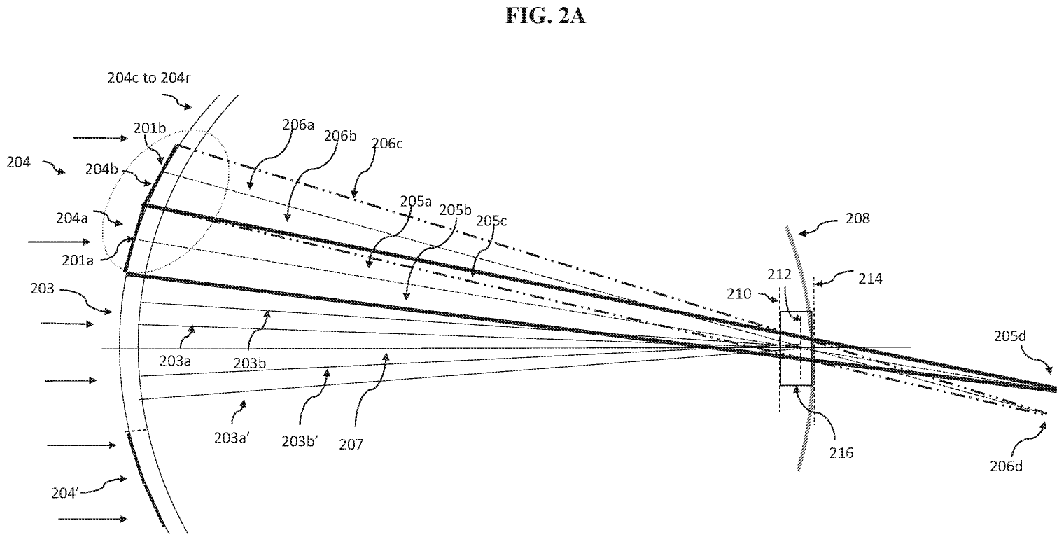

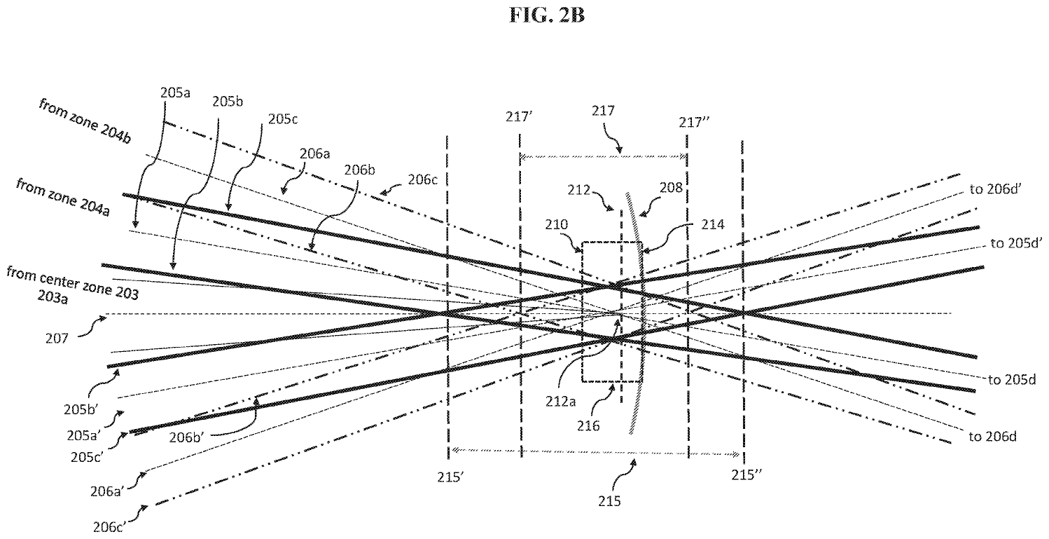

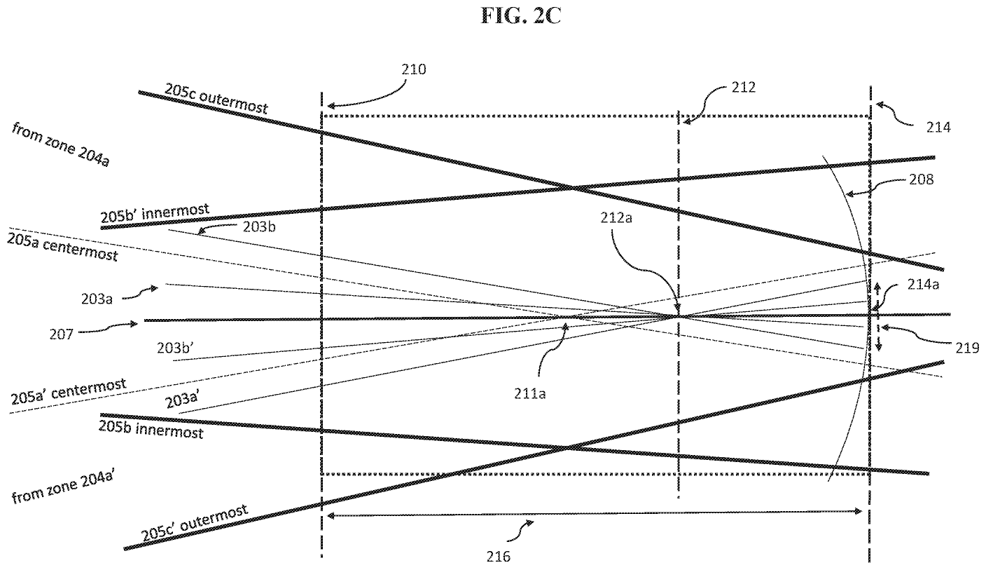

FIGS. 2A, 2B and 2C illustrate different views of a schematic ray diagram for parallel light rays originating from a distant object and passing through the example ophthalmic lens of FIG. 1 and the optics of a simplified eye model and forming on-axis and off-axis focal points at multiple image planes. The schematic ray diagram illustrated in FIG. 2A provides an overview of the light rays propagating through the optical system as described. For purposes of clarity, representative light rays are only shown for a portion of the center zone 203 and for the upper portion of the lens and for only 2 (204a, 204b) of the 18 narrow annular conjoined optical zones (previously referred to as 104a and 104b in FIG. 1) of the peripheral zone 204. The view of the schematic ray diagram illustrated in FIG. 2B provides zoomed in details of the distribution of representative light rays in front of the eye, within the eye and behind the retinal image plane 208 by the center zone and the centermost, innermost and outermost portions of the narrow optical zones 204a and 204b. The zoomed in view of the schematic ray diagram illustrated in FIG. 2C provides further zoomed in details of focused and defocused representative light rays formed by the center zone 203 and the first narrow annular optical zone 204a along the optical axis across a depth of focus 216 over a vergence in front of the retina 210 to the retinal image plane 214.

In some embodiments, the power profile of the central zone 203 may be relatively more positive than the power required to correct the distance refractive error of the eye of the user and accordingly, as illustrated in FIGS. 2A and 2B, the light rays 203a, 203b from the central zone 203 converge to form a focal point 212a along the optical axis at image plane 212 in front of the retinal image plane 214. Importantly, the focal point 212a formed by the center zone 203 may be a reduced energy focal point. Light rays subsequently diverge from the focal point 212a and may reach the retinal image plane 214 forming a defocused image on the retinal image plane 214 over distance 219 (FIG. 2C).

As seen in FIG. 2A, 2 of the plurality of the narrow annular conjoined optical zones 204a to 204b in the peripheral zone 204 may be configured with a surface geometry and a power profile to laterally separate the focal points from the optical axis and form off-axis focal points 205d and 206d behind the retinal image plane 214. The front surface line curvatures 201a and 201b forming the narrow optical zones may be configured geometrically as normal to the surface and in some embodiments, the optical axes e.g., the centermost rays 205a and 206a (and 205a′ and 206a′ from the bottom portion on the ray diagram cross-section in FIG. 2B) of the narrow optical zones 204a-204b (FIG. 2B) may intersect the optical axis 207 and form on-axis focal point 211a at image plane in front of the reduced light energy coaxial focal point 212a from the center zone 203 (see, e.g., FIG. 2C). FIG. 2B shows the light rays from the innermost (205b, 206b) and outermost (205c, 206c) portions of the narrow optical zones 204a and 204b may intersect the optical axis 207 across a wide range of vergences, for example the zone 204a disperses the light energy over distance 215 (e.g., 15 D) between 215′ and 215″ and the second optical zone 204b disperses the light energy over distance 217 (e.g., 11 D) between 217′ and 217″, Dispersing the light energy over distance 215 and 217 may be substantially beyond an extended depth of focus 216 (e.g., about 2D to 3D) between image planes 210 and 214 required for useful vision correction and/or vision treatment and accordingly the light energy contributing to forming focal points along the optical axis over distance 217 and also the depth of focus 216 may be reduced to lower levels. Likewise, the retinal image quality (RIQ) along the optical axis may also be low but importantly may have sufficient image quality to provide an extended depth of focus useful for vision correction and/or vision treatment by reducing/minimizing interference of low energy in focus images by also lowering the energy level of out of focus images and overcoming one or more limitations of simultaneous vision lenses. FIG. 2C provides a zoomed in view of the ray diagram from a representative sample of light rays from the center zone 203 and the first narrow optical zone 204a of the peripheral zone 204 (FIG. 2A) over the distance 216 between focal plane 210 and the retinal image plane 214 and may correspond to about the depth of focus provided by the example lens from FIG. 1 (e.g., about 2D). The light rays from the small center zone 203 form a reduced energy focal point at 212a and subsequently form a defocused image, also of reduced energy, on the retinal image plane 214 over about distance 219. In addition, further low energy defocused images may be formed over the retinal image plane by defocused light rays from the narrow optical zones such as the centermost light rays (205a) from a reduced energy focal point 211a and light rays from a portion of the zone 204a between the innermost (205b) and outermost (205c) light rays converging to focal point 205d or diverging after intersecting the optical axis and these rays may be of sufficiently low intensity and sufficiently evenly distributed across the retinal image plane that the in focus retinal image used for far vision at night may have reduced night visual disturbances from e.g., glare, haloes and/or starbursts.

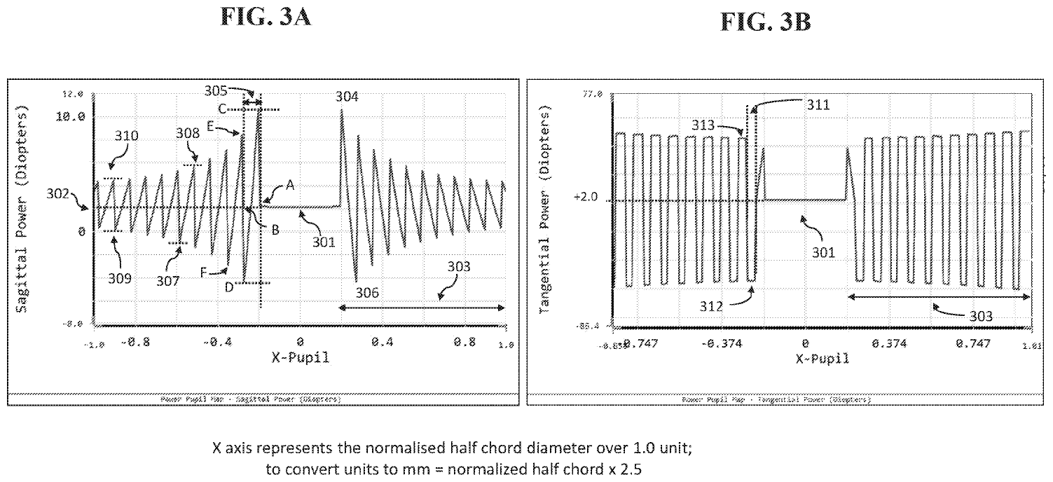

FIGS. 3A and 3B are schematic plots of the on-axis power profile of the central zone 103 and a portion of the peripheral zone 104 of the ophthalmic lens described in FIG. 1, modeled in optical design software (Zemax) in both the sagittal (FIG. 3A) and tangential (FIG. 3B) directions. The horizontal axis of the power plot is the normalized half chord diameter over a unit of +/−1 from the lens center and so 1 unit represents a 2.5 mm half chord diameter on the ophthalmic lens. The central zone 103 of the ophthalmic lens 100 forms a constant power profile 301 of about +2.25 D over the 1.0 mm diameter. In some embodiments, the central zone power 301 of the ophthalmic lens may be more positively powered than the refractive error of the eye (e.g., nominally set at +2.25 D for a +1.75 D spherical refractive error) and therefore may form a coaxial focal point 212a in front of the retina, as detailed in FIG. 2B. In some embodiments, the central zone power profile 301 may be configured to correct the far refractive error and in some embodiments the center zone power profile may be configured to focus at a vergence other than the far refractive error of the eye. The power profile of a portion, for example about 2 mm width (303) of the peripheral optical zone 104 comprising a plurality of narrow optical zones (e.g., 10 zones) 104a to 104j illustrated in FIG. 1 shows cyclical power profiles in both sagittal and tangential directions. In the sagittal direction, the narrow optical zones of the peripheral zone forms a single cycle of oscillation of power, for example at 305 between A and B, around the base power of the center zone power 301. In some embodiments, the cyclical power profile of the narrow optical zone may oscillate around the base lens power of the peripheral zone. The power profile cycles, for example in the sagittal direction, may form a more positive (“p” e.g., 304) and a more negative (“m” e.g., 306) component relative to central zone power 301 that may arise from the geometrical normal to the surface configuration of the narrow optical zones. In some embodiments, a line curvature may be used to form the narrow optical zones wherein the power changes within a cycle in the sagittal direction may be linear between the p and m components and passing through the center zone power. In some embodiments, at least two or more-line curvatures may be used to form a narrow optical zone and therefore may be used to provide a different linear power profiles or any shape of power progression by using a greater number of line curvatures within a zone. In some embodiments, at least one line curvature may be used in conjunction with any other surface curvature e.g., at least one spherical or aspherical curvature to provide a curvilinear power profile or any shape of power progression. In some embodiments, any curvature may be used to provide a power profile with any shape and/or slope of progression within a cycle. The absolute power range between the “p” and “m” components in the single power profile cycle e.g., in the sagittal direction between C and D in the first cycle 305 (the peak to valley or P-V value) of the first and second (between E and F) narrow optical zones of the peripheral region 104 of example lens 100 from FIG. 1 is about 15 D and about 11 D respectively and the P-V value decreases in value across the peripheral region e.g., between 307-308 and 309-310. In some embodiments, the P-V values may be constant or may not be constant. In some embodiments, the P-V values may increase or decrease or remain constant for at least 2 of the cycles or may be randomly changing. The high-powered cyclical power profiles in the optical zones, for example in the sagittal direction (FIG. 3A), may disperse the light energy across a wide range of vergences along the optical axis, for example over distance 215 and 217 for the first and second narrow optical zones 204a and 204b as illustrated in FIG. 2B and thereby reducing the light energy of focal points formed along the optical axis. In some embodiments, the first cycle of the cyclical power profile in, for example the sagittal direction, originating from the first narrow optical zone of the peripheral zone adjacent to the center zone e.g. at 305 may begin with the power profile in the narrow optical zone increasing from A in relatively more positive power than the base center zone power to a maximum more positive power e.g., the ‘p’ or most positive powered component of the cycle and then the power profile may decrease in relatively more negative power than the ‘p’ component and the base center zone power to reach a maximum more negative power e.g., the ‘m’ or most negative powered component. A single cyclical power profile in the sagittal direction may be completed when the power returns to the base power of the center zone e.g., at B. In some embodiments, the first cycle may first reach or pass through the p component or may first reach the m component.

FIG. 3B shows the tangential power map for the example ophthalmic lens described in FIGS. 1 and 2. The cycles of the cyclical power profiles formed by the narrow optical zones e.g., 305 (FIG. 3A) configured with conjoined line curvatures on the front surface shaped geometrically normal to the surface (plano-concave lens cross section) may form high minus off-axis power values e.g., of −55 D at 312 inside the single optical zone (e.g. the power at 311 is formed over a smaller dimension than a single cycle 305). The boundaries between the conjoined annular zones on the object side of the lens front surface may form surface contours e.g. a surface contour formed by an outer portion of the first narrow optical zone 104a and an inner portion of the second narrow optical zone 104b (FIG. 1A) at about their boundary, and create a boundary power that may also form high positive off-axis power values e.g., +46 D at 313 by the narrow optical zones 104a and 104b. In some embodiments, the high cyclical power values in the sagittal (FIG. 3A) and tangential (FIG. 3B) direction may contribute to the dispersion of light energy over a very wide range of vergences along the optical axis as illustrated and described in FIG. 2B.

The through focus image quality along the optical axis of the ophthalmic lens may be measured by one or more metrics such as the visual strehl ratio and may be determined as the ratio of the integration of the MTF values across the desired spatial frequencies e.g., 0-30 cycles/degree of the image at the vergences along the optical axis divided by the integration of the MTF values across the desired spatial frequencies e.g. 0-30 cycles/degree of an image formed by the equal diffraction limited lens and ranked as 1-0 wherein 1=perfect image quality and 0=poor image quality. The image quality metric may encompass both the intensity of light rays focused at the image plane as well as the intensity of any defocused light rays converging or diverging toward the image plane, and thus the image quality is a sum of higher intensity light rays formed by on-axis optical zones at the image plane as well as interference from any light energy emanating from any other on-axis and off-axis optical zones.

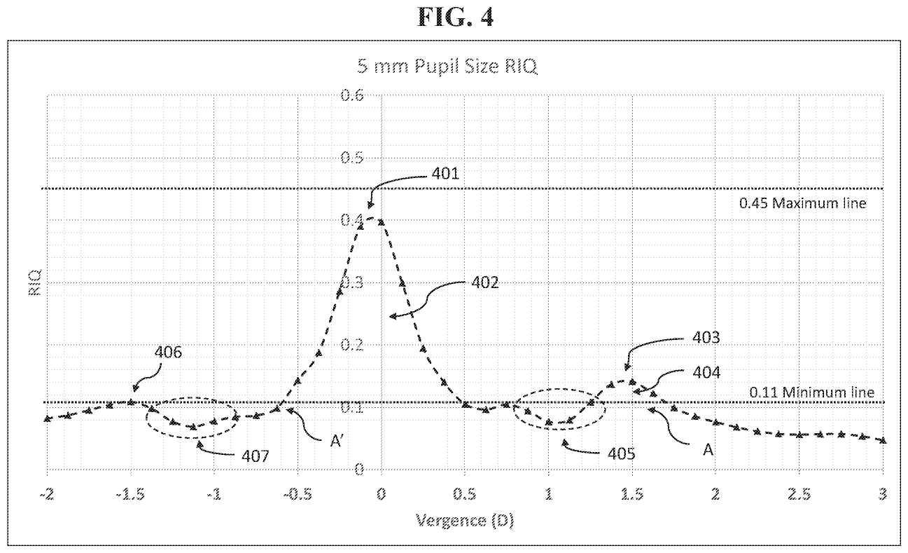

FIG. 4 is a plot of the through focus retinal image quality (RIQ) curve, in the form of the visual strehl ratio, over −2 D to +3 D vergences for the example lens described in FIG. 1 over a 5 mm pupil for a 589 nm wavelength. As illustrated, the through focus RIQ for the ophthalmic lens of FIG. 1 demonstrates an independent peak (denoted “primary peak” for the purpose of clarity) 401 that is approximately symmetrical around “0” vergence with a maximum RIQ value of about 0.4 and another independent peak (denoted “secondary peak” in specification and figures) 403 at about +1.5 D vergence with a maximum RIQ value of about 0.14. Additionally, the image quality may be further defined by calculating the area under the curve 402 at the primary peak 401, the primary Peak RIQ area, and the secondary peak 403, the secondary peak RIQ area 404. A maximum peak RIQ value may be defined as the highest value of the RIQ for the peak on the through focus RIQ curve. The peak RIQ area may be calculated as the area under the through focus RIQ curve bounded by the maximum RIQ value and a minimum line corresponding to an RIQ value of 0.11. The through focus RIQ curve shown in FIG. 4 for example lens of FIG. 1 may have a secondary peak RIQ value 403 above 0.11 that is independent because the RIQ values 405 immediately preceding the peak RIQ value 403 fall below 0.11 for a range of vergences of about 0.5 D at 405 (e.g., on side of the peak 403 with the lower vergence) and then rise above the 0.11 line to form the secondary peak RIQ value at 403. In contrast, the RIQ value at −1.5 D vergence (406) may not be considered a secondary peak RIQ value because the RIQ value remains below about 0.11 even though the values over region 407 (e.g., on side of the ‘peak’ at 406 with the lower vergence) remain below 0.11. In some embodiments, the through focus RIQ curve for a lens may have one or more peaks

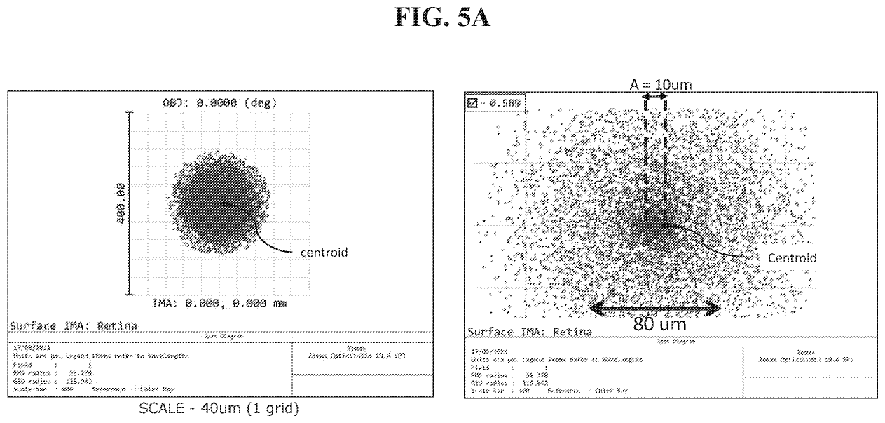

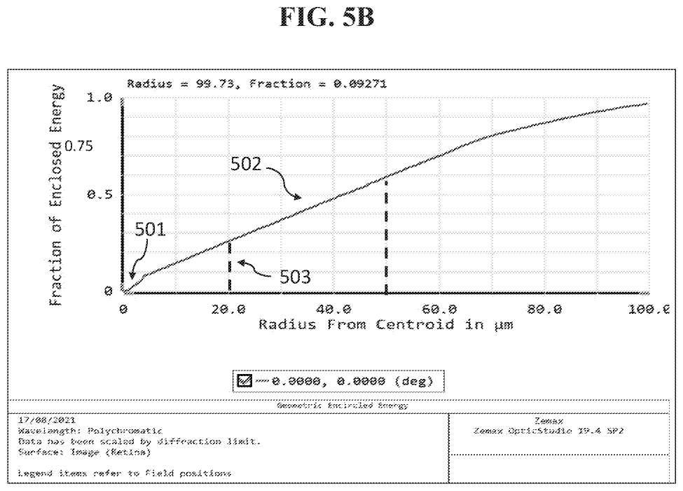

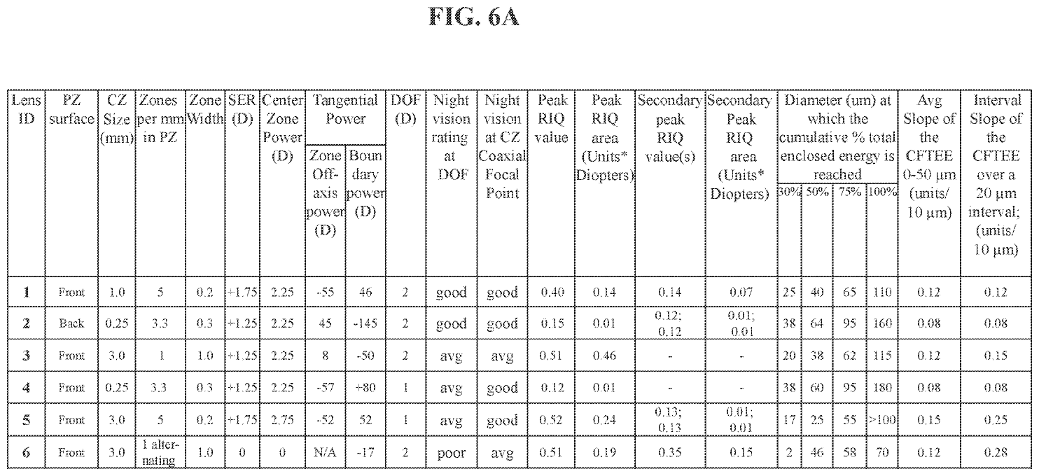

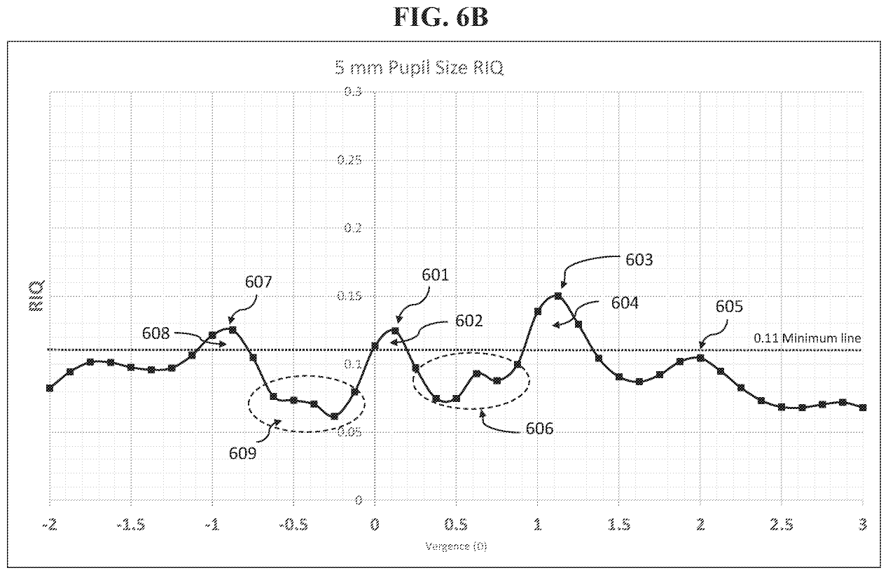

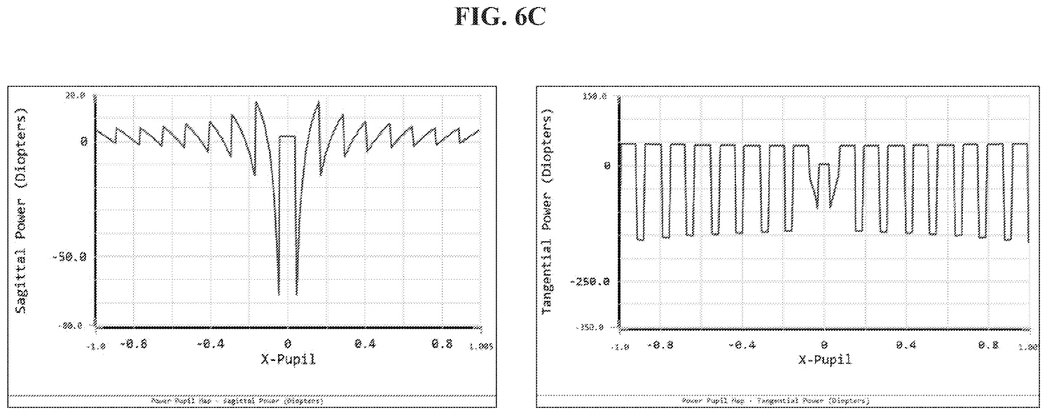

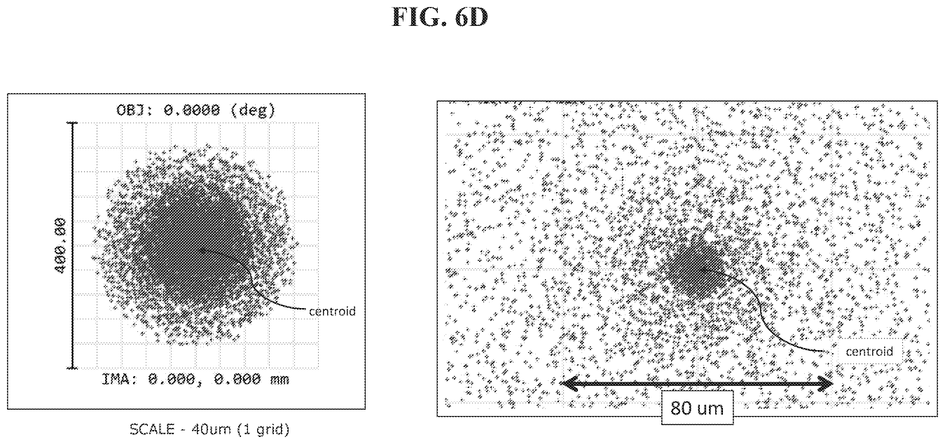

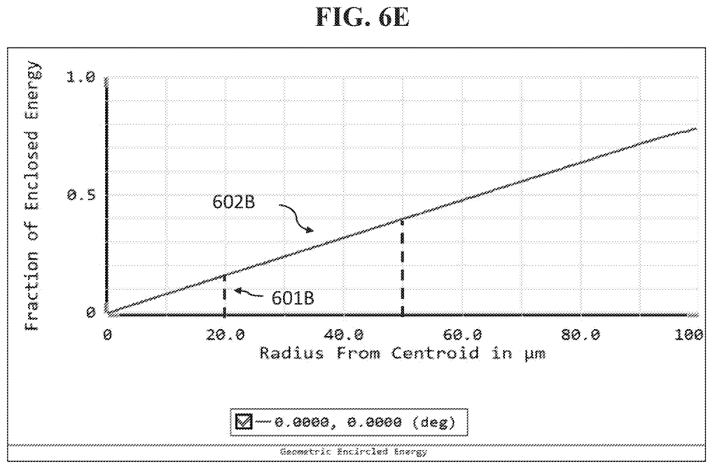

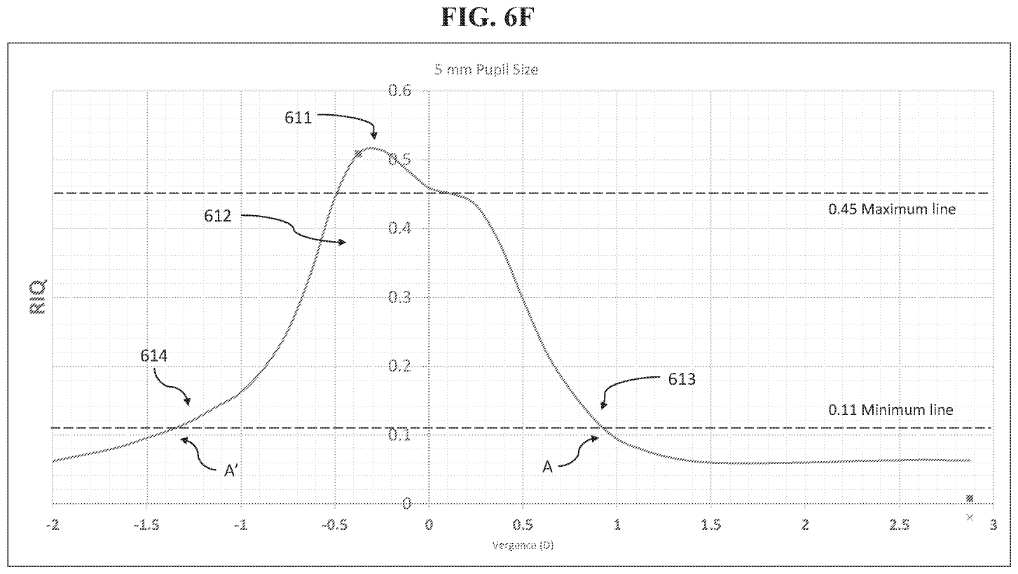

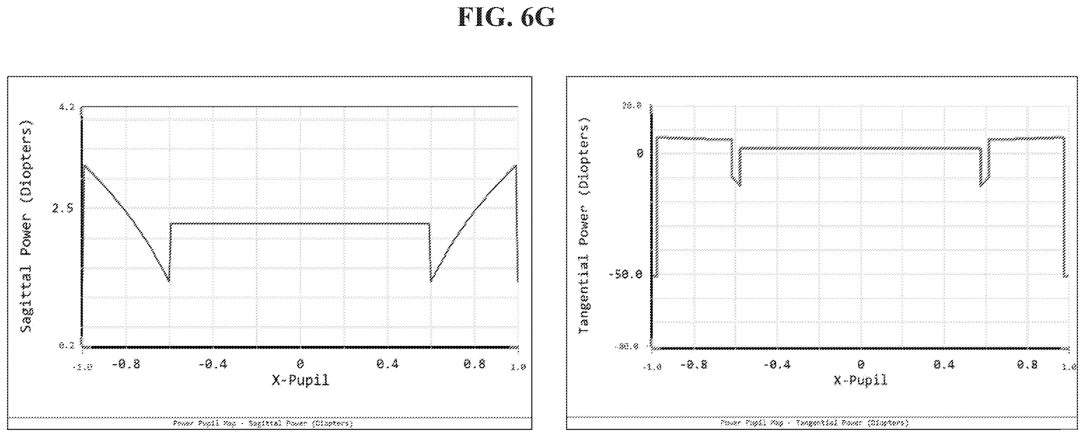

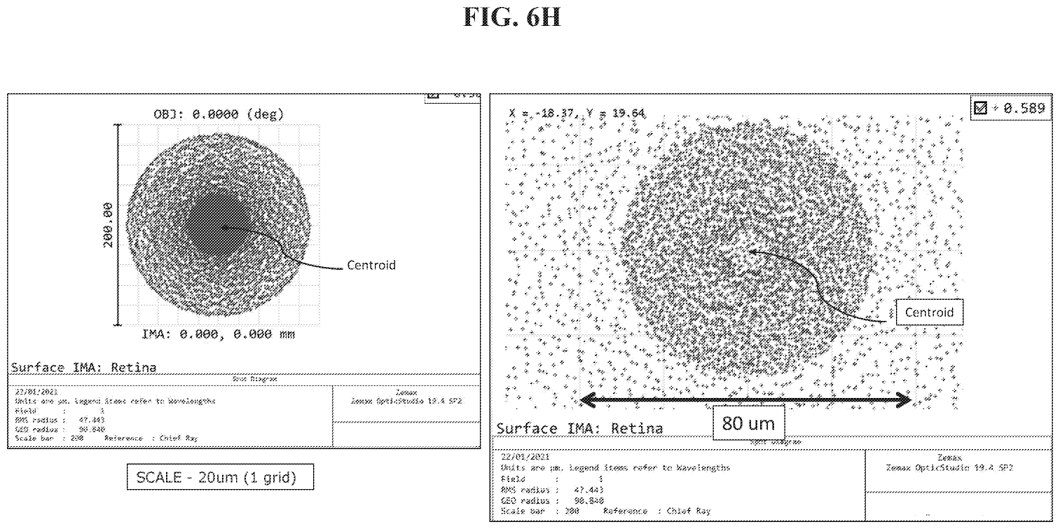

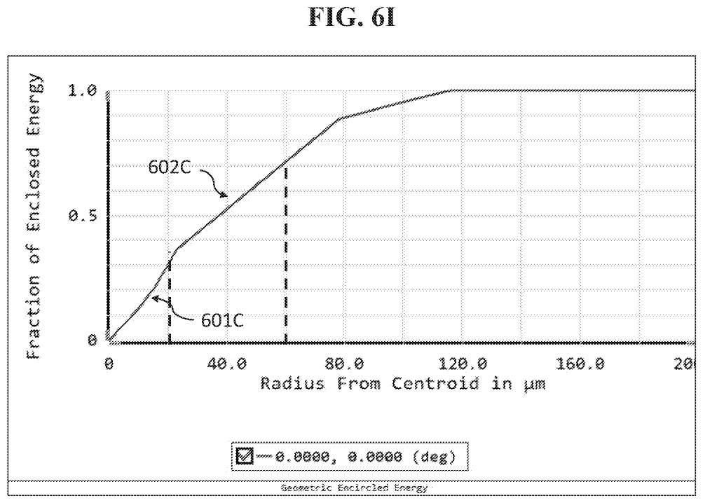

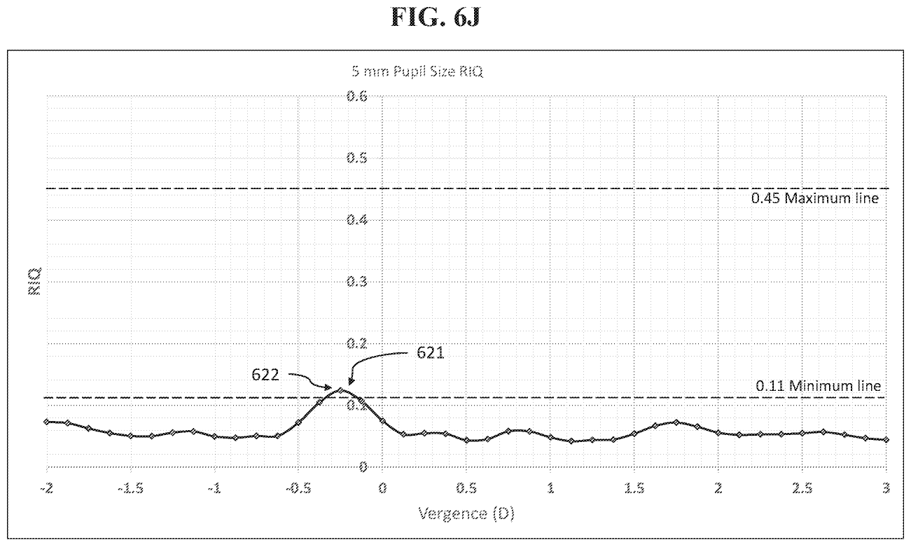

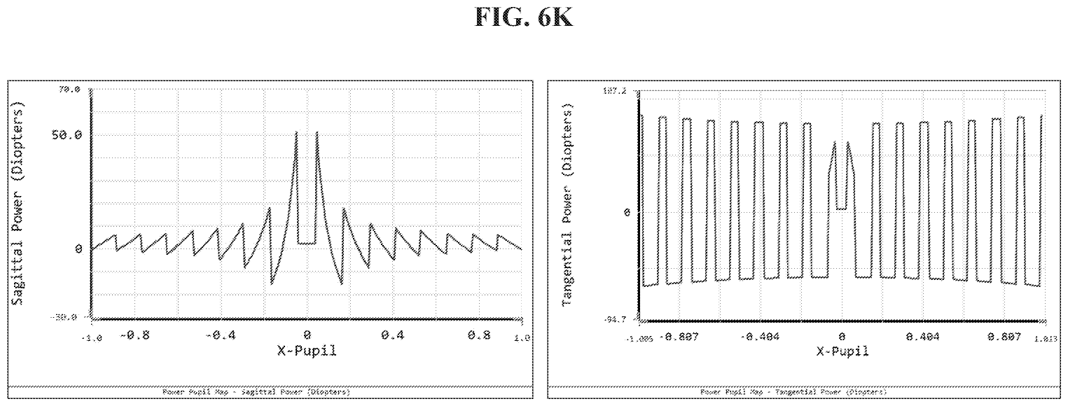

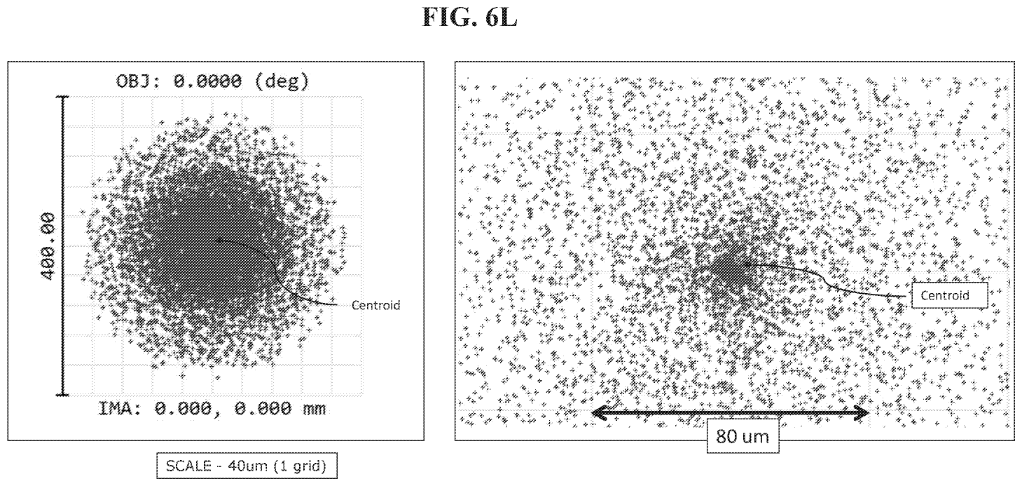

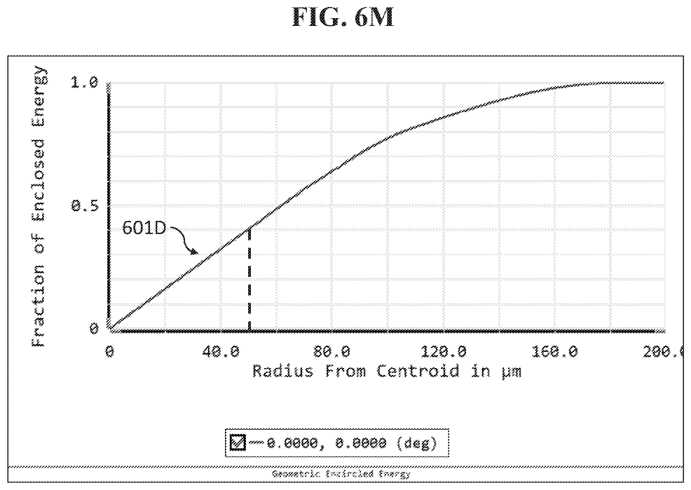

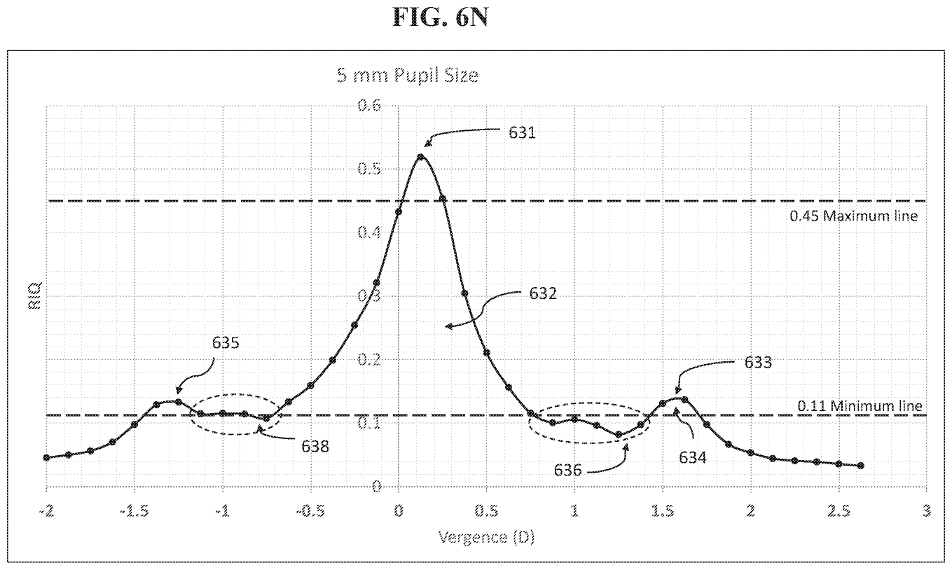

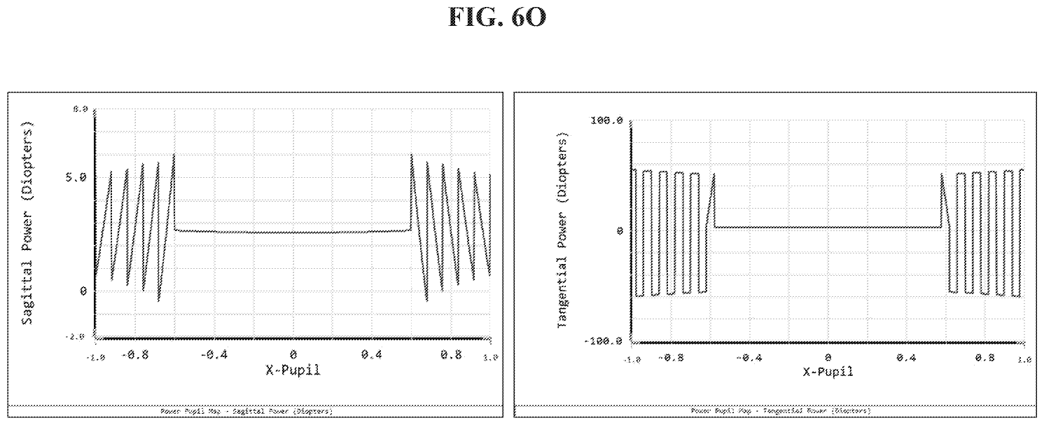

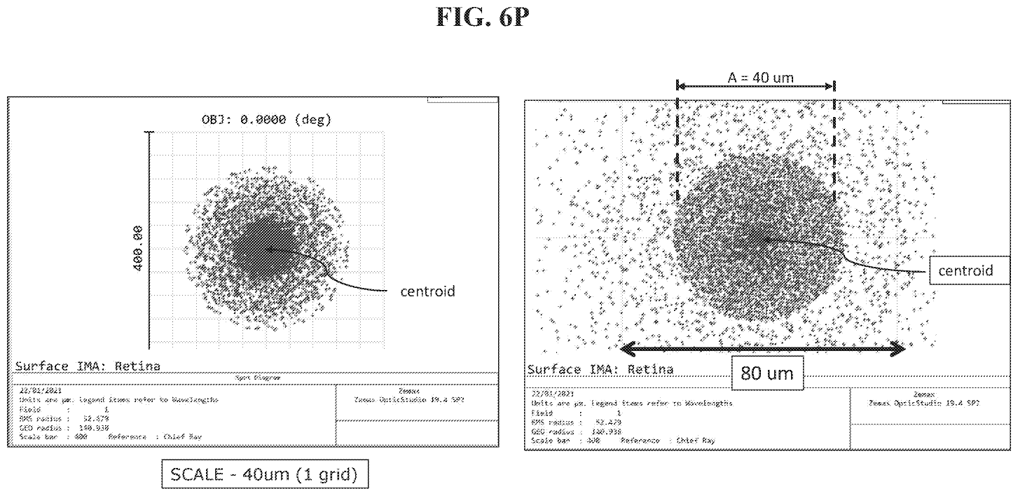

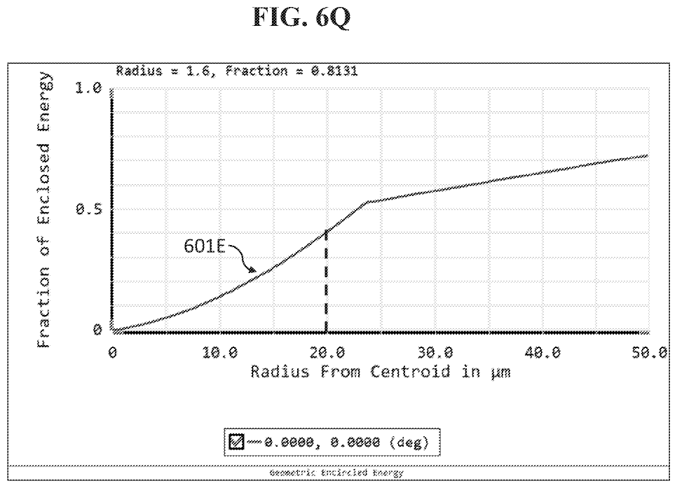

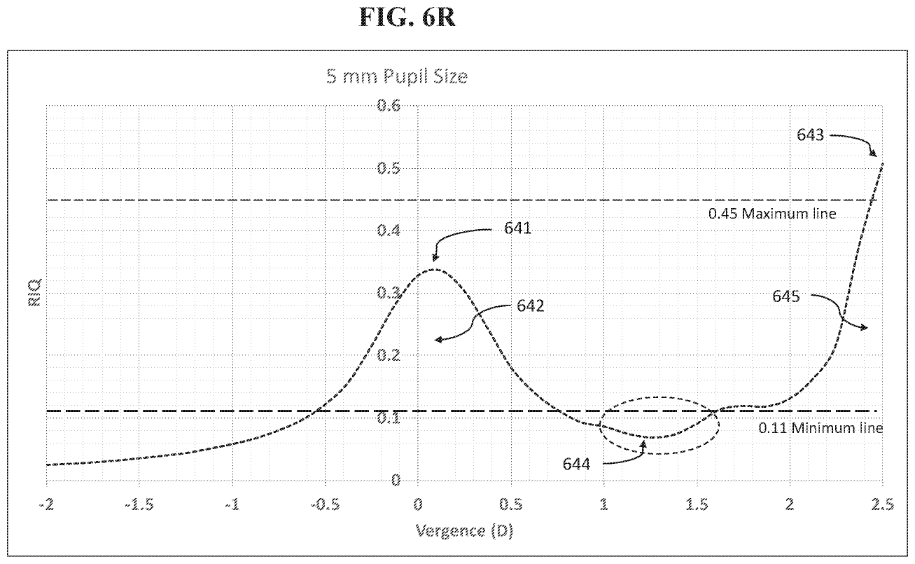

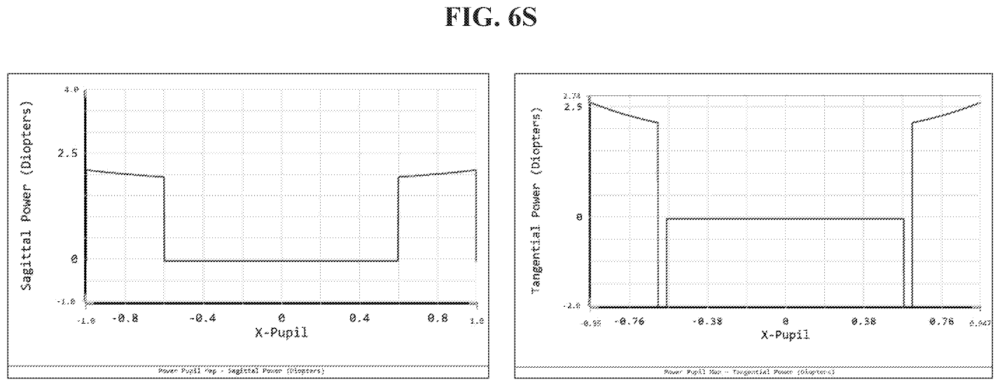

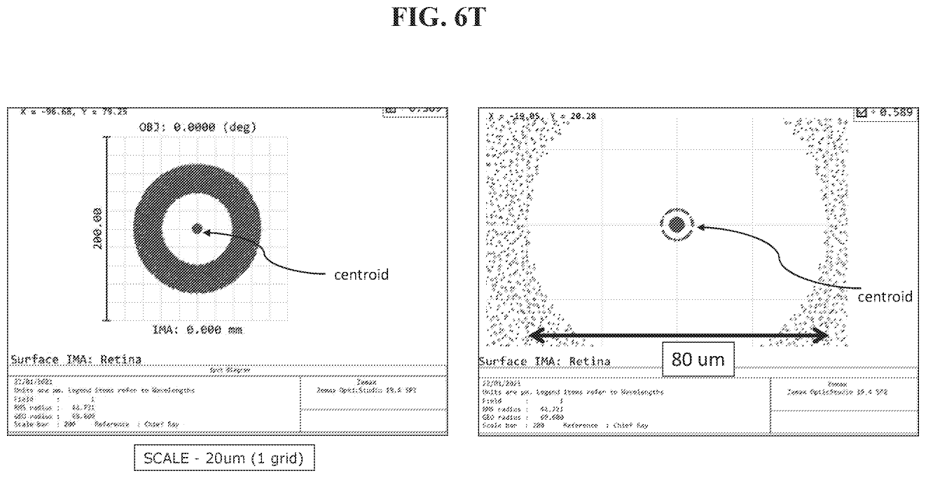

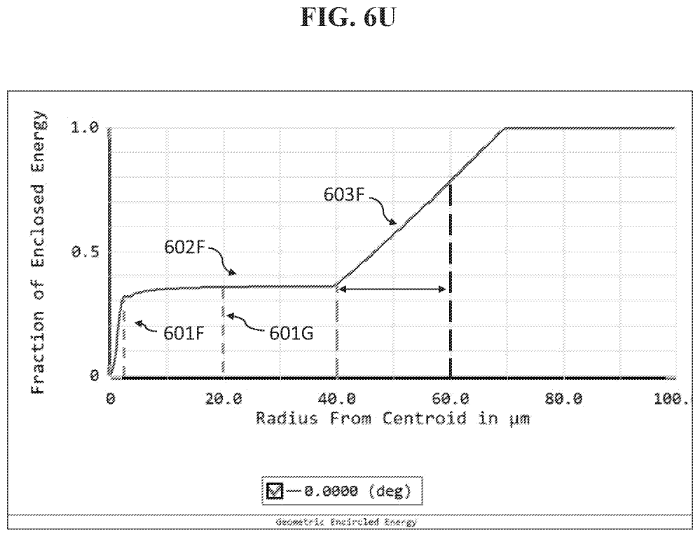

The distribution of the light energy across an image plane at a single vergence, e.g. at the retinal image plane, may be modeled qualitatively as a distribution of light rays across the retinal spot diagram in optical ray tracing software (e.g., Zemax) and may also be quantified by one or more metrics such as the total enclosed energy (e.g., the geometric encircled energy graph computed using ray-image surface intercepts and calculating the amount of the incident light energy over half chord distance in the optical system). FIG. 5A shows the distribution of light rays (dots) over the retinal spot diagram as modeled in optical design software (e.g., Zemax) for the ophthalmic lens embodiment of FIG. 1, and FIG. 5B is a plot of the cumulative fraction of total enclosed energy (CFTEE) over the half chord of the retinal spot diagram shown in FIG. 5A. The vergence, and therefore image plane, at which the spot diagram and CFTEE may be computed for the example lens of FIG. 1 may depend on the prescribed power of the center zone and may be prescribed relatively more positive in power than the distance spherical equivalent refractive error, SER, (center zone focal point 212a, FIG. 2B) e.g, about +0.5 D more positive than the SER, to provide the depth of focus (e.g. 216, FIG. 2B about fully anterior to the retinal image plane (214 as detailed in FIG. 2B). Therefore, as prescribed, the retinal image plane of the example lens of FIG. 1 may correspond to a vergence of about −0.5 D on the through focus RIQ curve of FIG. 4 and the retinal spot diagram and CFTEE shown in FIG. 5A, 5B may be computed at the retinal image plane at a vergence of about −0.5 D. Lens ID 6 is a bifocal contact lens design and the center zone may be prescribed as about the same as the SER and so the retinal image plane corresponds to about 0 vergence (FIG. 6R, 6T, 6U). As seen qualitatively from the lower (400 μm grid) scaled and higher (80 μm grid) scaled spot diagrams of FIG. 5A, the light rays formed at the retinal image plane (about −0.5 D vergence) may be seen as evenly distributed (e.g., with no regions of tightly packed or concentrated light rays outside of the small centroid). Likewise, the total enclosed energy plot in FIG. 5B shows the average slope 502 of the CFTEE progressing smoothly, without any rapid change in slope over any half chord intervals across the spot diagram with about 50% of the total enclosed energy accumulating before and after 40 μm from the centroid with an average slope of 0.12 units/10 μm. A less steep slope may indicate the absence of regions of concentrated light rays in the spot and regions of concentrated light rays may result in more relatively greater light energy that may increase the visibility of night visual disturbances such as glare, haloes and/or starbursts. Therefore, a useful metric of the evenness and uniformity of the distribution of light energy across the retinal image plane may be represented by the average slope of the CFTEE over a selected half chord from the centroid and/or any portion i.e. interval (the interval slope), along the half chord diameter of the spot diagram, for example, over any 20 μm or 30 μm or 40 μm or 50 μm or more of the half chord diameter from the centroid 501, over which about 30% or about 50% or about 75% of the CFTEE of the spot diagram may be spread.

The example lens of FIG. 1, may have a substantially smooth slope of about 0.12 enclosed energy units/10 μm across either a 40 μm half chord, or 50 μm half chord or 60 μm half chord of the spot diagram and/or about 50% of the total enclosed energy falling beyond about the first 40 μm half chord of the spot diagram, and the interval slope (over any 20 μm interval) was not greater than about 0.13 units per 10 μm confirming the qualitative observation from FIG. 5A that the light rays distributed across the retinal image plane may be substantially evenly distributed.

Further clinical observations with the ophthalmic lens embodiment of FIG. 1 in an eye with advanced presbyopia found good visual acuity and minimal ghosting over an extended range from far to near distances and indicates that the retinal image quality may be sufficient for good and/or acceptable vision. In addition, it was observed that the ophthalmic lens of FIG. 1 may also reduce, mitigate, or prevent one or more night vision disturbances that may accompany use of ophthalmic devices, systems and/or methods that incorporate simultaneous multifocal optics and/or an extended depth of focus. Clinical observations with the example ophthalmic lens embodiment of FIG. 1 in eyes corrected for the distance refractive error, as may occur, for example, in a non presbyopic accommodating eye, has also determined the retinal image quality provided may be sufficient to provide good distance vision (e.g., distance and near visual acuity and minimal ghosting) and may allow the extended depth of focus falling in front of the retina to be used for vision treatments, for example, of myopia progression and/or binocular vision disorders and/or visual fatigue syndromes e.g., computer vision syndrome. In addition, it was observed that the ophthalmic lens of FIG. 1 may also reduce, mitigate or prevent one or more night vision disturbances such as glare, haloes and/or starbursts that accompany the use of other ophthalmic devices, systems and/or methods that incorporate simultaneous multifocal optics and/or extended depth of focus for these other applications.

In some embodiments, the central zone and the plurality of narrow optical zones in the peripheral zone in combination with the front surface curvature, lens thickness, back surface curvature and the refractive index may be configured to form a power profile across the central and peripheral zones such that the lens may form on-axis focal points and off-axis focal points over a substantially wide range of vergences to provide an appropriate range of on-axis image qualities and/or light energy distributions along the optical axis and across the retinal image plane that may correct/treat the refractive condition of the eye by extending the depth of focus along the optical axis at least in part on and/or in front of the retina of the eye as well as to reduce, mitigate or prevent one or more night vision disturbances that accompany the use of such ophthalmic devices. In some embodiments, light rays from the central zone form a focal point that may have a higher light energy relative to focal points formed by light rays from the plurality of narrow annular optical zones of the peripheral zone. In some embodiments, the higher light intensity rays may not be positioned at about the midpoint of the most anterior and most posterior (e.g., retinal) image planes (e.g., at another position other than the mid-point of the depth of focus). In some embodiments, the higher light intensity rays may be positioned at about the midpoint of the most anterior and most posterior (e.g. retinal) image planes (e.g., at the mid-point of the depth of focus). In some embodiments, the light distribution across the image planes formed along the depth of focus may be substantially evenly distributed. In some embodiments, light rays from the plurality of narrow annular zones may have a lower light intensity that may have a reduced or lower interference on the near, intermediate, and/or distant image planes used for vision correction and/or vision treatment and may result in improved vision. In some embodiments, the interference from light rays distributed from the plurality of narrow optical zones across the anterior most image plane from retina may be less than the interference across the posterior most (e.g., retinal) image plane. In some embodiments, the light energy distributed at image planes along the optical axis and across the corresponding image planes may reduce, or mitigate, or prevent one or more night vision disturbances. In some embodiments, the center zone diameter and/or the power profile may be used to provide a preferred condition to minimize light interference on in-focus images by out-of-focus images and/or to reduce, or mitigate, or prevent one or more night vision disturbances (e.g. on-axis and/or off-axis focal points and image plane locations, light energy levels, image qualities, total enclosed energy distributions, and/or depth of focus). In some embodiments, the number of narrow optical zones and/or width and/or sagittal power profile and/or tangential power profile and/or m and/or p component values and/or P-V value and/or curvature and/or lateral separation and/or spacing and/or surface location of the optical zones may be used to minimize light interference of in focus images by out of focus images and/or to provide an extended depth of focus and/or to reduce, or mitigate, or prevent one or more night vision disturbances such as glare, haloes and/or starbursts.

FIG. 6A summarizes selected lens geometrical parameters, optical modeling outputs and clinical categorization for a series of lens designs. The clinical observations are categorized as good (providing good vision and relatively low night visual disturbances), or average (providing relatively poorer vision and relatively more visible night visual disturbances (e.g., similar to that observed with commercial multifocal soft contact lenses).

As used in FIG. 6A, the following abbreviations and descriptors should be understood as follows:
PZ refers to the ophthalmic lens surface incorporating the peripheral optical zone.
CZ size refers to the central optical zone diameter.
Zones per mm refers to the number of narrow optical zones located in the peripheral optical zone for every millimeter of the peripheral optical zone.
Zone width refers to the width of the narrow annular zones in the peripheral optical zone.
SER refers to the spherical equivalent refractive error for a user of the ophthalmic lens.
Central zone power refers to the base power of the central optical zone.
Zone off axis power refers to the diopter power of a middle portion of the first narrow optical zone of the cyclical power profile in the tangential direction.
Boundary power refers to the diopter power in the tangential direction at the boundary between the first and second narrow optical zones resulting from the surface contour formed by an outer portion of the first narrow optical zone, the transition between the first and second narrow optical zones and an inner portion of the second narrow optical zone.
DOF refers to the vergence range in diopters where a useful vision correction may be obtained for advanced presbyopia as determined from clinical observations.
Night vision ratings at DOF refers to ratings of night vision disturbances when the base power profile of the central optical zone is prescribed to position the DOF anterior to the retinal image plane starting from the retinal image plane (i.e., more positively powered than the central optical zone base power).
Night vision ratings at CZ focal point refers to ratings of night vision disturbances when the base power profile of the central optical zone is prescribed to correct the SER and thereby positioning a portion of the DOF both anterior and posterior to the retinal image plane.

FIGS. 6B, 6C, 6D, 6E, 6F, 6G, 6H, 6I, 6J, 6K, 6L, 6M, 6N, 60, 6P, 6Q, 6R, 6S, 6T, and 6U provide optical modeling results for the example lens designs ID 2 to ID 6 including, i) through focus RIQ distributions, ii) cyclical power profile (sagittal and tangential directions), iii) retinal spot diagrams at low (e.g. 200 μm×200 μm or 400 μm×400 μm grids) and high scales illustrating spatial distribution of light rays at the retinal image plane and iv) a plot of the CFTEE over the retinal image plane. Similar optical modeling details for the lens labelled Lens ID 1 have been previously presented in FIGS. 3-5, as the ophthalmic lens of FIGS. 1-5 corresponds to Lens ID 1.

FIG. 6A and FIG. 6B-6E provide details of an exemplary embodiment (Lens ID 2) of an ophthalmic lens that provides an extended depth of focus for vision correction e.g., presbyopia and/or vision treatment e.g., myopia control and further improves night vision by reducing/minimizing one or more visual disturbances such as glare, haloes and/or starbursts. Similar to Lens ID 1, the ophthalmic lens of Lens ID 2 comprises a central zone power profile that is relatively more positively powered than the distance refractive error (the vergence at about −1 D corresponds to the retinal image plane), a peripheral zone with a plurality of conjoined annular zones with line curvatures; a cyclical power profile in the sagittal and tangential direction in the peripheral zone with the cycles incorporating a “m” and “p” component, wherein the cyclical power profile may be designed/modulated (e.g., by altering “m” and “p” components values and sequence, and/or power progression slopes and/or power progression shapes over a power cycle and/or between “m” and “p” components (e.g., linear, curvilinear or other shape), and/or off axis powers and/or boundary powers) to distribute the light energy across a substantially wide range of vergences along the optical axis to result in a retinal image quality within a desired limit of ranges and furthermore, to evenly distribute the light energy across the retinal image plane; and wherein the ophthalmic lens provides an extended depth of focus for vision correction and/or vision treatment and may further substantially improve night vision by reducing one or more visual disturbances. Compared to Lens ID 1, Lens ID 2 has a smaller central zone of about 0.25 mm diameter, a peripheral optical zone comprising 3.3 annular zones/mm and located on the back surface of the ophthalmic lens (FIG. 6A). Although the central zone power of both lens Lens ID 1 and ID 2 may be the same e.g., about +0.5 D to +1 D more positively powered than the distance refractive error (the vergence at about −0.5 D to −1 D therefore corresponds to the retinal image plane), the different configuration of Lens ID 2 (diameter of the central zone, width of the annular zone in the peripheral optical zone, the location of the zones on the back surface) may result in a cyclical power profile in the sagittal and tangential directions that may be different between the lenses with varying “m” and “p” components (FIG. 6C) and FIG. 3). Lens ID 2 may a have a primary independent RIQ peak 603 and two secondary RIQ peaks 601 and 607 that may be independent because the portion of the RIQ curve immediately preceding the RIQ peak values 606 and 609 (e.g., on at least the side of the RIQ peak with the lower vergence) fall below the minimal RIQ value 0.11 (e.g., on at least one side of the RIQ peak with the lower vergence). The maximum RIQ value 603 for the primary RIQ peak at about +1.2 D vergence (located at an image plane in front of the retinal image plane) may be lower for Lens ID 2 than Lens ID 1 (about 0.15 versus about 0.4; compare FIG. 6B and FIG. 4) but the maximum RIQ value of any secondary independent peaks 601, 607 (FIG. 6B) and 401 (FIG. 4) formed for Lens ID 2 and ID 1 may be about the same. The RIQ areas (604, 602 and 402, 404) corresponding to the respective RIQ peak values for Lens ID 2 and Lens ID 1, respectively were calculated at about 0.01, 0.01 and 0.01 units*D for Lens ID 2 and 0.14 and 0.07 units*D for Lens ID 1 (FIG. 6A). Both lenses (ID 1 and 2) provide good vision with a range of depth of focus of about 2 D indicating that a RIQ value for a primary and secondary RIQ peaks in the range of about 0.11 to about 0.45 and RIQ areas in the range of about the levels calculated for ID Lens 1 and 2 may be adequate for user satisfaction and furthermore, the low light energy may minimize night visual disturbances compared to simultaneous vision lenses. FIG. 5A and FIG. 6D illustrating retinal spot diagrams for Lens ID 1 and Lens ID 2 indicate the distribution of light rays for both lenses to be substantially similar across the retinal spot diagram and this may be confirmed quantitatively by the CFTEE plots (FIG. 5B and FIG. 6E) where the average slopes 502, 602B were about 0.12 units/10 μm and 0.08 units/10 μm for Lens ID 1 and Lens ID 2 respectively. The interval slopes 503, 602C for Lens ID 1 and Lens ID 2 were about 0.12 units/10 μm and 0.08 units/10 μm, indicating that the slopes were smooth and constant and where 50% of the CFTEE fell beyond about 40 μm from the centroid for both lens types (FIG. 6A).

FIG. 6A and FIGS. 6F-6I provide details of another exemplary embodiment (Lens ID 3) of an ophthalmic lens that may provide similar extended depth of focus as Lens ID 1 for vision correction and/or vision treatment but may not substantially minimize the one or more night vision disturbances. Similar to Lens ID 1, the ophthalmic lens of Lens ID 3 comprises a central zone power profile that is relatively more positively powered (e.g. +1 D) than the distance spherical equivalent distance refractive error (the vergence at −1 D corresponds to the retinal image plane), a peripheral zone with a plurality of annular conjoined zones of a frequency of 1 zone/mm and formed with curves; a cyclical power profile in the sagittal and tangential directions in the peripheral zone with the cycles incorporating a “m” and “p” component, wherein the cyclical power profile in at least a sagittal direction may be designed/modulated (e.g., by altering “m” and “p” components values and sequence, and/or power progression slopes and/or power progression shapes over a power cycle and/or between “m” and “p” components (e.g., linear, curvilinear or other shape), and/or off axis powers and/or boundary powers) to provide an extended depth of focus for vision correction and/or vision treatment. However, unlike Lens ID 1, Lens ID 3 may not distribute (or at least not distribute as effectively) the light energy along the optical axis and/or across the retinal image plane within value range limits to reduce/minimize night vision disturbances from glare, haloes and/or starbursts. Compared to Lens ID 1, Lens ID 3 may have a larger central zone of 3.0 mm diameter and a peripheral optical zone comprising 1.0 annular zones per mm of the lens and the design e.g. surface curvature configuration located on the front surface of the ophthalmic lens (FIG. 6A). Although the central zone power and the resulting extended depth of focus may be about the same, the different configuration (e.g., diameter of the central zone, width of the annular zone in the peripheral optical zone, the surface curvature and/or the location of the zones on the front surface) may result in a power profile, including a cyclical power profile in the sagittal and tangential directions that may be different between the lenses with, for example, varying “m” and “p” components and/or off axis powers and/or boundary powers (FIG. 6H and FIG. 3). Although the depth of focus for both lens examples may be about 2 D (FIG. 6A), the through focus RIQ curve for Lens ID 3 (FIG. 6F) may be substantially different to the through focus RIQ curve for Lens ID 1 (FIG. 4). Lens ID 3 forms single peak RIQ 611 with a maximum peak RIQ value for the primary peak at about “0” vergence (an image plane about +1 D in front of the retinal image plane) may be higher for Lens ID 3 than Lens ID 1 (about 0.52—FIG. 6F) versus about 0.4, FIG. 4) and the through focus RIQ curve for Lens ID 3 may remain high over a broader range of vergences over about 2 D depth of focus as seen at 613 to 614 (FIG. 6F) to provide a useful vision correction over the depth of focus. In contrast, as previously described in FIG. 4, Lens ID 1 may form 2 peaks including a primary peak 401 with a maximum peak value of about 0.4 at “0” vergence, the spread of the primary peak being narrow over a smaller range of vergences from a −0.6 D to +0.5 D and a secondary independent peak 403 with a maximum peak RIQ value of about 0.14 and spread over a vergence from +1.25 D to +1.7 D. Clinical observations (FIG. 6A) indicate that both Lens ID 1 and ID 3 provide good vision for a range (depth of focus) of about 2 D and this may be consistent with the finding that the RIQ values at about the ends of the depth of focus e.g. between about A and A′ on the curve may be about similar for the lens types. As previously noted with Lens ID 2, despite the low maximum peak RIQ values, good vision correction may be achieved along the extended depth of focus range. However, unlike Lens ID 1 and ID 2, Lens ID 3 does not appear to minimize night vision disturbances with performance possibly similar to night vision disturbances observed with regular simultaneous vision multifocals (FIG. 6A). The area under the curve for the primary RIQ peak 611 (the Peak RIQ Area 612) of Lens ID 3 (FIG. 6F) was about 0.46 units×D and substantially greater than the area under the curve 402 for primary RIQ peak 401 of Lens ID 1 at about 0.14 units×D. The relatively higher image quality of Lens ID 3 distributed over a broader range of vergences may provide a more intense and concentrated light energy at the retinal image plane and may result in substantially greater night visual disturbances than Lens ID 1. FIG. 5A and FIG. 6H illustrating plots of the retinal spot diagrams for Lens ID 1 and Lens ID 3 indicate the distribution of light rays for both lenses and highlight the relatively less spatially uniform distribution of light rays across the retinal spot diagram for Lens ID 3 and confirmed quantitatively in the CFTEE plots (FIG. 5B and FIG. 6I) where the average slope of the CFTEE over the 50 μm half chord 602C for Lens ID 1 was 0.12 units/10 μm (FIG. 6A) and the interval slope 601C over the half chord between the centroid and 20 μm was significantly steeper for Lens ID 3 than Lens ID 1, 503, (0.15 units/10 μm vs 0.12 units/10 μm). Significantly, within the first 20 μm half chord of the retinal image spot diagram, the fraction of the total enclosed energy accumulated was greater at 35% for Lens ID 3 (FIG. 6I) versus 20% for Lens ID 1 (FIG. 5B).

FIGS. 6J-6M and 6N-6Q provide details of two other exemplary embodiments of ophthalmic lenses (Lens ID 4 and Lens ID 5, Table in FIG. 6A) where lens ID 4 may comprise a substantially smaller central zone of 0.25 mm diameter with a power profile that is relatively more positively powered (e.g. about +1 D) than the distance spherical equivalent refractive error (the vergence at about −1 D corresponds to the retinal image plane) of a user, a peripheral zone with a plurality of conjoined annular zones with line curvatures and about 3.3 annular zones/mm, a cyclical power profile in the sagittal and tangential directions in the peripheral zone with the cycles incorporating a “m” and “p” component wherein the cyclical power profile at least in a sagittal direction may be designed/modulated (e.g., by altering “m” and “p” components values and sequence, and/or power progression slopes and/or power progression shapes over a power cycle and/or between “m” and “p” components (e.g., linear, curvilinear or other shape), and/or off axis powers and/or boundary powers) to distribute the light energy across a substantially wide range of vergences along the optical axis to result in a retinal image quality of a desired range and furthermore, to evenly distribute the light energy across the retinal image plane; and wherein the ophthalmic lens provides an extended depth of focus for vision correction and/or vision treatment. The peripheral optical zones of Lens ID 2 may be formed on the back surface whereas those of Lens ID 4 may be formed on the front surface (FIG. 6A). Although the central zone power profile may be about the same, the different configuration (e.g. line curvature on front versus back surfaces) may result in a cyclical power profile in the sagittal and tangential directions that is different between the lenses with, for example, varying “m” and “p” components, off axis powers and/or boundary powers (FIG. 6C and FIG. 6K) and the resultant clinically observed depth of focus different between the embodiments, with over about 2 D versus 1 D for Lens ID 2 and Lens ID 4 respectively (FIG. 6A). The through focus RIQ curves of lenses ID 2 and ID 4 (FIG. 6B and FIG. 6J), respectively) show very low RIQ values of about 0.15 or less across all vergences. In the embodiment Lens ID 2, three independent (RIQ values in regions 606, 609 on lower vergence side of the RIQ peak less than 0.11) peak RIQ values 601, 603 and 607 (FIG. 6B; regions 606, 609) may be formed with maximum peak RIQ values above about 0.11 and, as reported in FIG. 6A, Lens ID 2 provides good vision over the depth of focus e.g. for advanced presbyopia. In contrast, the through focus RIQ curve for Lens ID 4 (FIG. 6J) illustrates a single primary peak 621 with maximum RIQ of about 0.12 at about −0.2 D vergence (at an image plane about +1 D more anterior to the retinal image plane); at the remaining vergences, the maximum RIQ is below about 0.11 and due to the RIQ being very low and as noted in FIG. 6A, clinically the lens was unable to provide good vision along an extended range as with Lens ID 2.

As summarized in FIG. 6A, ID Lenses 1 to 3 may provide good vision correction over a depth of focus of about 2 D and the lenses may provide peak RIQ values for the through focus curve over the range of vergences illustrated of at least about 0.11 or greater (FIGS. 4, 6B, 6F). In comparison, the RIQ values for ID Lens 4 were almost entirely below about 0.11 across the range of vergences illustrated and thus may not have been sufficient image quality to provide good vision and therefore, it may appear that a maximum peak RIQ value substantially lower than expected of at least about only 0.11 may be required to provide good vision correction. However, only Lens ID 1 and 2 but not Lens ID 3, may minimize night vision disturbances as the RIQ values at one or more peaks along the through focus RIQ curve may be relatively low, at about 0.45 or lower, and the corresponding peak RIQ areas for one or more maximum RIQ peaks may also be balanced at about 0.14 units×Diopters (FIG. 6A). These peak RIQ areas for Lens ID Lens 1 and 2 were substantially lower than the peak RIQ area 612 for Lens ID 3 of 0.46 units×Diopters and these differences may also be reflected in the light energy distribution across the retinal image plane (e.g. CFTEE) where, compared to Lens ID 3, Lens ID 1 and 2 produced a more spatially uniform light energy distribution with an interval slope of the CFTEE over 20 μm (503 and 601C, FIGS. 5B and 6I, respectively) of no greater than about 0.13 units/10 μm (FIG. 6A) compared to Lens ID 3 at about 0.15 units/10 μm indicating a significant concentration of energy over a portion of the spot diagram even though Lens ID 1 and 3 had 50% of the CFTEE distributed over the 40 μm half chord of the retinal spot diagram. Based on these values, it may be expected that Lens ID 4 may also minimize night vision disturbances compared to typical simultaneous vision multifocals based on the relatively low peak RIQ values 621 (about 0.12, FIG. 6J) and corresponding RIQ area 622 (about 0.01 units×Diopters, FIGS. 6J, 6AJ), relatively uniform distribution of light rays modeled across the retinal spot diagram (FIG. 6K) and confirmed quantitatively by the CFTEE plots in FIG. 6M where about 50% of the total enclosed energy fell beyond 60 μm from the centroid of the retinal spot diagram and the average slope 601D of the CFTEE curve was not steep at about 0.08 units/10 μm over the 50 μm interval (FIG. 6M). However, night vision disturbances with Lens ID 4 was observed to be similar to other simultaneous multifocals (FIG. 6A) because of the overall very low RIQ values, for example below 0.11, across most of the depth of focus through focus RIQ curve provided a lens with overall lower/poor image quality generally including that may also contribute to night vision disturbances.

FIG. 6A and FIG. 6N-6Q provide details of another exemplary embodiment (Lens ID 5) with a peripheral zone configured substantially similarly to Lens ID 1 to provide an extended depth of focus range for vision correction and/or vision treatment. Similar to Lens ID 1, the ophthalmic lens of Lens ID 5 comprises a central zone power profile that is relatively more positively powered (e.g., about +1 D) than the distance spherical equivalent refractive error (the vergence at about −1 D corresponds to the retinal image plane), a peripheral zone with a plurality of conjoined annular zones with line curvatures and formed on the front surface of the ophthalmic lens; a cyclical power profile in the sagittal and tangential directions (FIG. 6O) in the peripheral zone with cycles incorporating a “m” and “p” component, wherein the cyclical power profile at least in a sagittal direction may be designed/modulated (e.g., by altering “m” and “p” components values and sequence, and/or power progression slopes and/or power progression shapes over a power cycle and/or between “m” and “p” components (e.g., linear, curvilinear or other shape), and/or off axis powers and/or boundary powers) to distribute the light energy across a substantially wide range of vergences along the optical axis to result in a retinal image quality within a desired limit ranges and furthermore, to evenly distribute the light energy across the retinal image plane; and wherein the ophthalmic lens provides an extended depth of focus for vision correction and/or vision treatment and may further substantially improve night vision by reducing one or more visual disturbances. Lens ID 5 has a substantially larger central zone of 3.0 mm diameter than Lens ID 1 (1.0 mm) but both lens types comprise a peripheral zone comprising narrow annular zones of similar width (0.2 mm or 5 cycles/mm) and consequently, Lens ID 5 may have fewer annular zones in the peripheral optical zone from its smaller width (FIG. 6A). Although the distance refractive error power and the narrow annular zones widths may be about the same, the cyclical power profiles in the sagittal and tangential directions formed in the peripheral optical zone and the extended depth of focus may be substantially different between the lenses because other geometrical configurations e.g., central optical zone diameters, the plurality of annular zones in the peripheral optical zone and the distance of the first of the annular zones from the optical axis may result in the different light energy distribution along the optical axis and along the retinal spot diagram between the two lens types. The through focus RIQ curve of lens ID 5 (FIG. 6N) shows an independent peak (denoted “primary RIQ peak” 631 for the purpose of clarity) at about +0.1 D vergence (e.g., an image plane more anterior to the retinal image plane by about +1 D) with a maximum peak RIQ value of 0.52 and is higher than the peak RIQ value 401 of Lens ID 1 (about 0.4, FIG. 4). Both lens types may form other independent peaks (denoted “secondary” peaks) at 633, 635 Lens ID 5, FIG. 6N and 403 Lens ID 1, FIG. 4 because of RIQ values in regions 636, 638 (FIG. 6N), 405 (FIG. 4) are about <0.11) with maximum peak RIQ values for these secondary peaks at about similar values (about 0.13). Additionally, the area under the curve or peak RIQ area 632 for Lens ID 5 is about 0.24 units×Diopters and substantially larger than the peak RIQ area 411 for Lens ID 1 (0.14 units×Diopters). Therefore, the light energy formed at the retinal image plane by Lens ID 5 may be significantly higher than Lens ID 2. As observed clinically, both lenses may provide good vision along the depth of focus of about 2 D demonstrating a relatively low level of RIQ of about 0.11 or above may be sufficient for user satisfaction. However, despite the similarities in the through focus RIQ curves for the majority of the vergences, clinical observations indicated that Lens ID 5 may not reduce/minimize night vision disturbances compared to commercially available multifocals because the RIQ areas 632 and 402 of the lens types (FIG. 6N and FIG. 4 for Lens ID 5 and ID 1, respectively) may be substantially different because the larger central zone of Lens ID 5 may substantially increase the light energy falling across the retinal image plane as compared to Lens ID 1. FIG. 5A and FIG. 6D illustrating plots of the retinal spot diagrams for Lens ID 1 and Lens ID 5 may indicate the distribution of light rays on the retinal image plane for both lenses and the relatively less spatially uniform distribution of light rays with increased concentration of light rays around the centroid for Lens ID 5 (diameter of A=40 μm, FIG. 6P) than Lens ID 1 (diameter A=10 μm, FIG. 5A). The CFTEE plots (FIGS. 5B and 6Q for Lens ID 1 and 5, respectively) also show the total enclosed energy formed calculated over the retinal spot diagram by Lens ID 5 was substantially more concentrated with nearly 50% of the total enclosed energy falling within about 20 μm of the centroid (interval slope 601E of about 0.25 units/10 μm over the 20 μm half chord diameter) compared to about 60 μm for Lens ID 1 and the interval slope 503 over 20 μm, of Lens ID 1 was less steep at about 0.12 units/10 μm (FIG. 6A). This difference in light energy distribution across the retinal image plane may, at least in part, contribute to the differences in night vision performances.

FIG. 6A and FIG. 6R-6U provide design and optical modeling results of an ophthalmic lens (Lens ID 6) e.g., a soft contact lens incorporating a simultaneous vision optical design used for vision correction e.g., presbyopia and/or vision treatment e.g., myopia control. The contact lens is an annular concentric optical design comprising a 3 mm center zone with a base power profile powered to correct the distance refractive error, a peripheral zone with four 1 mm wide annular zones with zones 1 and 3 providing more positive power than the center zone by +2D in the sagittal direction and zones 2 and 4 providing a power equal to the center zone base power (FIG. 6S). The center zone and the peripheral zones may be coaxial and form 2 focal points on the optical axis that may be non-cyclical (e.g., the power profile does not oscillate around the base power). The more positively powered annular zones of Lens ID 6 provide a vision correction of a close-up refractive error in presbyopia e.g., high addition presbyopia and/or a vision treatment defocus in an image plane anterior to the retinal plane in an accommodating progressing myope to control myopia progression. The through focus RIQ curve for the bifocal contact lens, Lens ID 6, plotted in FIG. 6R shows an independent peak (denoted “primary” RIQ peak) 643 at about +2.5 D vergence with a maximum RIQ peak value of 0.51 and RIQ area 645 of 0.46 units×D. An independent peak 641 (RIQ values at 644 below 0.11) (denoted “secondary” RIQ peak) at about +0.2 D vergence (located at the retinal image plane during distance vision) has a maximum RIQ peak value of 0.35 and RIQ area 642 of 0.19 units×D. The distribution of light rays across the retinal spot diagram modeled for Lens ID 6 in FIG. 6T indicates light rays are markedly concentrated to smaller regions across the retinal image plane. Likewise, the CFTEE curve for Lens ID 6 plotted in FIG. 6U quantifies the non-uniform distribution of light energy over the image plane, for example, about 35% of the light energy falling over the first 3 μm half chord from the centroid (601F) and then almost no additional energy accumulating between the 5 μm to 40 μm half chord interval 602F (e.g., zero slope) and the remaining 65% of the light energy concentrated over the 40 μm to 70 μm half chord interval (relatively steep interval slope 603F over 20 μm between 40 μm and 60 μm of about 0.28 units/10 μm).

As categorized in FIG. 6A, Lens ID 6 may provide compromised vision typical of simultaneous vision optical designs as the defocused images on the optical axis substantially (e.g., due to the peak RIQ value and peak RIQ areas) interfere with in focused images at the retinal image plane. Night vision was also observed clinically as average because the light rays may not be uniformly distributed across the retinal image plane (FIG. 6T), for example light energy concentrated in narrow regions (FIG. 6U) resulting in substantial disturbances to night vision by one or more visual disturbances such as glare, haloes and starbursts. The modeling results with Lens ID 6 in FIGS. 6R-6U indicate retinal image quality outside of a desired range e.g. RIQ peak values and peak areas outside the range of about 0.11 to about 0.45 and >0.16 units×D, respectively and may be too high for user satisfaction, and an interval slope 601G (FIG. 6U) of the CFTEE curve greater than about 0.13 units/10 μm over a 20 μm half-chord diameter that may promote night visual disturbances such as glare, haloes and/or starbursts compared to simultaneous vision lenses.

Therefore, from the various ophthalmic lenses (FIGS. 3, 4, 5, 6A-6U) designed with a range of geometrical parameters resulting in a range of optical properties and varying clinical observations, a series of criteria may be defined to design ophthalmic lenses with an extended depth of focus for vision correction and/or vision treatment as well as an improved night vision performance by reducing, mitigating and/or preventing one or more visual disturbances (e.g., by providing lower light energies).

An improved ophthalmic lens with an extended depth of focus for vision correction and/or vision treatment as well as an improved night vision performance by reducing, mitigating and/or preventing one or more visual disturbances may have one or more RIQ values at one or more peaks along the through focus curve be within an acceptable range e.g., an ‘acceptable’ peak RIQ value range is where the maximum peak RIQ value of one or more independent peaks is between about 0.11 and about 0.45. The peak RIQ values and peak RIQ areas outside the defined acceptable value ranges may be determined as ‘substantially unacceptable’ or “slightly unacceptable” as they may be too weak (if <about 0.11 maximum RIQ value) to provide good vision correction or too strong (if >about 0.45 maximum RIQ value) to provide a relatively uniform distribution of relatively low light energy across the retinal spot diagram, for example where the average slope of the CFTEE plot over the 50 μm half chord of the retinal spot diagram may be less than about 0.13 units/10 μm and/or where an interval slope over a 20 μm half chord is not greater than about 0.13 units/10 μm.

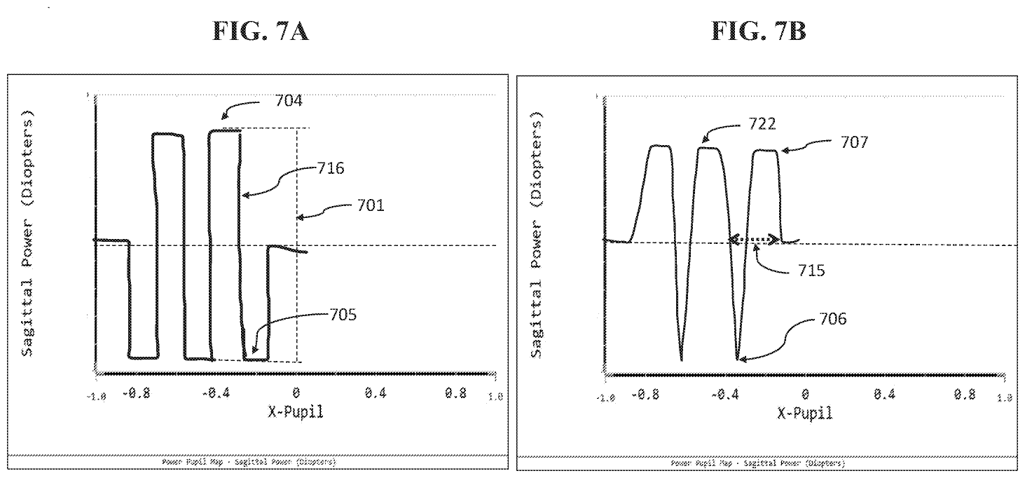

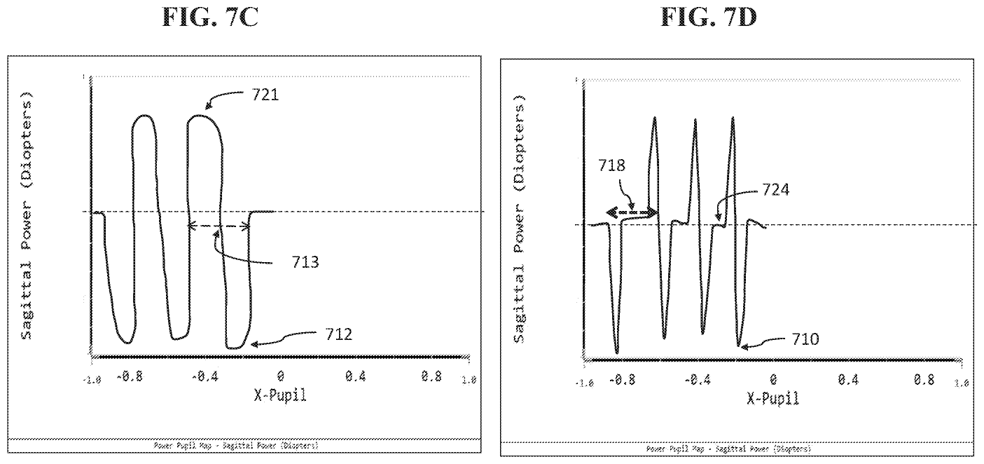

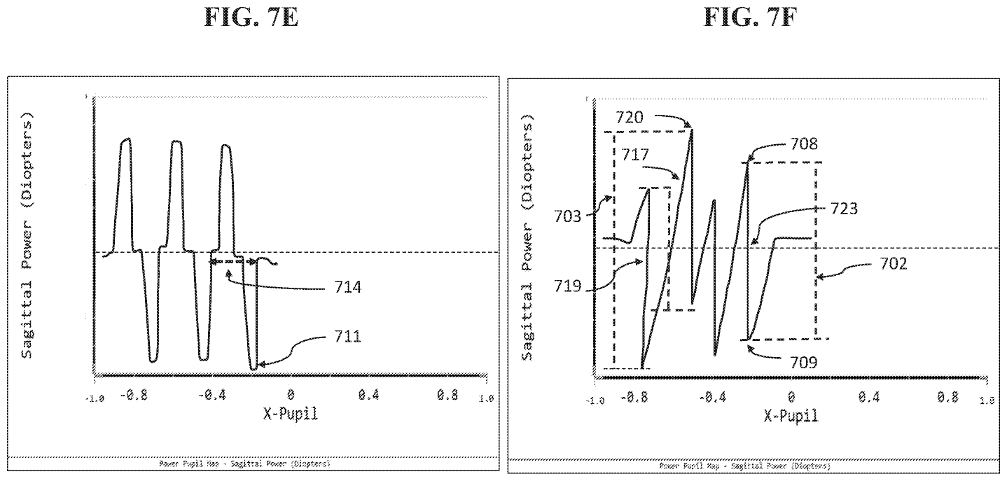

FIGS. 7A-7F provide schematic illustrations of different configurations of cyclical power profiles in the sagittal direction that may be produced by a plurality of optical zones incorporated into one or more of central and/or peripheral optical zones of ophthalmic lenses to provide extended depth of focus for vison correction and/or vision treatment and also reduce, mitigate and or prevent night vision disturbances such as glare, haloes and starbursts. The embodiments of 7A-7F may be configured to provide a light energy distribution across a wide range of vergences and to provide independent peak RIQ values and peak RIQ areas generated at vergences along the through focus RIQ curve and/or a light energy distribution over the retinal image plane to within the desirable limits disclosed herein. The pattern of the cyclical power profile pattern may be changed in several parameters, for example in the sagittal direction and as labelled in FIGS. 7A-7F including peak to valley (P-V) values of a cycle of the cyclical power profile may be the same or different e.g., 701 (FIG. 7A), 702, 703 (FIG. 7F), the value of the p and m components e.g., at 704 and 705 (FIG. 7A), 706 and 707 (FIG. 7B), 708 and 709 (FIG. 7F) and/or the order p and m components e.g., the m component first at 710 (FIG. 7D), 711 (FIG. 7E) or the p component first e.g., at 707 (FIG. 7B), the width of a single cycle e.g. a wider cycle at 713 (FIG. 7C) than the cycle at 714 (FIG. 7E) and/or an unbalanced cycle where a first portion of the cycle (above the base power line) may be wider than another portion of the cycle (below the base power line) e.g., at 715 (FIG. 7B) of the cyclical power profile, the slope of the power progression within a cycle may be steeply sloped e.g., at 716 (FIG. 7A) and steeper than a more sloped portion of a power profile cycle e.g., at 717 (FIG. 7F), may be constant in power (e.g., is not sloped over a portion of the power profile cycle) e.g., at 718 (FIG. 7D), or where the m component may not equal the p component e.g., p<m at 719 (FIG. 7F) or the where the power progression may change and/or transition within a cycle e.g., the transition at the peak or trough of a p and/or m component may be sharp e.g., at 720 (FIG. 7F), gradual at 721 (FIG. 7C) or slow (e.g., plateaus) at 722 (FIG. 7B) or where the power profile may progress over a portion of a zone e.g. at the base power where the cycle may not slow e.g., at 723 (FIG. 7F) or plateaus e.g., at 724 (FIG. 7D).

In some embodiments, the annular optical zones may comprise at least one cycle and the cycles may be located, at least in part, in the peripheral zone. In some embodiments, the frequency of power profile oscillations across the optical zone may be constant or may vary across the optical zones and may have a frequency defined as cycles/mm, for example, 0.5 cycles/mm, 1 cycles/mm or 1.5 cycles/mm or 2 cycles/mm or 5 cycles/mm or 10 cycles/mm or 20 cycles/mm or 50 cycles/mm or 100 cycles/mm or higher frequency. In some embodiments, the Peak to Valley (P-V) value of the cycles in a sagittal and/or tangential direction within an optical zone may be defined as the absolute power range between the ‘m’ and ‘p’ components. In some embodiments, the P-V value may be constant across the peripheral zone or may not be constant across the peripheral zone, for example, the P-V value may increase from the first optical zone to the last optical zone across the e.g., peripheral zone or may decrease from the first to the last optical zone across the e.g. peripheral zone or may not change in any pattern or may be random. In some embodiments, the P-V value in a sagittal and/or tangential direction may be very low e.g., be about 1 D or may be very high e.g., be about 600 D and/or anywhere in between. In some embodiments, the value and/or ratio of the m and p components in the sagittal and/or tangential direction may be constant over the optical zones or may decrease or increase toward the periphery or may be equal or may be unequal or may have combinations thereof. In some embodiments, the root mean square (RMS) value around base power in the sagittal direction may be constant or may vary, for example, RMS=1.0 or RMS<1.0 or RMS, >1.0.

In some embodiments, the m and p components may be optimized for depth of focus and light energy distribution along the optical axis and/or across the retinal image plane by defining the values of the m and/or p components and the slope of the power profiles and/or the shape of the power profiles within a narrow optical zone and/or of an oscillation cycle. For example, an optical zone in the peripheral zone may have a diameter of 2.0 mm and may have a relatively low frequency of 0.5 cycles/mm and defining the m and p components e.g. in a sagittal direction at −5.0 D and +5.0 D, respectively, with a P-V value of 10.0 D therefore the slope of the power change across the cyclical power cycle and between the m and p components may be slow and may form a plurality of light rays over the cycle of higher light energy compared to a higher frequency cycle formed by a narrower optical zone of similar power parameters. In some embodiments, the power profile e.g. at least in a tangential direction, may be provided that further controls the light energy dispersion over a wide range of vergences along the optical axis to form reduced energy focal points in a distribution beneficial for vision correction and/or vision treatment including, for example, by altering “m” and “p” components values and sequence, and/or power progression slopes and/or power progression shapes over a power cycle and/or between “m” and “p” components (e.g., linear, curvilinear or other shape), and/or off axis powers and/or boundary powers.

In some embodiments, independent maximum peak RIQ values and independent Peak RIQ Areas generated at vergences along the through focus RIQ curve may be controlled within the desirable limits using optical principles other than by modifying cyclical power profiles or by using other optical principles in combination with cyclical power profiles in one or more regions across the ophthalmic lens. In some embodiments, the surface geometry or lens matrix may incorporate features that impart lower or higher order aberrations, refraction, diffraction, phase or non-refractive optical principles or any combinations of refractive and/or non-refractive optical principles thereof to modify the independent peak RIQ values and independent peak RIQ areas generated at vergences along the through focus RIQ curve may be controlled within the desirable limits. For example, the lens ID 5 described in FIGS. 6A and 6N-6Q may be redesigned to improve night vision performance by providing a relatively lower light intensity, more evenly distributed across the retinal spot diagram by reducing the maximum peak RIQ value of the independent peak from 0.52 to about 0.45 or lower and to reduce the peak RIQ area to about 0.16 units×Diopters or lower by incorporating, for example, an additional higher order aberration in a portion of the surface geometry on the front and/or back surface of the example lens ID 5. In some embodiments, a non-refractive optical principle such as light scattering features or light amplitude modulating masks may be incorporated over a portion of the center optical zone on one or both surfaces or within at least one or more layers between the lens surfaces in the matrix of the ophthalmic lens.

In some embodiments, the ophthalmic lens may be configured with a central zone located at the center, e.g., the geometrical center or the optical center, of the lens and may be free of narrow optical zones and/or regions of cyclical power profiles. In some embodiments, a portion of the center zone may include, at least in part, narrow optical zones and/or one or more regions of cyclical power profiles that may be used to control the light energy distribution along the optical axis and/or across the retinal image plane within desirable value range limits as disclosed herein. In some embodiments, the center zone may not be located in the center of the lens e.g., the center zone may not be a first optical zone and may be located in a peripheral region and may be positioned inside and/or outside at least a portion of a peripheral zone. In some embodiments, the center zone may be absent e.g. does not exist and its dimension is less than 0.2 mm or less than about 0.1 mm in diameter. In some embodiments, the size of the central zone may alter the light energy intensity along the optical axis and/or the light energy distribution across the retinal image plane to within desirable value range limits as disclosed herein. For example, as the size of the central zone decreases, the peak light energy (e.g., the image quality) may also be reduced. In some soft contact lens or scleral contact lens or intraocular lens embodiments, the dimensions and/or power profiles of the center and peripheral zones including the diameters, widths, curvatures and cyclical power profiles in the sagittal and tangential directions may be configured proportionally to the dimensions and optics of the particular ophthalmic lens device to provide the required power profiles and light energy distribution along the optical axis and across the retinal image plane as disclosed herein. For example, the central zone diameter may be configured proportionally to the overall diameter of the particular ophthalmic lens and also by the position of the lens relative to the anterior surface of the eye. In general, ophthalmic lenses positioned on or in the eye such as a soft contact lens, or hybrid contact lenses or a rigid gas permeable lens or an intraocular lens may have a center zone that may be less than about 9.0 mm and preferably less than 6.0 mm and preferably less than 4.0 mm and more preferably less than 3.0 mm and even more preferably 2.0 mm or less and ideally the central zone may be very small and be 1.0 mm or less. In some embodiments, for example soft contact lenses, or hybrid contact lenses or RGPs or intraocular lenses, the center zone may be about 0.1 mm to 3.0 mm in diameter. In some embodiments, for example a scleral soft contact lenses where lens diameters may be up to 18 or 20 mm, the center zone may be 12 mm or less than 6.0 mm or less than 4.0 mm or less than 3 mm or 2 mm or less. In some embodiments, the central zone may be very small and be 1.0 mm or less. about 0.1 mm to 3.0 mm in diameter. In some embodiments, for example a spectacle lens, the overall lens diameter may be large and up to 40 mm or 50 mm or 70 mm and more and is also fitted in front of the anterior eye surface by a vertex distance of about 10 mm to 18 mm to the spectacle lens and so the central zone may be about 10.0 mm down to about 0.1 mm half chord diameter. In some embodiments, the central zone may have a power profile that may focus light on-axis on and/or in front of and/or behind the retinal image plane. In some embodiments, the center zone may have a power profile that may correct a far distance refractive error and in some other embodiments the central zone may have a power profile that may not have a power profile to correct a far distance refractive error. As disclosed herein the range limits of RIQ peak value and area metrics and CFTEE distributions and slopes of the CFTEE curves may be referenced to a vergence that corresponds to the retinal image plane. In some embodiments, the referenced vergence may correspond to an image plane used for distance or an intermediate or a close-up vision correction in either an accommodating eye or a presbyopic eye with a more limited accommodative range e.g. a low addition, a medium addition or a high addition correction.

In some embodiments, the annular peripheral zone surrounding the center zone may comprise at least one or more narrow annular concentric optical zones. In some embodiments, the narrow optical zones may be formed by lines or curvatures or any geometrical surface shape or any combinations thereof. In some embodiments, the peripheral optical zones e.g., the zones producing the cycles of the cyclical power profiles may be of any size. For example, they may be narrow, for example, 2.0 mm or less, or 1.0 mm or less or very narrow e.g., 0.7 mm or less or 0.5 mm or less or 0.3 mm or less or 0.2 mm or less or 0.1 mm or narrower. In some embodiments, at least a portion of the peripheral zone may incorporate a plurality of narrow optical zones and may have a frequency defined as zones per mm, for example, 1 zone per mm or 1.5 zones per mm or 2 zones per mm or 5 zones per mm or 10 zones per mm or 20 zones per mm or 50 zones per mm or 100 zones per mm or higher frequency.

In some embodiments, the narrow optical zones may be of about equal width or area or may be unequal in width or area or any combinations thereof in order that the light energy may be widely distributed along the optical axis and be of low light intensity and of a light distribution over the retinal image that is of low and even distribution.

In some embodiments, the narrow peripheral optical zones may be, at least in part, annular and concentric and rotationally symmetric, however, in some other embodiments, the zones may also be, at least in part, non-annular, non-concentric and rotationally asymmetric, for example, the zones may form segments or sectors patches or facets and may be of any geometrical shape and/or arranged in any pattern or may be random.

In some embodiments, the zones may be conjoined or may not be conjoined or may be separated by a transition or a blend that may or may not alter the power profile of the narrow peripheral optical zones.

In some embodiments, the zones may form a smooth and continuous surface profile and the tangent angles either side of the zones may be equal or may vary.

In some embodiments, the surface geometry may incorporate features that impart lower or higher order aberrations, refraction, diffraction, phase or non-refractive optical principles or any combinations of refractive and/or non-refractive optical principles thereof.

In some embodiments, for example some of the ophthalmic lenses described in FIG. 6A providing an extended depth of focus useful for vision correction and/or vision treatment and/or providing an acceptable amount of light energy along the optical axis and across the retinal image that may minimize night vision disturbances, may incorporate a plurality of narrow optical zones located in the peripheral region of the ophthalmic lenses that may provide a power profile in at least a tangential direction in the optical zones, for example an off-axis power, that even in combination with the eyeball's optical power of about 45 D to about 55 D, may be high, for example may range from moderately high to very high and may be in the range from about +/−5 D or more or about +/−10 D or more or about or +/−40 D or more or about +/−70 D or more or about +/−100 D or more or about +/−150 D or even higher and may form off-axis focal points inside the eyeball e.g., behind the most anterior surface of the eye and/or on or in front of the retina and/or relatively short distance behind the retina. However, in some embodiments the surface geometry of the plurality of narrow optical zones located in the peripheral region of the ophthalmic lens may be configured so the resultant power profiles, in combination with the eyeball's optical power (e.g., about 45 D to about 55 D), may be low or very low or may be about zero power, for example the net off-axis focal power may be about +/−5 D or less or about +/−3 D or less or about +/−1 D or less or about +/−0.5 D or less and therefore may form off-axis focal points that fall outside the eyeball, for example in the object space in front of the anterior surface of the eyeball as a virtual image and/or on or behind the retinal image plane as a real image.

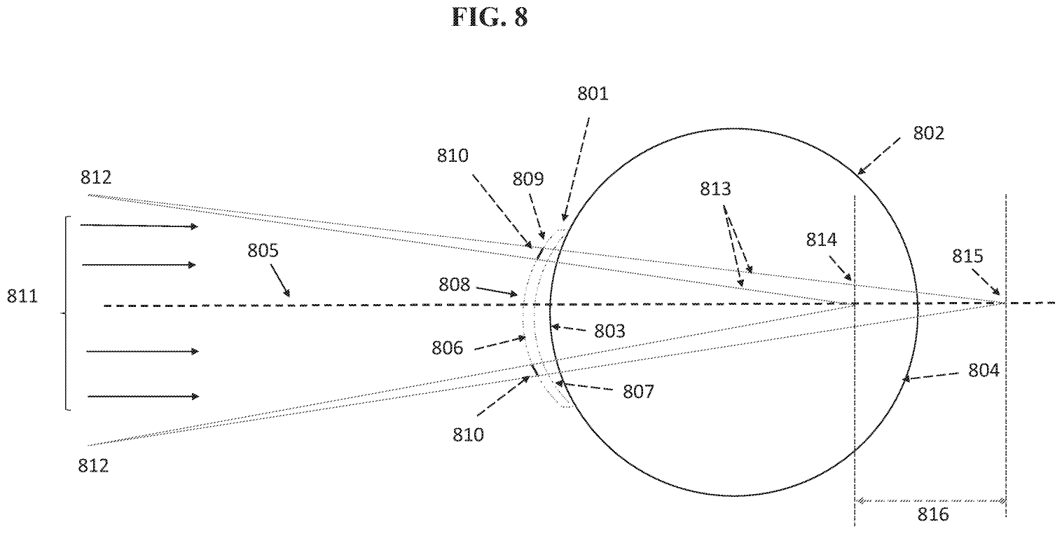

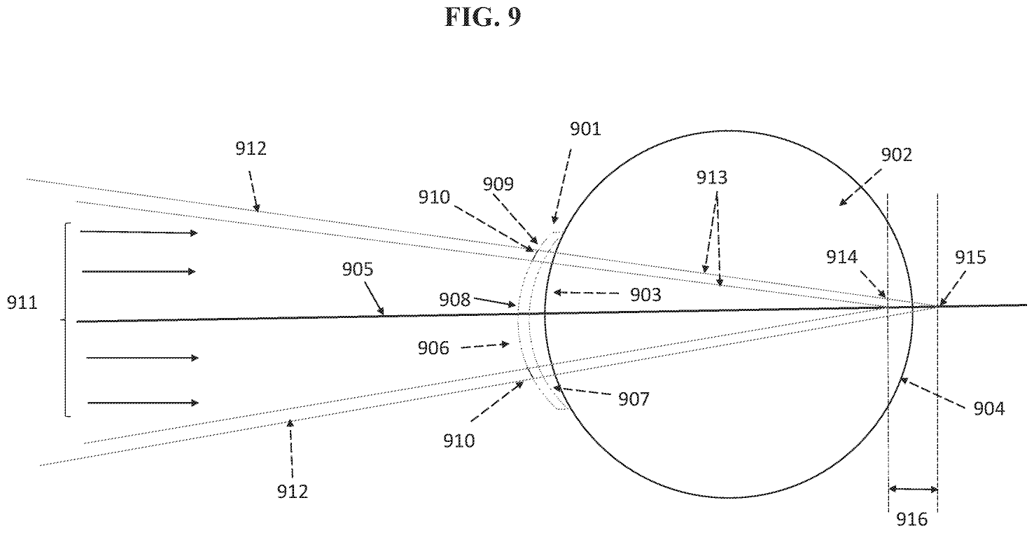

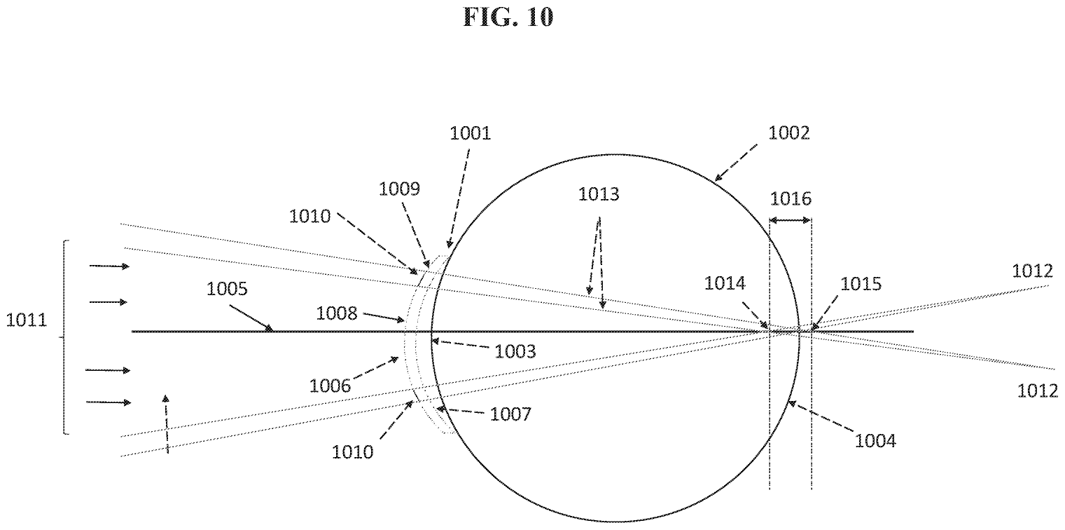

FIGS. 8, 9 and 10 illustrate a cross sectional view of the schematic ray diagrams of select light rays from a far distance object traced through an exemplary ophthalmic lens and anterior eye optical system incorporating an exemplary optical design in accordance with some embodiments described herein incorporating a plurality of narrow optical zones in the peripheral region that may provide, in combination with the optical power of the eyeball, a very low or zero resultant power profile that may form off-axis focal points in the object space in front of the eye (FIG. 8), or may not form off axis focal points (FIG. 9) and/or may form off-axis focal points behind the eyeball (FIG. 10).

The ophthalmic lens illustrated in FIG. 8 is a contact lens 801 and is positioned on the simplified schematic eye 802 and may have an anterior surface e.g., cornea 803 and a posterior surface e.g., retina 804 and may have an optical axis 805. For simplicity of illustration, other optical components and structures of the eyeball such as the corneal curvature, crystalline lens and the anterior and posterior chambers may not be illustrated. The ophthalmic lens (e.g., contact lens) 801 has a front surface 806 and a back surface 807 and a center zone 808 and a peripheral region 809 that may incorporate a plurality of narrow annular, conjoined optical zones (for illustrative purposes only one of the annular optical zones 810 on the front surface 806 is drawn in cross section). The narrow optical zone 810 may be configured with a line curvature and may form a cyclical power profile that may provide an off-axis power profile of about −54 D in the object space but when combined with the optical power of the eyeball 802 of +50 D may result in a small net resultant power profile of about −4 D. Consequently, parallel light rays 811 originating from a distant object may form a virtual image 812 well in front of the anterior surface of the eyeball 802 and contact lens 801. The light rays 813 diverge from the focal point 812 formed by the contact lens-eyeball optical system towards the retinal image plane 804 and intersect at the optical axis 805 and form on-axis focal points 814 and 815 of reduced energy level and the distance between the 2 focal points 816 may indicate the length over which the light energy is dispersed along the optical axis. The collection of on-axis focal points formed along the optical axis from light rays from the off-axis virtual image from the very low power profile of the resulting optical system of the eyeball 802 and the plurality of narrow optical zones e.g., 810 in the peripheral region 809 may form at least one or more peak RIQ values and peak RIQ areas on the through focus RIQ curve and a light energy distribution across the retinal image plane within the predetermined acceptable limits that may provide an extended depth of focus useful for vision correction and/or vision treatment and/or also mitigate, reduce and/or prevent night visual disturbances such as glare, haloes and/or starbursts.

The ophthalmic lens illustrated in FIG. 9 is a contact lens 901 and is positioned on the simplified schematic eye 902 and may have an anterior surface e.g., cornea 903 and a posterior surface e.g., retina 904 and may have an optical axis 905. For simplicity of illustration, other optical components, and structures of the eyeball such as the corneal curvature, crystalline lens and the anterior and posterior chambers may not be illustrated. The contact lens 901 has a front surface 906 and a back surface 907 and a center zone 908 and a peripheral region 909 that may incorporate a plurality of narrow annular, conjoined optical zones (for illustrative purposes only one of the annular optical zones 910 on the front surface 906 is drawn in cross section). The narrow optical zone 910 may be configured with a line curvature and may form a cyclical power profile that may provide an off-axis power profile of about −50 D in the object space but when combined with the optical power of the eyeball 902 of +50 D may result in a net resultant power profile of about 0 D. Consequently, parallel light rays 911 originating from a distant object may remain parallel and may not form an off-axis focal point either in front of or behind the anterior surface of the eyeball 902 and contact lens 901 or on the retinal image plane 904. The parallel light rays 911 continue their parallel path through the contact lens—eyeball optical system and intersect the optical axis 905 to form on-axis focal points 914 and 915 either side of the retinal image plane 904 and the distance between the 2 on axis focal points 916 may indicate the extent of light energy dispersion along the optical axis. The collection of reduced energy focal points dispersed widely along the optical axis by the parallel light from the about zero power profile resulting from the plurality of narrow optical zones e.g., 910 in the peripheral region 909, and the optical system of the eyeball 902, may, without forming off axis focal points, provide at least one or more peak RIQ values and RIQ areas on the through focus RIQ curve and a light energy distribution across the retinal image plane, within the predetermined acceptable limits that may provide an extended depth of focus useful for vision correction and/or vision treatment and/or also mitigate, reduce and/or prevent night visual disturbances such as glare, haloes and/or starbursts.

The ophthalmic lens illustrated in FIG. 10 is a contact lens 1001 and is positioned on the simplified schematic eye 1002 and may have an anterior surface e.g., cornea 1003 and a posterior surface e.g., retina 1004 and may have an optical axis 1005. For simplicity of illustration, other optical components, and structures of the eyeball such as the corneal curvature, crystalline lens and the anterior and posterior chambers may not be illustrated. The contact lens 1001 has a front surface 1006 and a back surface 1007 and a center zone 1008 and a peripheral region 1009 that may incorporate a plurality of narrow annular, conjoined optical zones (for illustrative purposes only one of the annular optical zones 1010 on the front surface 1006 is drawn in cross section). The narrow optical zone 1010 may be configured with a line curvature and may form a cyclical power profile that may provide an off-axis power profile of about −45 D in the object space but when combined with the optical power of the eyeball 1002 of +50 D may result in a small net resultant power profile of about +5 D. Consequently, parallel light rays 1011 originating from a distant object may form a real image 1012 off axis well behind the posterior surface of the eyeball 1004 and contact lens 1001. The light rays 1013 converge toward the focal point 1012 formed by the contact lens—eyeball optical system behind the retinal image plane 1004 and intersect at the optical axis 1005 and form on-axis focal points 1014 and 1015 and the distance between the two on axis focal points 1016 may indicate the extent of light energy dispersion along the optical axis. The collection of reduced energy focal points dispersed widely along the optical axis by the power profile of the resulting optical system of the eyeball 1002 and the plurality of narrow optical zones e.g. 1010 in the peripheral region 1009, may form at least one or more peak RIQ values and peak RIQ areas on the through focus RIQ curve and a light energy distribution across the retinal image plane within the predetermined acceptable limits that may provide an extended depth of focus useful for vision correction and/or vision treatment and/or also mitigate, reduce and/or prevent night visual disturbances such as glare, haloes and/or starbursts.

Further advantages of the claimed subject matter will become apparent from the following examples describing certain embodiments of the claimed subject matter. In certain embodiments, one or more than one (including for instance all) of the following further embodiments may comprise each of the other embodiments or parts thereof.

### EXAMPLES

### A Examples

A1. An ophthalmic lens configured to correct and/or treat at least one condition of the eye (e.g., presbyopia, myopia, hyperopia, astigmatism, binocular vision disorders and/or visual fatigue syndrome) comprising: a central optical zone; a peripheral optical zone; a base power profile; and at least one feature selected to modify the base power profile and to form one or more off-axis focal points in front of, on, and/or behind a retinal image plane and reduce a focal point energy level at one or more image planes; wherein the at least one feature may be located on a front surface and/or a back surface of at least one of the central optical zone and the peripheral optical zone.

A2. The ophthalmic lens of any of the A examples, wherein the at least one feature comprises at least one narrow optical zone incorporating one or more cyclical power profiles and forming one or more off-axis focal points and one or more on-axis focal points along the optical axis.

A3. The ophthalmic lens of any of the A examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3.1 D, ±3.2 D, and/or ±3.25 D)), and wherein (1) the maximum RIQ value of the independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks may be less than about 0.45 (e.g., 0.42, 0.43, 0.44, 0.45, 0.46, 0.47 or 0.48).

A4. The ophthalmic lens of any of the A examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D±3D, ±3.1 D±3.2 D, and/or ±3.25 D)), and wherein an RIQ area of the one or more independent peaks may be about 0.16 Units*Diopters (e.g., 0.13, 0.14, 0.15, 0.16, 0.17, 0.18 or 0.19) or less.

A5. The ophthalmic lens of any of the A examples, wherein the ophthalmic lens provides a through focus retinal image quality (RIQ) with one or more (e.g., 1, 2, 3, 4, or 5) independent peaks (e.g., over a vergence range of about ±3.0 D (e.g., ±2.75 D, ±2.8 D, ±2.9 D, ±3D, ±3.1 D, ±3.2 D, and/or ±3.25 D)), and wherein there may be at least one or more independent peaks (e.g., 1, 2, 3, 4, or 5 peaks).

A6. The ophthalmic lens of any of the A examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens.

A7. The ophthalmic lens of any of the A examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein a peak-to-valley (P-to-V) power range between the absolute powers of the “m” and “p” components of the cycle of the cyclical power profile in the sagittal direction may be about 200 D, about 150 D, about 100 D, about 75 D, about 50 D, about 40 D, about 30 D, about 20 D, about 10 D, about 5 D or less, about 4 D less, about 3D or less, and/or about 2D or less.

A8. The ophthalmic lens of any of the A examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the peak-to-valley (P-to-V) power range between the absolute powers of the “m” and “p” components of the cycle of the cyclical power profile in the tangential direction may be about 600 D, about 500 D, about 400 D, about 300 D, about 200 D, about 175 D, about 150 D, about 125 D, about 100 D, about 75 D, about 60 D, about 50 D, about 40 D, about 35 D, and/or about 30 D or less.

A9. The ophthalmic lens of any of the A examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the frequency of the cyclical power profile may be about 0.5, 1, 1.5, 2, 3, 4, 5, 6, 7, 8, 9, 10, 15, 20, 50 and/or 100 cycles/mm.

A10. The ophthalmic lens of any of the A examples, wherein the at least one feature comprises a line curvature (e.g., a cyclical power profile formed by a line curvature).

A11. The ophthalmic lens of any of the A examples, wherein the at least one feature comprises a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones.

A12. The ophthalmic lens of any of the A examples, wherein the at least one feature comprises a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones that may be between about 20-2000 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, about 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 775 μm, 800 μm, 825 μm, 850 μm, 875 μm, 900 μm, 925 μm, 950 μm, 975 μm, 1000 μm, 1025 μm, 1050 μm, 1075 μm, 1100 μm, 1125 μm, 1150 μm, 1175 μm, 1200 μm, 1225 μm, 1250 μm, 1275 μm, 1300 μm, 1325 μm, 1350 μm, 1375 μm, 1400 μm, 1525 μm, 1550 μm, 1575 μm, 1600 μm, 1625 μm, 1650 μm, 1675 μm, 1700 μm, 1725 μm, 1750 μm, 1775 μm, 1800 μm, 1825 μm, 1850 μm, 1875 μm, 1900 μm, 1925 μm, 1950 μm, 1975 μm, 2000 μm, 2025 μm, 2050 μm, 2075 μm, and/or 2100 μm wide).

A13. The ophthalmic lens of any of the A examples, wherein the at least one feature comprises a plurality of narrow and/or annular concentric optical zones located on at least one of the front surface and/or the back surface of the ophthalmic lens and formed by line curvatures.

A14. The ophthalmic lens of any of the A examples, wherein the at least one feature comprises a plurality of narrow and/or annular concentric optical zones and a net resultant power profile of the narrow and/or annular zones of the peripheral zone may be at least one of relatively more positive in power than the central zone, relatively more negative in power than the central zone, and/or about the same power as the central zone.

A15. The ophthalmic lens of any of the A examples, wherein the at least one feature comprises a plurality of narrow and/or annular concentric optical zones and the plurality of narrow and/or annular concentric zones may be conjoined (e.g., the spacing between the two adjacent optical zones may be substantially zero and the innermost and the outermost portion of the surface curvature of the narrow and/or annular concentric zones transition to the base curve) with an adjacent narrow and/or annular concentric optical zone.

A16. The ophthalmic lens of any of the A examples, wherein the at least one feature comprises a plurality of narrow and/or annular concentric optical zones and the plurality of narrow and/or annular concentric zones may be spaced apart from one another so as to create an alternating pattern where the base power profile (or a power other than the base power) alternates with the narrow and/or annular concentric zones.

A17. The ophthalmic lens of any of the A examples, wherein the at least one feature comprises a plurality of narrow and/or annular concentric optical zones and the plurality of narrow and/or annular concentric zones may be configured so that the innermost and outermost portions of at least one of the narrow and/or annular concentric optical zones may be geometrically normal to the surface and provides a lateral separation of the focal points (e.g., infinite number of focal points) formed by the narrow and/or annular concentric optical zones from the optical axis.

A18. The ophthalmic lens of any of the A examples, wherein the at least one feature comprises a plurality of narrow and/or annular concentric optical zones and the light energy and/or image quality formed by the plurality of narrow and/or annular concentric optical zones may be substantially similar and/or dissimilar.

A19. The ophthalmic lens of any of the A examples, wherein the at least one feature comprises a plurality of narrow and/or annular concentric optical zones and one of the plurality of narrow and/or annular concentric optical zones form a single cycle of oscillation of power (e.g., one or both of sagittal and tangential) around the base power profile (e.g., the base power profile of the central optical zone).

A20. The ophthalmic lens of any of the A examples, wherein the at least one feature comprises a plurality of narrow and/or annular concentric optical zones and the power range between the absolute powers of “p” and “m” components in the single power profile cycle (e.g., the peak to valley or P-to-V value) may be at least one of constant or varying (e.g., increasing, decreasing, and or randomly changing) in at least one direction across the optical zone.

A21. The ophthalmic lens of any of the A examples, wherein a combination of at least one or more of the central optical zone size, the plurality of narrow and/or annular concentric optical zones, the front surface curvature, lens thickness, back surface curvature, and the refractive index may be configured to form a power profile across the central and peripheral optical zones such that the ophthalmic lens forms on-axis focal points and off-axis focal points over a substantially wide range of vergences to provide an appropriate range of light energy distributions along the optical axis and across the retinal image plane that correct/treat the refractive condition of the eye by extending the depth of focus along the optical axis at least in part on and/or in front of the retina of the eye to extend the depth of focus and/or to reduce, mitigate or prevent one or more night vision disturbances that accompany the use of such ophthalmic devices.

A22. The ophthalmic lens of any of the A examples, wherein the at least one feature comprises a plurality of narrow and/or annular concentric optical zones and wherein light rays from the plurality of narrow and/or annular concentric optical zones provide a low light energy.

A23. The ophthalmic lens of any of the A examples, wherein an interference from light rays created by the plurality of narrow and/or annular concentric optical zones increases and/or decreases from the anterior most image plane from retina to the posterior most (e.g., retinal) image plane or decreases from the retinal image plane (or another image plane) to at least one of the anterior most image plane and the posterior most image plane.

A24. The ophthalmic lens of any of the A examples, wherein any combination of at least one or more of the central optical zone diameter and/or the power profile of at least a portion of the ophthalmic lens may be used to provide a desirable condition to reduce/minimize light interference on in-focus images by out-of-focus images and/or to reduce, mitigate, or prevent one or more night vision disturbances (e.g. by adjusting one or more of on-axis and/or off-axis focal point and image plane location, light energy, image quality, total enclosed energy distribution, and/or depth of focus).

A25. The ophthalmic lens of any of the A examples, wherein, any combination of one or more of the number of narrow and/or annular concentric optical zones and/or width and/or sagittal power profile and/or tangential power profile and/or boundary power profile and/or m:p ratio (e.g., RMS) and/or P-to-V value and/or surface curvature and/or lateral separation and/or spacing and/or surface location of the optical zones may be used to provide a desirable condition to extend depth of focus, to reduce focal point energy levels, to reduce/minimize light interference on in-focus images by out-of-focus images and/or to reduce, mitigate, or prevent one or more night vision disturbances (e.g., by adjusting one or more of on-axis and/or off-axis focal point and image plane location, light energy, image quality, total enclosed energy distribution, and/or depth of focus).

A26. The ophthalmic lens of any of the A examples, wherein the ophthalmic lens provides, at least in part, an extended depth of focus within the useable vergence ranges encountered by a user of the ophthalmic lens.

A27. The ophthalmic lens of any of the A examples, wherein the one or more on-axis focal points has a low light energy along the optical axis of the ophthalmic lens.

A28. The ophthalmic lens of any of the A examples, wherein the ophthalmic lens is configured to provide a low light energy formed on the retina.

A29. The ophthalmic lens of any of the A examples, wherein light rays that form one or more off-axis focal points may be distributed across a substantially wide range of vergences along the optical axis and in front of, on, and/or behind the retinal image plane of an eye in use.

A30. The ophthalmic lens of any of the A examples, wherein the ophthalmic lens has a uniform or relatively uniform light ray intensity distribution across the retinal spot diagram.

A31. The ophthalmic lens of any of the A examples, wherein a total enclosed energy that results at the retinal image plane may be determined from a retinal spot diagram, and at least more than about 50% (e.g., 45%, 50%, and/or 55%) of the total enclosed energy may be distributed beyond a 35 μm, 40 μm, 45 μm, 50 μm, 55 μm, 60 μm, 65 μm, 70 μm, 75 μm, 80 μm, and/or 95 μm half chord diameter of the retinal spot diagram.

A32. The ophthalmic lens of any of the A examples, wherein a cumulative fraction of a total enclosed energy that results at the retinal image plane has an average slope of less than about 0.13 units/10 μm (e.g., about 0.11 units/10 μm, 0.12 units/10 μm, 0.125 units/10 μm, 0.13 units/10 μm, 0.14 units/10 μm, and/or 0.15 units/10 μm or less) over 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

A33. The ophthalmic lens of any of the A examples, wherein the central optical zone has a half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, about 0.1 mm or less.

A34. The ophthalmic lens of any of the A examples, wherein the at least one feature may be configured to reduce, mitigate and/or prevent one or more night vision disturbances (e.g., any combination of one or more of glare, haloes and/or starbursts).

A35. The ophthalmic lens of any of the A examples, wherein the ophthalmic lens may be one of a contact lens, an intraocular lens, and/or a spectacle lens.

### B Examples

B1. An ophthalmic lens configured to correct and/or treat at least one condition of the eye (e.g., presbyopia, myopia, hyperopia, astigmatism, binocular vision disorders and/or visual fatigue syndrome) comprising: an optical zone; a base power profile; and at least one feature selected to modify the base power profile and to form one or more off-axis focal points in front of, on, and/or behind a retinal image plane and reduce a focal point energy level at one or more image planes; wherein the at least one feature may be located on a front surface and/or a back surface of the optical zone.

B2. The ophthalmic lens of any of the B examples, wherein the at least one feature comprises at least one narrow optical zone incorporating one or more cyclical power profiles and forming one or more off-axis focal points and one or more on-axis focal points along the optical axis.

B3. The ophthalmic lens of any of the B examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3.1 D, ±3.2 D, and/or ±3.25 D)), and wherein (1) the maximum RIQ value of the independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks may be less than about 0.45 (e.g., 0.42, 0.43, 0.44, 0.45, 0.46, 0.47 or 0.48).

B4. The ophthalmic lens of any of the B examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3.1 D, ±3.2 D, and/or ±3.25 D)), and wherein an RIQ area of the one or more independent peaks may be about 0.16 Units*Diopters (e.g., 0.13, 0.14, 0.15, 0.16, 0.17, 0.18 or 0.19) or less.

B5. The ophthalmic lens of any of the B examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3.1 D, ±3.2 D, and/or ±3.25 D)), and wherein there may be at least one independent peak (e.g., 1, 2, 3, 4, or 5 peaks).

B6. The ophthalmic lens of any of the B examples, wherein the ophthalmic lens (e.g., the at least one feature of the ophthalmic lens) comprises a cyclical power profile comprising one or more cycles across a portion of the ophthalmic lens and the cycle of the cyclical power profile incorporates a “m” component that may be relatively more negative in power than the base power profile of the ophthalmic lens and a “p” component that may be relatively more positive than the base power profile of the ophthalmic lens; and wherein the frequency of the cyclical power profile may be about 0.5, 1, 1.5, 2, 3, 4, 5, 6, 7, 8, 9, 10, 15, 20, 50, and/or 100 cycles/mm.

B7. The ophthalmic lens of any of the B examples, wherein the at least one feature comprises a plurality of narrow and/or annular concentric optical zones and the power range between the absolute powers of “p” and “m” components in the single power profile cycle (e.g., the peak to valley or P-to-V value) may be at least one of constant or varying (e.g., increasing, decreasing, and or randomly changing) in at least one direction across the optical zone.

B8. The ophthalmic lens of any of the B examples, wherein, any combination of one or more of the number of narrow and/or annular concentric optical zones and/or width and/or sagittal power profile and/or tangential power profile and/or boundary power profile and/or m:p ratio (e.g., RMS) and/or P-to-V value and/or surface curvature and/or lateral separation and/or spacing and/or surface location of the optical zones may be used to provide a desirable condition to extend depth of focus, to reduce focal point energy levels, to reduce/minimize light interference on in-focus images by out-of-focus images and/or to reduce, or mitigate, or prevent one or more night vision disturbances (e.g., by adjusting one or more of on-axis and/or off-axis focal point and image plane location, light energy, image quality, total enclosed energy distribution, and/or depth of focus).

B9. The ophthalmic lens of any of the B examples, wherein the ophthalmic lens (e.g., the at least one feature of the ophthalmic lens) comprises a cyclical power profile comprising one or more cycles across a portion of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens.

B10. The ophthalmic lens of any of the B examples, wherein the ophthalmic lens (e.g., the at least one feature of the ophthalmic lens) comprises a cyclical power profile comprising one or more cycles across a portion of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and

wherein a peak-to-valley (P-to-V) power range between the absolute powers of the “m” and “p” components of the cycle of the cyclical power profile in the sagittal direction may be about 200 D, about 150 D, about 100 D, about 75 D, about 50 D, about 40 D, about 30 D, about 20 D, about 10 D, about 5 D or less, about 4 D or less, about 3D or less, and/or about 2D or less.

B11. The ophthalmic lens of any of the B examples, wherein the ophthalmic lens (e.g., the at least one feature of the ophthalmic lens) comprises a cyclical power profile comprising one or more cycles across a portion of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the peak-to-valley (P-to-V) power range between the absolute powers of the “m” and “p” components of the cycle of the cyclical power profile in the tangential direction may be about 600 D, about 500 D, about 400 D, about 300 D, about 200 D, about 175 D, about 150 D, about 125 D, about 100 D, about 75 D, about 60 D, about 50 D, about 40 D, about 35 D, and/or about 30 D or less.

B12. The ophthalmic lens of any of the B examples, wherein the at least one feature comprises a line curvature (e.g., a cyclical power profile formed by a line curvature).

B13. The ophthalmic lens of any of the B examples, wherein the at least one feature comprises a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones.

B14. The ophthalmic lens of any of the B examples, wherein the at least one feature comprises a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones that may be between about 20-2000 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, about 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 775 μm, 800 μm, 825 μm, 850 μm, 875 μm, 900 μm, 925 μm, 950 μm, 975 μm, 1000 μm, 1025 μm, 1050 μm, 1075 μm, 1100 μm, 1125 μm, 1150 μm, 1175 μm, 1200 μm, 1225 μm, 1250 μm, 1275 μm, 1300 μm, 1325 μm, 1350 μm, 1375 μm, 1400 μm, 1525 μm, 1550 μm, 1575 μm, 1600 μm, 1625 μm, 1650 μm, 1675 μm, 1700 μm, 1725 μm, 1750 μm, 1775 μm, 1800 μm, 1825 μm, 1850 μm, 1875 μm, 1900 μm, 1925 μm, 1950 μm, 1975 μm, 2000 μm, 2025 μm, 2050 μm, 2075 μm, and/or 2100 μm wide).

B15. The ophthalmic lens of any of the B examples, wherein the at least one feature comprises a plurality of narrow and/or annular concentric optical zones located on at least one of the front surface and/or the back surface of the ophthalmic lens and formed by line curvatures.

B16. The ophthalmic lens of any of the B examples, wherein the at least one feature comprises a plurality of narrow and/or annular concentric optical zones and a net resultant power profile of the narrow and/or annular zones may be at least one of relatively more positive in power than the base power profile, relatively more negative in power than the central zone, and/or about the same power as the central zone.

B17. The ophthalmic lens of any of the B examples, wherein the at least one feature comprises a plurality of narrow and/or annular concentric optical zones and the plurality of narrow and/or annular concentric optical zones may be conjoined (e.g., the spacing between the two adjacent narrow and/or annular concentric optical zones may be substantially zero and the innermost and the outermost portion of the surface curvature of the narrow and/or annular concentric zones transition to the base curve) with an adjacent narrow and/or annular concentric optical zone.

B18. The ophthalmic lens of any of the B examples, wherein the at least one feature comprises a plurality of narrow and/or annular concentric optical zones and the plurality of narrow and/or annular concentric zones may be spaced apart from one another so as to create an alternating pattern where the base power profile (or a power other than the base power) alternates with the narrow and/or annular concentric zones.

B19. The ophthalmic lens of any of the B examples, wherein the at least one feature comprises a plurality of narrow and/or annular concentric optical zones and the plurality of narrow and/or annular concentric zones may be configured so that the innermost and outermost portions of at least one of the narrow and/or annular concentric optical zones may be geometrically normal to the surface and provides a lateral separation of the focal points (e.g., infinite number of focal points) formed by the narrow and/or annular concentric optical zones from the optical axis.

B20. The ophthalmic lens of any of the B examples, wherein the at least one feature comprises a plurality of narrow and/or annular concentric optical zones and the light energy and/or image quality formed by the plurality of narrow and/or annular concentric optical zones may be substantially similar and/or dissimilar.

B21. The ophthalmic lens of any of the B examples, wherein the at least one feature comprises a plurality of narrow and/or annular concentric optical zones and one of the plurality of narrow and/or annular concentric optical zones form a single cycle of oscillation of power (e.g., one or both of sagittal and tangential) around the base power profile.

B22. The ophthalmic lens of any of the B examples, wherein a combination of at least one or more of the plurality of narrow and/or annular concentric optical zones, the front surface curvature, lens thickness, back surface curvature, and the refractive index may be configured to form a power profile across the optical zone such that the ophthalmic lens forms on-axis focal points and off-axis focal points over a substantially wide range of vergences to provide an appropriate range of light energy distributions along the optical axis and across the retinal image plane that correct/treat the refractive condition of the eye by extending the depth of focus along the optical axis at least in part on and/or in front of the retina of the eye and reduce the light intensity at a retinal plane during use to extend the depth of focus and/or to reduce, mitigate or prevent one or more night vision disturbances that accompany the use of such ophthalmic devices.

B23. The ophthalmic lens of any of the B examples, wherein the at least one feature comprises a plurality of narrow and/or annular concentric optical zones and wherein light rays from the plurality of narrow and/or annular concentric optical zones provide a low light energy.

B24. The ophthalmic lens of any of the B examples, wherein an interference from light rays created by the plurality of narrow and/or annular concentric optical zones increases and/or decreases from the anterior most image plane from retina to the posterior most (e.g., retinal) image plane or decreases from the retinal image plane (or another image plane) to at least one of the anterior most image plane and the posterior most image plane.

B25. The ophthalmic lens of any of the B examples, wherein any combination of at least one or more of the central optical zone diameter and/or the power profile of at least a portion of the ophthalmic lens may be used to provide a desirable condition to reduce/minimize light interference on in-focus images by out-of-focus images and/or to reduce, or mitigate, or prevent one or more night vision disturbances (e.g. by adjusting one or more of on-axis and/or off-axis focal point and image plane location, light energy, image quality, total enclosed energy distribution, and/or depth of focus).

B26. The ophthalmic lens of any of the B examples, wherein the ophthalmic lens provides, at least in part, an extended depth of focus within the useable vergence ranges encountered by a user of the ophthalmic lens.

B27. The ophthalmic lens of any of the B examples, wherein the one or more on-axis focal points has a low light energy along the optical axis of the ophthalmic lens.

B28. The ophthalmic lens of any of the B examples, wherein the ophthalmic lens is configured to provide a low light energy formed on the retina.

B29. The ophthalmic lens of any of the B examples, wherein light rays that form one or more off-axis focal points may be distributed across a substantially wide range of vergences along the optical axis and in front of, on and/or behind the retinal image plane of an eye in use.

B30. The ophthalmic lens of any of the B examples, wherein the ophthalmic lens has a uniform or relatively uniform light intensity distribution across the retinal spot diagram.

B31. The ophthalmic lens of any of the B examples, wherein a total enclosed energy that results at the retinal image plane may be determined from a retinal spot diagram, and at least more than about 50% (e.g., 45%, 50%, and/or 55%) of the total enclosed energy may be distributed beyond a 35 μm, 40 μm, 45 μm, 50 μm, 55 μm, 60 μm, 65 μm, 70 μm, 75 μm, 80 μm, and/or 95 μm half chord diameter of the retinal spot diagram.

B32. The ophthalmic lens of any of the B examples, wherein a cumulative fraction of a total enclosed energy that results at the retinal image plane has an average slope of less than about 0.13 units/10 μm (e.g., about 0.11 units/10 μm, 0.12 units/10 μm, 0.125 units/10 μm, 0.13 units/10 μm, 0.14 units/10 μm, and/or 0.15 units/10 μm or less) over 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

B33. The ophthalmic lens of any of the B examples, wherein the ophthalmic lens comprises a central zone and the central optical zone has a half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, about 0.1 mm or less.

B34. The ophthalmic lens of any of the B examples, wherein the at least one feature may be configured to reduce, mitigate and/or prevent one or more night vision disturbances (e.g., any combination of one or more of glare, haloes and/or starbursts).

B35. The ophthalmic lens of any of the B examples, wherein the ophthalmic lens may be one of a contact lens, an intraocular lens, and/or a spectacle lens.

### C Examples

C1. An ophthalmic lens comprising: a front surface; a back surface; a central optical zone; an annular peripheral optical zone surrounding the central optical zone; and an optical design formed on at least one of the front surface or the back surface of the ophthalmic lens; wherein the optical design comprises a power profile (e.g., a cyclical or non-cyclical power profile) in the central optical zone that forms at least one focal point along an optical axis (e.g., in front of, on and/or behind the retinal image plane); and wherein the optical design comprises a power profile in the annular peripheral optical zone comprising at least one or more narrow and/or annular conjoined optical zones that have a cyclical power profile and form one or more off-axis focal points (e.g., in front of, on, and/or behind the retinal image plane)

C2. The ophthalmic lens of any of the C examples, wherein the at least one or more narrow and/or annular conjoined optical zones form one or more on-axis focal points along the optical axis (e.g., in front of, on and/or behind the retinal image plane and/or in front of, on and/or behind the on-axis focal point formed by the central optical zone).

C3. The ophthalmic lens of any of the C examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3.1 D, ±3.2 D, and/or ±3.25 D)), and wherein (1) the maximum RIQ value of the independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks may be less than about 0.45 (e.g., 0.42, 0.43, 0.44, 0.45, 0.46, 0.47 or 0.48).

C4. The ophthalmic lens of any of the C examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3.1 D, ±3.2 D, and/or ±3.25 D)), and wherein an RIQ area of the one or more independent peaks may be about 0.16 Units*Diopters (e.g., 0.13, 0.14, 0.15, 0.16, 0.17, 0.18 or 0.19) or less.

C5. The ophthalmic lens of any of the C examples, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3.1 D, ±3.2 D, and/or ±3.25 D)), and wherein there may be at least one independent peak (e.g., 1, 2, 3, 4, or 5 peaks) peaks.

C6. The ophthalmic lens of any of the C examples, wherein the power profile in the annular peripheral optical zone comprises a plurality of narrow and/or annular conjoined optical zones the power range between the absolute powers of “p” and “m” components in the single power profile cycle (e.g., the peak to valley or P-to-V value) may be at least one of constant or varying (e.g., increasing, decreasing, and or randomly changing) in at least one direction across the optical zone.

C7. The ophthalmic lens of any of the C examples, wherein, any combination of one or more of the number of narrow and/or annular conjoined optical zones and/or width and/or sagittal power profile and/or tangential power profile and/or boundary power profile and/or m:p ratio (e.g., RMS) and/or P-to-V value and/or surface curvature and/or lateral separation and/or spacing and/or surface location of the optical zones may be used to provide a desirable condition to extend depth of focus, to reduce focal point energy levels, to reduce/minimize light interference on in-focus images by out-of-focus images and/or to reduce, mitigate, or prevent one or more night vision disturbances (e.g., by adjusting one or more of on-axis and/or off-axis focal point and image plane location, light energy, image quality, total enclosed energy distribution, and/or depth of focus).

C8. The ophthalmic lens of any of the C examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens.

C9. The ophthalmic lens of any of the C examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein a peak-to-valley (P-to-V) power range between the absolute powers of the “m” and “p” components of the cycle of the cyclical on-axis power profile in the sagittal direction may be about 200 D, about 150 D, about 100 D, about 75 D, about 50 D, about 40 D, about 30 D, about 20 D, about 10 D, about 5 D or less, about 4 D or less, about 3D or less and/or about 2D or.

C10. The ophthalmic lens of any of the C examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the peak-to-valley (P-to-V) power range between the absolute powers of the “m” and “p” components of the cycle of the cyclical power profile in the tangential direction may be about 600 D, about 500 D, about 400 D, about 300 D, about 200 D, about 175 D, about 150 D, about 125 D, about 100 D, about 75 D, about 60 D, about 50 D, about 40 D, about 35 D, and/or about 30 D or less.

C11. The ophthalmic lens of any of the C examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the frequency of the cyclical power profile may be about 0.5, 1, 1.5, 2, 3, 4, 5, 6, 7, 8, 9, 10, 15, 20, 50, and/or 100 cycles/mm.

C12. The ophthalmic lens of any of the C examples, wherein the power profile in the annular peripheral optical zone comprises a line curvature (e.g., a cyclical power profile formed by a line curvature).

C13. The ophthalmic lens of any of the C examples, wherein the power profile in the annular peripheral optical zone comprises a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular conjoined optical zones.

C14. The ophthalmic lens of any of the C examples, wherein the power profile in the annular peripheral optical zone comprises a plurality of narrow and/or annular conjoined optical zones that may be between about 20-2000 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, about 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 775 μm, 800 μm, 825 μm, 850 μm, 875 μm, 900 μm, 925 μm, 950 μm, 975 μm, 1000 μm, 1025 μm, 1050 μm, 1075 μm, 1100 μm, 1125 μm, 1150 μm, 1175 μm, 1200 μm, 1225 μm, 1250 μm, 1275 μm, 1300 μm, 1325 μm, 1350 μm, 1375 μm, 1400 μm, 1525 μm, 1550 μm, 1575 μm, 1600 μm, 1625 μm, 1650 μm, 1675 μm, 1700 μm, 1725 μm, 1750 μm, 1775 μm, 1800 μm, 1825 μm, 1850 μm, 1875 μm, 1900 μm, 1925 μm, 1950 μm, 1975 μm, 2000 μm, 2025 μm, 2050 μm, 2075 μm, and/or 2100 μm wide).

C15. The ophthalmic lens of any of the C examples, wherein the power profile in the annular peripheral optical zone comprises a plurality of narrow and/or annular conjoined optical zones located on at least one of the front surface and/or the back surface of the ophthalmic lens and formed by line curvatures.

C16. The ophthalmic lens of any of the C examples, wherein the power profile in the annular peripheral optical zone comprises a plurality of narrow and/or annular conjoined optical zones and a net resultant power profile of the narrow and/or annular conjoined optical zones of the annular peripheral optical zone may be at least one of relatively more positive in power than the central optical zone, relatively more negative in power than the central zone, and/or about the same power as the central zone.

C17. The ophthalmic lens of any of the C examples, wherein the power profile in the annular peripheral optical zone comprises a plurality of narrow and/or annular conjoined optical zones and the plurality of narrow and/or annular conjoined optical zones may be conjoined (e.g., the spacing between the two adjacent optical zones may be substantially zero and the innermost and the outermost portion of the surface curvature of the narrow and/or annular conjoined optical zones transition to the base curve) with an adjacent narrow and/or annular conjoined optical zones.

C18. The ophthalmic lens of any of the C examples, wherein the power profile in the annular peripheral optical zone comprises a plurality of narrow and/or annular conjoined optical zones and the plurality of narrow and/or annular conjoined optical zones may be spaced apart from one another so as to create an alternating pattern where the spacing between the two adjacent narrow and/or annular conjoined optical zones may be non-zero.

C19. The ophthalmic lens of any of the C examples, wherein the power profile in the annular peripheral optical zone comprises a plurality of narrow and/or annular conjoined optical zones and the plurality of narrow and/or annular conjoined optical zones may be configured so that the innermost and outermost portions of at least one of the narrow and/or annular conjoined optical zones may be geometrically normal to the surface and provides a lateral separation of the focal points (e.g., infinite number of focal points) formed by the narrow and/or annular conjoined optical zones from the optical axis.

C20. The ophthalmic lens of any of the C examples, wherein the power profile in the annular peripheral optical zone comprises a plurality of narrow and/or annular conjoined optical zones and the light energy and/or image quality formed by the plurality of narrow and/or annular conjoined optical zones may be substantially similar and/or dissimilar.

C21. The ophthalmic lens of any of the C examples, wherein the power profile in the annular peripheral optical zone comprises a plurality of narrow and/or annular conjoined optical zones and one of the plurality of narrow and/or annular conjoined optical zones form a single cycle of oscillation of power (e.g., one or both of sagittal and tangential) around the base power profile (e.g., the base power profile of the central optical zone).

C22. The ophthalmic lens of any of the C examples, wherein a combination of at least one or more of the central optical zone size, the plurality of narrow and/or annular conjoined optical zones, the front surface curvature, lens thickness, back surface curvature, and the refractive index may be configured to form a power profile across the central and peripheral optical zones such that the ophthalmic lens forms on-axis focal points and off-axis focal points over a substantially wide range of vergences to provide an appropriate range of light energy distributions along the optical axis and across the retinal image plane that may correct/treat the refractive condition of the eye by extending the depth of focus along the optical axis at least in part on and/or in front of the retina of the eye to extend the depth of focus and/or to reduce the light intensity at a retinal image plane to reduce, mitigate or prevent one or more night vision disturbances that accompany the use of such ophthalmic devices.

C23. The ophthalmic lens of any of the C examples, wherein the power profile in the annular peripheral optical zone comprises a plurality of narrow and/or annular conjoined optical zones and wherein light rays from the plurality of narrow and/or annular conjoined optical zones provide a low light energy.

C24. The ophthalmic lens of any of the C examples, wherein an interference from light rays created by the plurality of narrow and/or annular conjoined optical zones increases and/or decreases from the anterior most image plane from retina to the posterior most (e.g., retinal) image plane or decreases from the retinal image plane (or another image plane) to at least one of the anterior most image plane and the posterior most image plane.

C25. The ophthalmic lens of any of the C examples, wherein any combination of at least one or more of the central optical zone diameter and/or the power profile of at least a portion of the ophthalmic lens may be used to provide a desirable condition to reduce/minimize light interference on in-focus images by out-of-focus images and/or to reduce, or mitigate, or prevent one or more night vision disturbances (e.g. by adjusting one or more of on-axis and/or off-axis focal point and image plane location, light energy, image quality, total enclosed energy distribution, and/or depth of focus).

C26. The ophthalmic lens of any of the C examples, wherein the ophthalmic lens provides, at least in part, an extended depth of focus within the useable vergence ranges encountered by a user of the ophthalmic lens.

C27. The ophthalmic lens of any of the C examples, wherein the one or more on-axis focal points has a low light energy along the optical axis of the ophthalmic lens.

C28. The ophthalmic lens of any of the C examples, wherein the ophthalmic lens is configured to provide a low light energy formed on the retina.

C29. The ophthalmic lens of any of the C examples, wherein light rays that form one or more off-axis focal points may be distributed across a substantially wide range of vergences along the optical axis and in front of, on and/or behind the retinal image plane of an eye in use.

C30. The ophthalmic lens of any of the C examples, wherein the ophthalmic lens has a uniform or relatively uniform light intensity distribution across the retinal spot diagram.

C31. The ophthalmic lens of any of the C examples, wherein a total enclosed energy that results at the retinal image plane may be determined from a retinal spot diagram, and at least more than about 50% (e.g., 45%, 50%, and/or 55%) of the total enclosed energy may be distributed beyond a 35 μm, 40 μm, 45 μm, 50 μm, 55 μm, 60 μm, 65 μm, 70 μm 75 μm, 80 μm, and/or 95 μm half chord diameter of the retinal spot diagram.

C32. The ophthalmic lens of any of the C examples, wherein a cumulative fraction of a total enclosed energy that results at the retinal image plane has an average slope of less than about 0.13 units/10 μm (e.g., about 0.11 units/10 μm, 0.12 units/10 μm, 0.125 units/10 μm, 0.13 units/10 μm, 0.14 units/10 μm, and/or 0.15 units/10 μm or less) over 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

C33. The ophthalmic lens of any of the C examples, wherein the central optical zone has a half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, about 0.1 mm or less.

C34. The ophthalmic lens of any of the C examples, wherein the ophthalmic lens may be one of a contact lens, an intraocular lens, and/or a spectacle lens.

### D Examples

D1. An ophthalmic lens comprising: an optical axis; and an optical zone comprising simultaneous vision and/or extended depth of focus optics; wherein the ophthalmic lens may be configured to provide low light energy levels within a usable vergence range of the ophthalmic lens.

D2. The ophthalmic lens of and of the D examples, wherein the ophthalmic lens has a uniform or relatively uniform light intensity distribution across the retinal spot diagram.

D3. The ophthalmic lens of any of the D examples, wherein a cumulative fraction of a total enclosed energy that results at the retinal image plane may be characterized by a retinal spot diagram, and at least more than about 50% (e.g., 45%, 50%, and/or 55%) of the total enclosed energy may be distributed beyond a 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

D4. The ophthalmic lens of any of the D examples, wherein a through focus retinal image quality (RIQ) of the ophthalmic lens comprises one or more independent peaks (e.g., 1, 2, 3, 4, 5, 6, 7, 8, 9, and/or 10 peaks), and wherein the maximum RIQ value of the one or more independent peaks may be less than about 0.45 (e.g., 0.42, 0.43, 0.44, 0.45, 0.46, 0.47 or 0.48).

D5. The ophthalmic lens of any of the D examples, wherein a through focus retinal image quality (RIQ) of the ophthalmic lens comprises one or more independent peaks (e.g., 1, 2, 3, 4, 5, 6, 7, 8, 9, and/or 10 peaks) over a vergence range of about ±3D (e.g., ±2.75 D, ±2.8 D, ±2.9 D, ±3D, ±3.1 D, ±3.2 D, and/or ±3.25 D), and wherein the maximum RIQ value of the one or more independent peaks may be less than about 0.45 (e.g., 0.42, 0.43, 0.44, 0.45, 0.46, 0.47 or 0.48).

D6. The ophthalmic lens of any of the D examples, wherein a through focus retinal image quality (RIQ) of the ophthalmic lens comprises one or more independent peaks (e.g., 1, 2, 3, 4, 5, 6, 7, 8, 9, and/or 10 peaks), and wherein the maximum RIQ value of the one or more independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48).

D7. The ophthalmic lens of any of the D examples, wherein a through focus retinal image quality (RIQ) of the ophthalmic lens comprises one or more independent peaks (e.g., 1, 2, 3, 4, 5, 6, 7, 8, 9, and/or 10 peaks) over a vergence range of about ±3D (e.g., ±2.75 D, ±2.8 D, ±2.9 D, ±3D, ±3.1 D, ±3.2 D, and/or ±3.25 D), and wherein the maximum RIQ value of the one or more independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48).

D8. The ophthalmic lens of any of the D examples, wherein the RIQ Area of the one or more independent peaks may be about 0.16 Units*Diopters (e.g., 0.13, 0.14, 0.15, 0.16, 0.17, 0.18 or 0.19) or less.

D9. The ophthalmic lens of any of the D examples, wherein the optics in the optical zone comprise a plurality of narrow and/or annular concentric optical zones and the power range between the absolute powers “p” and “m” components in the single power profile cycle (e.g., the peak to valley or P-to-V value) may be at least one of constant or varying (e.g., increasing, decreasing, and or randomly changing) in at least one direction across the optical zone.

D10. The ophthalmic lens of any of the D examples, wherein the ophthalmic lens (e.g., at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens.

D11. The ophthalmic lens of any of the D examples, wherein the ophthalmic lens (e.g., at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein a peak-to-valley (P-to-V) power range between the absolute powers of the “m” and “p” components of the cycle of the cyclical on-axis power profile in the sagittal direction may be about 200 D, about 150 D, about 100 D, about 75 D, about 50 D, about 40 D, about 30 D, about 20 D, about 10 D, about 5 D or less, about 4 D or less, about 3D or less, and/or about 2D or less.

D12. The ophthalmic lens of any of the D examples, wherein the ophthalmic lens (e.g., at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the peak-to-valley (P-to-V) power range between the absolute powers of the “m” and “p” components of the cycle of the cyclical power profile in the tangential direction may be about 600 D, about 500 D, about 400 D, about 300 D, about 200 D, about 175 D, about 150 D, about 125 D, about 100 D, about 75 D, about 60 D, about 50 D, about 40 D, about 35 D, and/or about 30 D or less.

D13. The ophthalmic lens of any of the D examples, wherein the ophthalmic lens (e.g., at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the frequency of the cyclical power profile may be about 0.5, 1, 1.5, 2, 3, 4, 5, 6, 7, 8, 9, 10, 15, 20, 50, and/or 100 cycles/mm.

D14. The ophthalmic lens of any of the D examples, wherein the optical zone comprises a central optical zone, a peripheral optical zone, and at least one feature forming part of the optics of the optical zone located in at least one of the central optical zone and the peripheral optical zone, and selected to modify the base power profile and to form one or more off-axis focal points in front of, on, and/or behind a retinal image plane and reduce a focal point energy level at one or more image planes.

D15. The ophthalmic lens of any of the D examples, wherein the optics in the optical zone may be configured to form one or more off-axis focal points in front of, on, and/or behind a retinal image plane.

D16. The ophthalmic lens of any of the D examples, wherein the optics in the optical zone comprise at least one narrow optical zone incorporating one or more cyclical power profiles and forming one or more off-axis focal points and one or more on-axis focal points along the optical axis.

D17. The ophthalmic lens of any of the D examples, wherein the optics in the optical zone comprise a line curvature (e.g., a cyclical power profile formed by a line curvature).

D18. The ophthalmic lens of any of the D examples, wherein the optics in the optical zone comprise a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones.

D19. The ophthalmic lens of any of the D examples, wherein the optics in the optical zone comprise a plurality e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones that may be between about 20-2000 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, about 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 775 μm, 800 μm, 825 μm, 850 μm, 875 μm, 900 μm, 925 μm, 950 μm, 975 μm, 1000 μm, 1025 μm, 1050 μm, 1075 μm, 1100 μm, 1125 μm, 1150 μm, 1175 μm, 1200 μm, 1225 μm, 1250 μm, 1275 μm, 1300 μm, 1325 μm, 1350 μm, 1375 μm, 1400 μm, 1525 μm, 1550 μm, 1575 μm, 1600 μm, 1625 μm, 1650 μm, 1675 μm, 1700 μm, 1725 μm, 1750 μm, 1775 μm, 1800 μm, 1825 μm, 1850 μm, 1875 μm, 1900 μm, 1925 μm, 1950 μm, 1975 μm, 2000 μm, 2025 μm, 2050 μm, 2075 μm, and/or 2100 μm wide).

D20. The ophthalmic lens of any of the D examples, wherein the optics in the optical zone comprise a plurality of narrow and/or annular concentric optical zones located on at least one of a front surface and/or a back surface of the ophthalmic lens and formed by line curvatures.

D21. The ophthalmic lens of any of the D examples, wherein the optics in the optical zone comprise a plurality of narrow and/or annular concentric optical zones and a net resultant power profile of the narrow and/or annular zones of the peripheral zone may be at least one of relatively more positive in power than the central zone, relatively more negative in power than the central zone, and/or about the same power as the central zone.

D22. The ophthalmic lens of any of the D examples, wherein the optics in the optical zone comprise a plurality of narrow and/or annular concentric optical zones and the plurality of narrow and/or annular concentric zones may be conjoined (e.g., the spacing between the two adjacent optical zones may be substantially zero and the innermost and the outermost portion of the surface curvature of the narrow and/or annular concentric zones transition to the base curve) with an adjacent narrow and/or annular concentric optical zone.

D23. The ophthalmic lens of any of the D examples, wherein the optics in the optical zone comprise a plurality of narrow and/or annular concentric optical zones and the plurality of narrow and/or annular concentric zones may be spaced apart from one another so as to create an alternating pattern where the spacing between the two adjacent optical zones may be non-zero.

D24. The ophthalmic lens of any of the D examples, wherein the optics in the optical zone comprise a plurality of narrow and/or annular concentric optical zones and the plurality of narrow and/or annular concentric zones may be configured so that the innermost and outermost portions of at least one of the narrow and/or annular concentric optical zones may be geometrically normal to the surface and provides a lateral separation of the focal points (e.g., infinite number of focal points) formed by the annular narrow optical zones from the optical axis.

D25. The ophthalmic lens of any of the D examples, wherein the optics in the optical zone comprise a plurality of narrow and/or annular concentric optical zones and the light energy and/or image quality formed by the plurality of narrow and/or annular concentric optical zones may be substantially similar and/or dissimilar.

D26. The ophthalmic lens of any of the D examples, wherein the optics in the optical zone comprise a plurality of narrow and/or annular concentric optical zones and one of the plurality of narrow and/or annular concentric optical zones form a single cycle of oscillation of power (e.g., one or both of sagittal and tangential) around the base power profile (e.g., the base power profile of the central optical zone).

D27. The ophthalmic lens of any of the D examples, wherein a combination of at least one or more of the central optical zone size, the plurality of narrow and/or annular concentric optical zones, the front surface curvature, lens thickness, back surface curvature, and the refractive index may be configured to form a power profile across the central and peripheral optical zones such that the ophthalmic lens forms on-axis focal points and off-axis focal points over a substantially wide range of vergences to provide an appropriate range of light energy distributions along the optical axis and across the retinal image plane that correct/treat the refractive condition of the eye by extending the depth of focus along the optical axis at least in part on and/or in front of the retina of the eye to extend the depth of focus and reduce the light intensity at a retinal plane during use to reduce, mitigate or prevent one or more night vision disturbances that accompany the use of such ophthalmic devices.

D28. The ophthalmic lens of any of the D examples, wherein the optics in the optical zone comprise a plurality of narrow and/or annular concentric optical zones and wherein light rays from the plurality of narrow and/or annular concentric optical zones has a lower light intensity.

D29. The ophthalmic lens of any of the D examples, wherein an interference from light rays created by the plurality of narrow and/or annular concentric optical zones increases and/or decreases from the anterior most image plane from retina to the posterior most (e.g., retinal) image plane or decreases from the retinal image plane (or another image plane) to at least one of the anterior most image plane and the posterior most image plane.

D30. The ophthalmic lens of any of the D examples, wherein any combination of at least one or more of a central optical zone diameter and/or a power profile of at least a portion of the ophthalmic lens may be used to provide a desirable condition to reduce or reduce/minimize light interference on in-focus images by out-of-focus images and/or to reduce, mitigate, or prevent one or more night vision disturbances (e.g. by adjusting one or more of on-axis and/or off-axis focal point and image plane location, light energy, image quality, total enclosed energy distribution, and/or depth of focus).

D31. The ophthalmic lens of any of the D examples, wherein, any combination of one or more of the number of narrow and/or annular concentric optical zones and/or width and/or sagittal power profile and/or tangential power profile and/or boundary power profile and/or m:p ratio (e.g., RMS) and/or P-to-V value and/or surface curvature and/or lateral separation and/or spacing and/or surface location of the optical zones may be used to provide a desirable condition to extend depth of focus, to reduce focal point energy levels, to reduce/minimize light interference on in-focus images by out-of-focus images and/or to reduce, or mitigate, or prevent one or more night vision disturbances (e.g., by adjusting one or more of on-axis and/or off-axis focal point and image plane location, light energy, image quality, total enclosed energy distribution, and/or depth of focus).

D32. The ophthalmic lens of any of the D examples, wherein light rays that form one or more off-axis focal points may be distributed across a substantially wide range of vergences along the optical axis and in front of, on and/or behind the retinal image plane of an eye in use.

D33. The ophthalmic lens of any of the D examples, wherein a central optical zone has a half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, about 0.1 mm or less.

D34. The ophthalmic lens of any of the D examples, wherein the optics in the optical zone may be configured to reduce, mitigate or prevent one or more night vision disturbances (e.g., any combination of one or more of glare, haloes and/or starbursts).

D35. The ophthalmic lens of any of the D examples, wherein the ophthalmic lens may be one of a contact lens, an intraocular lens, and/or a spectacle lens.

### E Examples

E1. An ophthalmic lens comprising: an optical axis; an optical zone comprising simultaneous vision and/or extended depth of focus optics; wherein a cumulative fraction of a total enclosed energy that results at the retinal image plane may be characterized by a retinal spot diagram, and at least more than about 50% (e.g., 45%, 50%, and/or 55%) of the total enclosed energy may be distributed beyond a 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

E2. The ophthalmic lens of and of the E examples, wherein the ophthalmic lens has a uniform or relatively uniform light intensity distribution across the retinal spot diagram.

E3. The ophthalmic lens of any of the E examples, wherein a through focus retinal image quality (RIQ) of the ophthalmic lens comprises one or more independent peaks (e.g., 1, 2, 3, 4, 5, 6, 7, 8, 9, and/or 10 peaks), and wherein the maximum RIQ value of the one or more independent peaks may be less than about 0.45 (e.g., 0.42, 0.43, 0.44, 0.45, 0.46, 0.47 or 0.48).

E4. The ophthalmic lens of any of the E examples, wherein a through focus retinal image quality (RIQ) of the ophthalmic lens comprises one or more independent peaks (e.g., 1, 2, 3, 4, 5, 6, 7, 8, 9, and/or 10 peaks) over a vergence range of about ±3D (e.g., ±2.75 D, ±2.8 D, ±2.9 D, ±3D, ±3.1 D, ±3.2 D, and/or ±3.25 D), and wherein the maximum RIQ value of the one or more independent peaks may be less than about 0.45 (e.g., 0.42, 0.43, 0.44, 0.45, 0.46, 0.47 or 0.48).

E5. The ophthalmic lens of any of the E examples, wherein a through focus retinal image quality (RIQ) of the ophthalmic lens comprises one or more independent peaks (e.g., 1, 2, 3, 4, 5, 6, 7, 8, 9, and/or 10 peaks), and wherein the maximum RIQ value of the one or more independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48).

E6. The ophthalmic lens of any of the E examples, wherein a through focus retinal image quality (RIQ) of the ophthalmic lens comprises one or more independent peaks (e.g., 1, 2, 3, 4, 5, 6, 7, 8, 9, and/or 10 peaks) over a vergence range of about ±3D (e.g., ±2.75 D, ±2.8 D, ±2.9 D, ±3D, ±3.1 D, ±3.2 D, and/or ±3.25 D), and wherein the maximum RIQ value of the one or more independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48).

E7. The ophthalmic lens of any of the E examples, wherein the RIQ Area of the one or more independent peaks may be about 0.16 Units*Diopters (e.g., 0.13, 0.14, 0.15, 0.16, 0.17, 0.18 or 0.19) or less.

E8. The ophthalmic lens of any of the E examples, wherein the optics in the optical zone comprise a plurality of narrow and/or annular concentric optical zones and the power range between the absolute powers of “p” and “m” components in the single power profile cycle (e.g., the peak to valley or P-to-V value) may be at least one of constant or varying (e.g., increasing, decreasing, and or randomly changing) in at least one direction across the optical zone.

E9. The ophthalmic lens of any of the E examples, wherein, any combination of one or more of the number of narrow and/or annular concentric optical zones and/or width and/or sagittal power profile and/or tangential power profile and/or boundary power profile and/or m:p ratio (e.g., RMS) and/or P-to-V value and/or surface curvature and/or lateral separation and/or spacing and/or surface location of the optical zones may be used to provide a desirable condition to extend depth of focus, to reduce focal point energy levels, to reduce/minimize light interference on in-focus images by out-of-focus images and/or to reduce, mitigate, or prevent one or more night vision disturbances (e.g., by adjusting one or more of on-axis and/or off-axis focal point and image plane location, light energy, image quality, total enclosed energy distribution, and/or depth of focus).

E10. The ophthalmic lens of any of the E examples, wherein the ophthalmic lens (e.g., the at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens.

E11. The ophthalmic lens of any of the E examples, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein a peak-to-valley (P-to-V) power range between the absolute powers of the “m” and “p” components of the cycle of the cyclical power profile in the sagittal direction may be about 200 D, about 150 D, about 100 D, about 75 D, about 50 D, about 40 D, about 30 D, about 20 D, about 10 D, about 5 D or less, about 4 D or less, about 3D or less, and/or about 2D or less.

E12. The ophthalmic lens of any of the E examples, wherein the ophthalmic lens (e.g., the at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the peak-to-valley (P-to-V) power range between the absolute powers of the “m” and “p” components of the cycle of the cyclical off-axis power profile in the tangential direction may be about 600 D, about 500 D, about 400 D, about 300 D, about 200 D, about 175 D, about 150 D, about 125 D, about 100 D, about 75 D, about 60 D, about 50 D, about 40 D, about 35 D, and/or about 30 D or less.

E13. The ophthalmic lens of any of the E examples, wherein the ophthalmic lens (e.g., the at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the frequency of the cyclical power profile may be about 0.5, 1, 1.5, 2, 3, 4, 5, 6, 7, 8, 9, 10, 15, 20, 50, and/or 100 cycles/mm.

E14. The ophthalmic lens of any of the E examples, wherein the optical zone comprises a central optical zone, a peripheral optical zone, and at least one feature forming part of the optics of the optical zone located in at least one of the central optical zone and the peripheral optical zone, and selected to modify the base power profile and to form one or more off-axis focal points in front of, on, and/or behind a retinal image plane and reduce a focal point energy level at one or more image planes.

E15. The ophthalmic lens of any of the E examples, wherein the optics in the optical zone may be configured to form one or more off-axis focal points in front of, on, and/or behind a retinal image plane.

E16. The ophthalmic lens of any of the E examples, wherein the optics in the optical zone comprise at least one narrow optical zone incorporating one or more cyclical power profiles and forming one or more off-axis focal points and one or more on-axis focal points along the optical axis.

E17. The ophthalmic lens of any of the E examples, wherein the optics in the optical zone comprise a line curvature (e.g., a cyclical power profile formed by a line curvature).

E18. The ophthalmic lens of any of the E examples, wherein the optics in the optical zone comprise a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones.

E19. The ophthalmic lens of any of the E examples, wherein the optics in the optical zone comprise a plurality e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones that may be between about 20-2000 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, about 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 775 μm, 800 μm, 825 μm, 850 μm, 875 μm, 900 μm, 925 μm, 950 μm, 975 μm, 1000 μm, 1025 μm, 1050 μm, 1075 μm, 1100 μm, 1125 μm, 1150 μm, 1175 μm, 1200 μm, 1225 μm, 1250 μm, 1275 μm, 1300 μm, 1325 μm, 1350 μm, 1375 μm, 1400 μm, 1525 μm, 1550 μm, 1575 μm, 1600 μm, 1625 μm, 1650 μm, 1675 μm, 1700 μm, 1725 μm, 1750 μm, 1775 μm, 1800 μm, 1825 μm, 1850 μm, 1875 μm, 1900 μm, 1925 μm, 1950 μm, 1975 μm, 2000 μm, 2025 μm, 2050 μm, 2075 μm, and/or 2100 μm wide).

E20. The ophthalmic lens of any of the E examples, wherein the optics in the optical zone comprise a plurality of narrow and/or annular concentric optical zones located on at least one of a front surface and/or a back surface of the ophthalmic lens and formed by line curvatures.

E21. The ophthalmic lens of any of the E examples, wherein the optics in the optical zone comprise a plurality of narrow and/or annular concentric optical zones and a net resultant power profile of the narrow and/or annular zones of the peripheral zone may be at least one of relatively more positive in power than the central zone, relatively more negative in power than the central zone, and/or about the same power as the central zone.

E22. The ophthalmic lens of any of the E examples, wherein the optics in the optical zone comprise a plurality of narrow and/or annular concentric optical zones and the plurality of narrow and/or annular concentric zones may be conjoined (e.g., the spacing between the two adjacent optical zones may be substantially zero and the innermost and the outermost portion of the surface curvature of the narrow and/or annular concentric zones transition to the base curve) with an adjacent narrow optical zone.

E23. The ophthalmic lens of any of the E examples, wherein the optics in the optical zone comprise a plurality of narrow and/or annular concentric optical zones and the plurality of narrow and/or annular concentric zones may be spaced apart from one another so as to create an alternating pattern where the spacing between the two adjacent optical zones may be non-zero.

E24. The ophthalmic lens of any of the E examples, wherein the optics in the optical zone comprise a plurality of narrow and/or annular concentric optical zones and the plurality of narrow and/or annular concentric zones may be configured so that the innermost and outermost portions of at least one of the narrow and/or annular concentric optical zones may be geometrically normal to the surface and provides a lateral separation of the focal points (e.g., infinite number of focal points) formed by the narrow and/or annular concentric optical zones from the optical axis.

E25. The ophthalmic lens of any of the E examples, wherein the optics in the optical zone comprise a plurality of narrow and/or annular concentric optical zones and the light energy and/or image quality formed by the plurality of narrow and/or annular concentric optical zones may be substantially similar and/or dissimilar.

E26. The ophthalmic lens of any of the E examples, wherein the optics in the optical zone comprise a plurality of narrow and/or annular concentric optical zones and one of the plurality of narrow and/or annular concentric optical zones form a single cycle of oscillation of power (e.g., one or both of sagittal and tangential) around the base power profile (e.g., the base power profile of the central optical zone).

E27. The ophthalmic lens of any of the E examples, wherein a combination of at least one or more of the central optical zone size, the plurality of narrow and/or annular concentric optical zones, the front surface curvature, lens thickness, back surface curvature, and the refractive index may be configured to form a power profile across the central and peripheral optical zones such that the ophthalmic lens forms on-axis focal points and off-axis focal points over a substantially wide range of vergences to provide an appropriate range of light energy distributions along the optical axis and across the retinal image plane that correct/treat the refractive condition of the eye by extending the depth of focus along the optical axis at least in part on and/or in front of the retina of the eye to extend the depth of focus and/or to and reduce the light intensity at a retinal plane during use to reduce, mitigate or prevent one or more night vision disturbances that accompany the use of such ophthalmic devices.

E28. The ophthalmic lens of any of the E examples, wherein the optics in the optical zone comprise a plurality of narrow and/or annular concentric optical zones and wherein light rays from the plurality of narrow and/or annular concentric optical zones has a lower light intensity.

E29. The ophthalmic lens of any of the E examples, wherein an interference from light rays created by the plurality of narrow and/or annular concentric optical zones increases and/or decreases from the anterior most image plane from retina to the posterior most (e.g., retinal) image plane or decreases from the retinal image plane (or another image plane) to at least one of the anterior most image plane and the posterior most image plane.

E30. The ophthalmic lens of any of the E examples, wherein and combination of at least one or more of a central optical zone diameter and/or a power profile of at least a portion of the ophthalmic lens may be used to provide a desirable condition to reduce or reduce/minimize light interference on in-focus images by out-of-focus images and/or to reduce, or mitigate, or prevent one or more night vision disturbances (e.g., by adjusting one or more of on-axis and/or off-axis focal point and image plane location, light energy, image quality, total enclosed energy distribution, and/or depth of focus).

E31. The ophthalmic lens of any of the E examples, wherein light rays that form one or more off-axis focal points may be distributed across a substantially wide range of vergences along the optical axis and in front of, on and/or behind the retinal image plane of an eye in use.

E32. The ophthalmic lens of any of the E examples, wherein a central optical zone has a half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, about 0.1 mm or less.

E33. The ophthalmic lens of any of the E examples, wherein the optics in the optical zone may be configured to reduce, mitigate or prevent one or more night vision disturbances (e.g., any combination of one or more of glare, haloes and/or starbursts).

E34. The ophthalmic lens of any of the E examples, wherein the ophthalmic lens may be one of a contact lens, an intraocular lens, and/or a spectacle lens.

### F Examples

F1. An ophthalmic lens comprising: an optical axis; an optical zone comprising simultaneous vision and/or extended depth of focus optics; wherein a through focus retinal image quality (RIQ) of the ophthalmic lens comprises one or more independent peaks (e.g., 1, 2, 3, 4, 5, 6, 7, 8, 9, and/or 10 peaks), and wherein the maximum RIQ value of the one or more independent peaks may be less than about 0.45 (e.g., 0.42, 0.43, 0.44, 0.45, 0.46, 0.47 or 0.48).

F2. The ophthalmic lens of any of the F examples, wherein a through focus retinal image quality (RIQ) of the ophthalmic lens comprises one or more independent peaks (e.g., 1, 2, 3, 4, 5, 6, 7, 8, 9, and/or 10 peaks) over a vergence range of about ±3D (e.g., ±2.75 D, ±2.8 D, ±2.9 D, ±3D, ±3.1 D, ±3.2 D, and/or ±3.25 D), and wherein the maximum RIQ value of the one or more independent peaks may be less than about 0.45 (e.g., 0.42, 0.43, 0.44, 0.45, 0.46, 0.47 or 0.48).

F3. The ophthalmic lens of any of the F examples, wherein a through focus retinal image quality (RIQ) of the ophthalmic lens comprises one or more independent peaks (e.g., 1, 2, 3, 4, 5, 6, 7, 8, 9, and/or 10 peaks), and wherein the maximum RIQ value of the one or more independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48).

F4. The ophthalmic lens of any of the F examples, wherein a through focus retinal image quality (RIQ) of the ophthalmic lens comprises one or more independent peaks (e.g., 1, 2, 3, 4, 5, 6, 7, 8, 9, and/or 10 peaks) over a vergence range of about ±3D (e.g., ±2.75 D, ±2.8 D, ±2.9 D, ±3D, ±3.1 D, ±3.2 D, and/or ±3.25 D), and wherein the maximum RIQ value of the one or more independent peaks may be between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48).

F5. The ophthalmic lens of any of the F examples, wherein the optics in the optical zone comprise a plurality of narrow and/or annular concentric optical zones and between the power range between the absolute powers of “p” and “m” components in the single power profile cycle (e.g., the peak to valley or P-to-V value) may be at least one of constant or varying (e.g., increasing, decreasing, and or randomly changing) in at least one direction across the optical zone.

F6. The ophthalmic lens of any of the F examples, wherein, any combination of one or more of the number of narrow and/or annular concentric optical zones and/or width and/or sagittal power profile and/or tangential power profile and/or boundary power profile and/or m:p ratio (e.g., RMS) and/or P:V value and/or surface curvature and/or lateral separation and/or spacing and/or surface location of the optical zones may be used to provide a desirable condition to extend depth of focus, to reduce focal point energy levels, to reduce/minimize light interference on in-focus images by out-of-focus images and/or to reduce, or mitigate, or prevent one or more night vision disturbances (e.g., by adjusting one or more of on-axis and/or off-axis focal point and image plane location, light energy, image quality, total enclosed energy distribution, and/or depth of focus).

F7. The ophthalmic lens of any of the F examples, wherein the ophthalmic lens (e.g., at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens.

F8. The ophthalmic lens of any of the F examples, wherein the ophthalmic lens (e.g., at least one feature of the ophthalmic lens) comprises a cyclical power profile comprising one or more cycles across the central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein a peak-to-valley (P-to-V) power range between the absolute powers of the “m” and “p” components of the cycle of the cyclical on-axis power profile in the sagittal direction may be about 200 D, about 150 D, about 100 D, about 75 D, about 50 D, about 40 D, about 30 D, about 20 D, about 10 D, about 5 D or less, about 4 D or less, about 3D or less, and/or about 2D or less.

F9. The ophthalmic lens of any of the F examples, wherein the ophthalmic lens (e.g., the at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the peak-to-valley (P-to-V) power range between the absolute powers of the “m” and “p” components of the cycle of the cyclical off-axis power profile in the tangential direction about 600 D, about 500 D, about 400 D, about 300 D, about 200 D, about 175 D, about 150 D, about 125 D, about 100 D, about 75 D, about 60 D, about 50 D, about 40 D, about 35 D, and/or about 30 D or less.

F10. The ophthalmic lens of any of the F examples, wherein the ophthalmic lens (e.g., the at least one feature of the ophthalmic lens) comprises a cyclical power profile comprising one or more cycles across a central and/or peripheral optical zone of the ophthalmic lens and the cycle of the cyclical power profile incorporates a “m” component that may be relatively more negative in power than the base power profile of the ophthalmic lens and a “p” component that may be relatively more positive in power than the base power profile of the ophthalmic lens; and wherein the frequency of the cyclical power profile may be about 0.5, 1, 1.5, 2, 3, 4, 5, 6, 7, 8, 9, 10, 15, 20, 50, and/or 100 cycles/mm.

F11. The ophthalmic lens of any of the F examples, wherein the ophthalmic lens may be configured to provide low light energy levels within a usable vergence range of the ophthalmic lens.

F12. The ophthalmic lens of any of the F examples, wherein the ophthalmic lens has a uniform or relatively uniform light intensity distribution across the retinal spot diagram.

F13. The ophthalmic lens of any of the F examples, wherein a cumulative fraction of a total enclosed energy that results at the retinal image plane may be characterized by a retinal spot diagram, and at least more than about 50% (e.g., 45%, 50%, and/or 55%) of the total enclosed energy may be distributed beyond a 35 μm, 40 μm, 45 μm, 50 μm, 55 μm, 60 μm, 65 μm, 70 μm, 75 μm, 80 μm, and/or 95 μm half chord diameter of the retinal spot diagram and/or an interval slope over any 20 μm (e.g., 17 μm, 18 μm, 19 μm, 20 μm, 21 μm, 22 μm, 23 μm, or 24 μm) half chord interval across the spot diagram of not greater than about 0.13 units/10 μm (e.g., not greater than about 0.11 units/10 μm, 0.12 units/10 μm, 0.13 units/10 μm, 0.14 units/10 μm, and/or 0.15 units/10 μm).

F14. The ophthalmic lens of any of the F examples, wherein the RIQ Area of the one or more independent peaks may be about 0.16 Units*Diopters (e.g., 0.13, 0.14, 0.15, 0.16, 0.17, 0.18 or 0.19) or less.

F15. The ophthalmic lens of any of the F examples, wherein the optical zone comprises a central optical zone, a peripheral optical zone, and at least one feature forming part of the optics of the optical zone located in at least one of the central optical zone and the peripheral optical zone, and selected to modify the base power profile and to form one or more off-axis focal points in front of, on, and/or behind a retinal image plane and reduce a focal point energy level at one or more image planes.

F16. The ophthalmic lens of any of the F examples, wherein the optics in the optical zone may be configured to form one or more off-axis focal points in front of, on, and/or behind a retinal image plane.

F17. The ophthalmic lens of any of the F examples, wherein the optics in the optical zone comprise at least one narrow optical zone incorporating one or more cyclical power profiles and forming one or more off-axis focal points and one or more on-axis focal points along the optical axis.

F18. The ophthalmic lens of any of the F examples, wherein the optics in the optical zone comprise a line curvature (e.g., a cyclical power profile formed by a line curvature).

F19. The ophthalmic lens of any of the F examples, wherein the optics in the optical zone comprise a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones.

F20. The ophthalmic lens of any of the F examples, wherein the optics in the optical zone comprise a plurality e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones that may be between about 20-2000 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, about 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 775 μm, 800 μm, 825 μm, 850 μm, 875 μm, 900 μm, 925 μm, 950 μm, 975 μm, 1000 μm, 1025 μm, 1050 μm, 1075 μm, 1100 μm, 1125 μm, 1150 μm, 1175 μm, 1200 μm, 1225 μm, 1250 μm, 1275 μm, 1300 μm, 1325 μm, 1350 μm, 1375 μm, 1400 μm, 1525 μm, 1550 μm, 1575 μm, 1600 μm, 1625 μm, 1650 μm, 1675 μm, 1700 μm, 1725 μm, 1750 μm, 1775 μm, 1800 μm, 1825 μm, 1850 μm, 1875 μm, 1900 μm, 1925 μm, 1950 μm, 1975 μm, 2000 μm, 2025 μm, 2050 μm, 2075 μm, and/or 2100 μm wide).

F21. The ophthalmic lens of any of the F examples, wherein the optics in the optical zone comprise a plurality of narrow and/or annular concentric optical zones located on at least one of a front surface and/or a back surface of the ophthalmic lens and formed by line curvatures.

F22. The ophthalmic lens of any of the F examples, wherein the optics in the optical zone comprise a plurality of narrow and/or annular concentric optical zones and a net resultant power profile of the narrow and/or annular zones of the peripheral zone may be at least one of relatively more positive in power than the central zone, relatively more negative in power than the central zone, and/or about the same power as the central zone.

F23. The ophthalmic lens of any of the F examples, wherein the optics in the optical zone comprise a plurality of narrow and/or annular concentric optical zones and the plurality of narrow and/or annular concentric zones may be conjoined (e.g., the spacing between the two adjacent optical zones may be substantially zero and the innermost and the outermost portion of the surface curvature of the narrow and/or annular concentric zones transition to the base curve) with an adjacent narrow and/or annular concentric optical zone.

F24. The ophthalmic lens of any of the F examples, wherein the optics in the optical zone comprise a plurality of narrow and/or annular concentric optical zones and the plurality of narrow and/or annular concentric zones may be spaced apart from one another so as to create an alternating pattern where the spacing between the two adjacent optical zones may be non-zero.

F25. The ophthalmic lens of any of the F examples, wherein the optics in the optical zone comprise a plurality of narrow and/or annular concentric optical zones and the plurality of narrow and/or annular concentric zones may be configured so that the innermost and outermost portions of at least one of the narrow and/or annular concentric optical zones may be geometrically normal to the surface and provides a lateral separation of the focal points (e.g., infinite number of focal points) formed by the narrow and/or annular optical zones from the optical axis.

F26. The ophthalmic lens of any of the F examples, wherein the optics in the optical zone comprise a plurality of narrow and/or annular concentric optical zones and the light energy and/or image quality formed by the plurality of narrow and/or annular concentric optical zones may be substantially similar and/or dissimilar.

F27. The ophthalmic lens of any of the F examples, wherein the optics in the optical zone comprise a plurality of narrow and/or annular concentric optical zones and one of the plurality of narrow and/or annular concentric optical zones form a single cycle of oscillation of power (e.g., one or both of sagittal and tangential) around the base power profile (e.g., the base power profile of the central optical zone).

F28. The ophthalmic lens of any of the F examples, wherein a combination of at least one or more of the central optical zone size, the plurality of narrow and/or annular concentric optical zones, the front surface curvature, lens thickness, back surface curvature, and the refractive index may be configured to form a power profile across the central and peripheral optical zones such that the ophthalmic lens forms on-axis focal points and off-axis focal points over a substantially wide range of vergences to provide an appropriate range of light energy distributions along the optical axis and across the retinal image plane that may correct/treat the refractive condition of the eye by extending the depth of focus along the optical axis at least in part on and/or in front of the retina of the eye to extend the depth of focus and/or to reduce the light intensity at a retinal plane during use to reduce, mitigate or prevent one or more night vision disturbances that accompany the use of such ophthalmic devices.

F29. The ophthalmic lens of any of the F examples, wherein the optics in the optical zone comprise a plurality of narrow and/or annular concentric optical zones and wherein light rays from the plurality of narrow and/or annular concentric optical zones has a lower light intensity.

F30. The ophthalmic lens of any of the F examples, wherein an interference from light rays created by the plurality of narrow and/or annular concentric optical zones zones increases and/or decreases from the anterior most image plane from retina to the posterior most (e.g., retinal) image plane or decreases from the retinal image plane (or another image plane) to at least one of the anterior most image plane and the posterior most image plane.

F31. The ophthalmic lens of any of the F examples, wherein and combination of at least one or more of a central optical zone diameter and/or a power profile of at least a portion of the ophthalmic lens may be used to provide a desirable condition to reduce or reduce/minimize light interference on in-focus images by out-of-focus images and/or to reduce, mitigate, or prevent one or more night vision disturbances (e.g. by adjusting one or more of on-axis and/or off-axis focal point and image plane location, light energy, image quality, total enclosed energy distribution, and/or depth of focus).

F32. The ophthalmic lens of any of the F examples, wherein light rays that form one or more off-axis focal points may be distributed across a substantially wide range of vergences along the optical axis and in front of, on and/or behind the retinal image plane of an eye in use.

F33. The ophthalmic lens of any of the F examples, wherein a central optical zone has a half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, about 0.1 mm or less.

F34. The ophthalmic lens of any of the F examples, wherein the optics in the optical zone may be configured to reduce, mitigate or prevent one or more night vision disturbances (e.g., any combination of one or more of glare, haloes and/or starbursts).

F35. The ophthalmic lens of any of the F examples, wherein the ophthalmic lens may be one of a contact lens, an intraocular lens, and/or a spectacle lens.

### G Examples

G1. A method for managing an ocular condition comprising: utilizing an ophthalmic lens of any of the A, B, C, D, E, and F examples wherein the ophthalmic lens may be configured to provide low light energy levels within a usable vergence range of the ophthalmic lens.

### H Examples

H1. A system for managing an ocular condition comprising: any combination of one or more of the ophthalmic lens of any of the A, B, C, D, E, and F examples wherein the one or more ophthalmic lens may be configured to provide low light energy levels within a usable vergence range of the ophthalmic lens.

It will be understood that the embodiments disclosed and defined in this specification extends to all alternative combinations of two or more of the individual features mentioned or evident from the text or drawings. All of these different combinations constitute various alternative aspects of the present disclosure.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. An ophthalmic lens configured to correct and/or treat at least one condition of the eye (e.g., presbyopia, myopia, hyperopia, astigmatism, binocular vision disorders and/or visual fatigue syndrome) comprising:
a central optical zone;
a peripheral optical zone;
a base power profile; and
at least one feature selected to modify the base power profile and to form one or more off-axis focal points in front of, on, and/or behind a retinal image plane and reduce a focal point energy level at one or more image planes;
wherein the at least one feature is located on a front surface and/or a back surface of at least one of the central optical zone and the peripheral optical zone.

2. The ophthalmic lens of claim 1, wherein the at least one feature comprises at least one narrow optical zone incorporating one or more cyclical power profiles and forming one or more off-axis focal points and one or more on-axis focal points along the optical axis.

3. The ophthalmic lens of claim 1, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D, ±3D, ±3. ID, ±3.2 D, and/or ±3.25 D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).

4. The ophthalmic lens of claim 1, wherein the ophthalmic lens provides a through focus retinal image quality (RIQ) with one or more (e.g., 1, 2, 3, 4, or 5) independent peaks (e.g., over a vergence range of about ±3D (e.g., ±2.75 D, ±2.8 D, ±2.9 D±3D, ±3. ID±3.2 D, and/or ±3.25 D)), and wherein an RIQ area of the one or more independent peaks is about 0.16 Units*Diopters (e.g., 0.13, 0.14, 0.15, 0.16, 0.17, 0.18 or 0.19) or less.

5. The ophthalmic lens of claim 1, wherein the ophthalmic lens provides a through focus retinal image quality (RIQ) with one or more (e.g., 1, 2, 3, 4, or 5) independent peaks (e.g., over a vergence range of about ±3.0 D (e.g., ±2.75 D, ±2.8 D, ±2.9 D, ±3D, ±3. ID, ±3.2 D, and/or ±3.25 D)), and wherein there is at least one or more independent peaks (e.g., 1, 2, 3, 4, or 5 peaks).

6. The ophthalmic lens of claim 1, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that is relatively more negative in power than the base power profile of the ophthalmic lens and a “p” component that is relatively more positive in power than the base power profile of the ophthalmic lens.

7. The ophthalmic lens of claim 1, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that is relatively more negative in power than the base power profile of the ophthalmic lens and a “p” component that is relatively more positive in power than the base power profile of the ophthalmic lens; and wherein a peak-to-valley (P-to-V) power range between the absolute powers of the “m” and “p” components of the cycle of the cyclical power profile in the sagittal direction is about 200 D, about 150 D, about 100 D, about 75 D, about 50 D, about 40 D, about 30 D, about 20 D, about 10 D, about 5 D or less, about 4 D less, about 3D or less, and/or about 2D or less.

8. The ophthalmic lens of claim 1, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that is relatively more negative in power than the base power profile of the ophthalmic lens and a “p” component that is relatively more positive in power than the base power profile of the ophthalmic lens; and wherein the peak-to-valley (P-to-V) power range between the absolute powers of the “m” and “p” components of the cycle of the cyclical power profile in the tangential direction is, about 600 D, about 500 D, about 400 D, about 300 D, about 200 D, about 175 D, about 150 D, about 125 D, about 100 D, about 75 D, about 60 D, about 50 D, about 40 D, about 35 D, and/or about 30 D or less.

9. The ophthalmic lens of claim 1, wherein the ophthalmic lens (e.g., the at least one feature of the ophthalmic lens) comprises a cyclical power profile comprising one or more cycles across the central and/or peripheral optical zone of the ophthalmic lens and the cycle of the cyclical power profile incorporates a “m” component that is relatively more negative in power than the base power profile of the ophthalmic lens and a “p” component that is relatively more positive in power than the base power profile of the ophthalmic lens; and wherein the frequency of the cyclical power profile is about 0.5, 1, 1.5, 2, 3, 4, 5, 6, 7, 8, 9, 10, 15, 20, 50 and/or 100 cycles/mm.

10. The ophthalmic lens of claim 1, wherein the at least one feature comprises a line curvature (e.g., a cyclical power profile formed by a line curvature).

11. The ophthalmic lens of claim 1, wherein the at least one feature comprises a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones.

12. The ophthalmic lens of claim 1, wherein the at least one feature comprises a plurality (e.g., 2, 3, 4, 5, 6, 7, 8, 9, 10, 11, 12, 13, 14, 15, 16, 17, 18, 19, 20, 21, 22, 23, 24, 25, or more) of narrow and/or annular concentric optical zones that are between about 20-2000 pm wide (e.g., about 15 pm, 20 pm, 30 pm, 40 pm, 50 pm, 60 pm, 70 pm, 75 pm, 80 pm, 90 pm, 100 pm, 110 pm, 120 pm, 125 pm, 130 pm, 140 pm, 150 pm, 160 pm, 170 pm, 175 pm, 180 pm, 190 pm, 200 pm, 210 pm, 220 pm, about 225 pm, 250 pm, 275 pm, 300 pm, 325 pm, 350 pm, 375 pm, 400 pm, 425 pm, 450 pm, 475 pm, 500 pm, 525 pm, 550 pm, 575 pm, 600 pm, 625 pm, 650 pm, 675 pm, 700 pm, 725 pm, 750 pm, 775 pm, 800 pm, 825 pm, 850 pm, 875 pm, 900 pm, 925 pm, 950 pm, 975 pm, 1000 pm, 1025 pm, 1050 pm, 1075 pm, 1100 pm, 1125 pm, 1150 pm, 1175 pm, 1200 pm, 1225 pm, 1250 pm, 1275 pm, 1300 pm, 1325 pm, 1350 pm, 1375 pm, 1400 pm, 1525 pm, 1550 pm, 1575 pm, 1600 pm, 1625 pm, 1650 pm, 1675 pm, 1700 pm, 1725 pm, 1750 pm, 1775 pm, 1800 pm, 1825 pm, 1850 pm, 1875 pm, 1900 pm, 1925 pm, 1950 pm, 1975 pm, 2000 pm, 2025 pm, 2050 pm, 2075 pm, and/or 2100 pm wide).

13. The ophthalmic lens of claim 1, wherein the at least one feature comprises a plurality of narrow and/or annular concentric optical zones located on at least one of the front surface and/or the back surface of the ophthalmic lens and formed by line curvatures.

14. The ophthalmic lens of claim 1, wherein the at least one feature comprises a plurality of narrow and/or annular concentric optical zones and a net resultant power profile of the narrow and/or annular concentric optical zones of the peripheral zone is at least one of relatively more positive in power than the central zone, relatively more negative in power than the central zone, and/or about the same power as the central zone.

15. The ophthalmic lens of claim 1, wherein the at least one feature comprises a plurality of narrow and/or annular concentric optical zones and the plurality of narrow and/or annular concentric zones are conjoined (e.g., the spacing between the two adjacent optical zones is substantially zero and the innermost and the outermost portion of the surface curvature of the narrow and/or annular concentric zones transition to the base curve) with an adjacent narrow and/or annular concentric optical zone.

16. The ophthalmic lens of claim 1, wherein the at least one feature comprises a plurality of narrow and/or annular concentric optical zones and the plurality of narrow and/or annular concentric zones are spaced apart from one another so as to create an alternating pattern where the base power profile (or a power other than the base power) alternates with the narrow and/or annular concentric zones.

17. The ophthalmic lens of claim 1, wherein the at least one feature comprises a plurality of narrow and/or annular concentric optical zones and the plurality of narrow and/or annular concentric zones are configured so that the innermost and outermost portions of at least one of the narrow and/or annular concentric optical zones is geometrically normal to the surface and provides a lateral separation of the focal points (e.g., infinite number of focal points) formed by the annular narrow and/or annular concentric optical zones from the optical axis.

18. The ophthalmic lens of claim 1, wherein the at least one feature comprises a plurality of narrow and/or annular concentric optical zones and the light energy and/or image quality formed by the plurality of narrow and/or annular concentric optical zones is substantially similar and/or dissimilar.

19. The ophthalmic lens of claim 1, wherein the at least one feature comprises a plurality of narrow and/or annular concentric optical zones and one of the plurality of narrow and/or annular concentric optical zones form a single cycle of oscillation of power (e.g., one or both of sagittal and tangential) around the base power profile (e.g., the base power profile of the central optical zone).

20. The ophthalmic lens of claim 1, wherein the at least one feature comprises a plurality of narrow and/or annular concentric optical zones and the power range between the absolute powers of “p” and “m” components in the single power profile cycle (e.g., the peak to valley or P-to-V value) is at least one of constant or varying (e.g., increasing, decreasing, and or randomly changing) in at least one direction across the optical zone.

21. The ophthalmic lens of claim 1, wherein a combination of at least one or more of the central optical zone size, the plurality of narrow and/or annular concentric optical zones, the front surface curvature, lens thickness, back surface curvature, and the refractive index are configured to form a power profile across the central and peripheral optical zones such that the ophthalmic lens forms on-axis focal points and off-axis focal points over a substantially wide range of vergences to provide an appropriate range of light energy distributions along the optical axis and across the retinal image plane that correct/treat the refractive condition of the eye by extending the depth of focus along the optical axis at least in part on and/or in front of the retina and/or behind the retina of the eye to extend the depth of focus and/or to reduce, mitigate or prevent one or more night vision disturbances that accompany the use of such ophthalmic devices.

22. The ophthalmic lens of claim 1, wherein the at least one feature comprises a plurality of narrow and/or annular concentric optical zones and wherein light rays from the plurality of narrow and/or annular concentric optical zones provide a low light energy.

23. The ophthalmic lens of claim 1, wherein an interference from light rays created by the plurality of narrow and/or annular concentric optical zones increases and/or decreases from the anterior most image plane from retina to the posterior most (e.g., retinal) image plane or decreases from the retinal image plane (or another image plane) to at least one of the anterior most image plane and the posterior most image plane.

24. The ophthalmic lens of claim 1, wherein any combination of at least one or more of the central optical zone diameter and/or the power profile of at least a portion of the ophthalmic lens are used to provide a desirable condition to reduce/minimize light interference on in-focus images by out-of-focus images and/or to reduce, mitigate, or prevent one or more night vision disturbances (e.g. by adjusting one or more of on-axis and/or off-axis focal point and image plane location, light energy, image quality, total enclosed energy distribution, and/or depth of focus).

25. The ophthalmic lens of claim 1, wherein, any combination of one or more of the number of narrow and/or annular concentric optical zones and/or width and/or sagittal power profile and/or tangential power profile and/or boundary power profile and/or m:p ratio (e.g., RMS) and/or P-to-V value and/or surface curvature and/or lateral separation and/or spacing and/or surface location of the optical zones are used to provide a desirable condition to extend depth of focus, to reduce focal point energy levels, to reduce/minimize light interference on in-focus images by out-of-focus images and/or to reduce, mitigate, or prevent one or more night vision disturbances (e.g., by adjusting one or more of on-axis and/or off-axis focal point and image plane location, light energy, image quality, total enclosed energy distribution, and/or depth of focus).

26. The ophthalmic lens of claim 1, wherein the ophthalmic lens provides, at least in part, an extended depth of focus within the useable vergence ranges encountered by a user of the ophthalmic lens.

27. The ophthalmic lens of claim 1, wherein the one or more on-axis focal points has a low light energy along the optical axis of the ophthalmic lens.

28. The ophthalmic lens of claim 1, wherein the ophthalmic lens is configured to provide a low light energy formed on the retina.

29. The ophthalmic lens of claim 1, wherein light rays that form one or more off-axis focal points are distributed across a substantially wide range of vergences along the optical axis and in front of, on, and/or behind the retinal image plane of an eye in use.

30. The ophthalmic lens of claim 1, wherein the ophthalmic lens has a uniform or relatively uniform light ray intensity distribution across the retinal spot diagram.

31. The ophthalmic lens of claim 1, wherein a total enclosed energy that results at the retinal image plane is determined from a retinal spot diagram, and at least more than about 50% (e.g., 45%, 50%, and/or 55%) of the total enclosed energy is distributed beyond a 35 pm, 40 pm, 45 pm, 50 pm, 55 pm, 60 pm, 65 pm, 70 pm, 75 pm, 80 pm, and/or 95 pm half chord diameter of the retinal spot diagram.

32. The ophthalmic lens of claim 1, wherein a cumulative fraction of a total enclosed energy that results at the retinal image plane has an average slope of less than about 0.13 units/10 pm (e.g., about 0.11 units/10 pm, 0.12 units/10 pm, 0.125 units/10 pm, 0.13 units/10 pm, 0.14 units/10 pm, and/or 0.15 units/10 pm or less) over 35 pm, 40 pm, 45 pm, 50 pm, 55 pm, 60 pm, 65 pm, 70 pm, 75 pm, 80 pm, and/or 95 pm half chord diameter of the retinal spot diagram and/or an interval slope over any 20 pm (e.g., 17 pm, 18 pm, 19 pm, 20 pm, 21 pm, 22 pm, 23 pm, or 24 pm) half chord interval across the spot diagram of not greater than about 0.13 units/10 pm (e.g., not greater than about 0.11 units/10 pm, 0.12 units/10 pm, 0.13 units/10 pm, 0.14 units/10 pm, and/or 0.15 units/10 pm).

33. The ophthalmic lens of claim 1, wherein the central optical zone has a half-chord diameter of about 5 mm, about 4 mm, about 3 mm, about 2 mm, about 1.75 mm, about 1.5 mm, about 1.25 mm, about 1.0 mm, about 0.5 mm, about 0.25 mm, about 0.1 mm or less.

34. The ophthalmic lens of claim 1, wherein the at least one feature is configured to reduce, mitigate and/or prevent one or more night vision disturbances (e.g., any combination of one or more of glare, haloes and/or starbursts).

35. The ophthalmic lens of claim 1, wherein the ophthalmic lens is one of a contact lens, an intraocular lens, and/or a spectacle lens.

36-70. (canceled)

71. An ophthalmic lens comprising:
a front surface;
a back surface;
a central optical zone;
an annular peripheral optical zone surrounding the central optical zone; and an optical design formed on at least one of the front surface or the back surface of the ophthalmic lens;
wherein the optical design comprises a power profile (e.g., a cyclical or non-cyclical power profile) in the central optical zone that forms at least one focal point along an optical axis (e.g., in front of, on and/or behind the retinal image plane); and
wherein the optical design comprises a power profile in the annular peripheral optical zone comprising at least one or more narrow and/or annular conjoined optical zones that have a cyclical power profile and form one or more off-axis focal points (e.g., in front of, on, and/or behind the retinal image plane)

72-139. (canceled)

140. An ophthalmic lens comprising:
an optical axis;
an optical zone comprising simultaneous vision and/or extended depth of focus optics; wherein a cumulative fraction of a total enclosed energy that results at the retinal image plane is characterized by a retinal spot diagram, and at least more than about 50% (e.g., 45%, 50%, and/or 55%) of the total enclosed energy is distributed beyond a 35 pm, 40 pm, 45 pm, 50 pm, 55 pm, 60 pm, 65 pm, 70 pm, 75 pm, 80 pm, and/or 95 pm half chord diameter of the retinal spot diagram and/or an interval slope over any 20 pm (e.g., 17 pm, 18 pm, 19 pm, 20 pm, 21 pm, 22 pm, 23 pm, or 24 pm) half chord interval across the spot diagram of not greater than about 0.13 units/10 pm (e.g., not greater than about 0.11 units/10 pm, 0.12 units/10 pm, 0.13 units/10 pm, 0.14 units/10 pm, and/or 0.15 units/10 pm).

141-208. (canceled)
